(19) United States
(12) Patent Application Publication
(10) Pub. No.: US 2026/0355257 A1
(43) Pub. Date: Nov. 12, 2026

(54) EQUINE UMBILICAL TISSUE AND METHODS OF USE

(71) Applicant: Equine Performance Labs, Austin, TX (US)

(72) Inventors: Haylee Ragland, Austin, TX (US); Alicia L. Bertone, Charlotte, NC (US); Heather Rogers, Austin, TX (US); Abraham William Silfen, Austin, TX (US)

(21) Appl. No.: 19/635,121

(22) Filed: Mar. 31, 2026

Related U.S. Application Data
(60) Provisional application No. 63/800,186, filed on May 5, 2025.
(60) Provisional application No. 63/884,785, filed on Sep. 19, 2025.

(51) Int. Cl. A61K 35/51; A01N 1/125; A61P 19/02; A61P 19/04

(52) U.S. Cl. CPC A61K 35/51; A01N 1/125; A61P 19/02; A61P 19/04

(57) ABSTRACT

Disclosed are compositions comprising fillet-derived equine umbilical tissue. Disclosed are methods of treating tissue defects and poor-quality healing such as arthritis/osis, desmitis/osis, and/or tendinitis/osis in a subject comprising administering one or more of the compositions disclosed herein to a subject in need thereof. Disclosed are methods of making compositions comprising fillet-derived equine umbilical tissue.

### CROSS-REFERENCE TO RELATED APPLICATIONS

This application claims the benefit of U.S. Provisional Patent Application No. 63/800,186, filed on May 5, 2025 and U.S. Provisional Patent Application No. 63/884,785, filed on Sep. 19, 2025, each of which is incorporated by reference herein in its entirety.

### BACKGROUND

The development of flowable particulate biologic scaffolds as grafts to supplement tissues in joints, tendons, and ligaments is in its early stages. These biologic scaffolds offer the advantages of being biodegradable and not a foreign substance, such as polyacrylamides or xenografts, providing the ability to select a tissue source that aligns with the structural support and biocompatibility of the intended tissue target. The most investigated tissue sources for microparticulate allografts in any species are birth tissues, including maternal placental tissue (chorioallantois), fetal tissue (amnionic membrane and fluid), and umbilical cord.

In placental mammals, the umbilical cord (UC) connects the developing fetus to the placenta and typically includes two umbilical arteries and one umbilical vein. These vessels are surrounded by an umbilical connective-tissue matrix and, in many cases, the cord is externally surrounded by the amnion. Portions of the UC can be used as a biological scaffold

### BRIEF SUMMARY

Disclosed are compositions comprising fillet-derived umbilical tissue (FDUT).

Disclosed are methods of using a composition comprising fillet-derived equine umbilical tissue.

Disclosed are methods of treating tissue defects comprising administering one or more of the compositions disclosed herein to a subject in need thereof.

Disclosed are methods of promoting healing at a surgical site of a subject comprising administering one or more of the disclosed compositions during or immediately following a surgical procedure to the open surgical site or closed surgical site.

Disclosed are methods of treating pain in a subject comprising administering one or more of the compositions disclosed herein to a subject in need thereof.

Disclosed are methods of treating inflammation in a subject comprising administering one or more of the compositions disclosed herein to a subject in need thereof.

Disclosed are methods comprising obtaining or having obtained an equine umbilical cord having a fillet portion and a branch portion; separating the fillet portion of the equine umbilical cord and the branch portion of the equine umbilical cord; removing the branch portion of the equine umbilical cord; and removing an outer membrane from the fillet portion of the equine umbilical cord; and removing the blood vessels; thereby producing a fillet-derived equine umbilical tissue.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-F shows selected synovial fluid cytokine concentrations (pg/mL) in normal horses injected into the carpal joint with equine fillet-derived umbilical tissue suspension or saline control expressed as a percentage of baseline. FIG. 1A shows an increase in Il-1ra after equine fillet-derived umbilical tissue suspension injection was significantly greater than Day 0 baseline and C (P<0.001) and remained greater than baseline values in 7 of 8 horses until the end of the study, a finding that did not occur in control. FIG. 1B shows Il-1 concentrations were low and did not significantly increase after equine fillet-derived umbilical tissue suspension injection. FIG. 1C shows the Il-1/Il-1ra ratio was an even greater difference from C due to the very low Il-1 that exaggerated the ratio change (P<0.001). FIG. 1D shows that there is a greater increase in VEGF with quine fillet-derived umbilical tissue suspension injection on days 1 and 3. FIG. 1E shows Il-10 is a potent anti-inflammatory cytokine with reported chondroprotective effects and was significantly greater (P<0.01) than both baseline and C after equine fillet-derived umbilical tissue suspension injection. FIG. 1F shows MCP-1 is a chemokine, a group of secreted proteins within the cytokine family, whose generic function is to induce cell migration. The significant (P<0.05) increase in MCP-1 after equine fillet-derived umbilical tissue suspension injection compared to baseline and C, along with rapid return to baseline values without any other increase in 20 days suggests lack of continued immune stimulation and inflammation in vivo.

FIG. 2 shows a diagram of the fillet and branch portions of the umbilical cord.

FIG. 3 shows a depiction of the removal of material (e.g., a hippomane) from the external surface of the equine umbilical cord.

FIG. 4 shows a depiction of the removal of the blood vessels from the umbilical tissue leaving the fillet-derived portion of umbilical tissue, Once the vessels are removed what's left is the Fillet-Derived Umbilical Tissue (FDUT).

FIGS. 5A and 5B shows a depiction showing an isolated perivascular layer (FIG. 5A) of the isolated intervascular and perivascular layers as a whole (FIG. 5B).

FIG. 6 shows a depiction of blood clots and red stretch marks, the red, bloody looking tissue that is cut/removed from the fillet portion of the umbilical cord.

FIGS. 7A and 7B shows a depiction of a cross section of the fillet portion of the umbilical cord (FIG. 7A) and also a cartoon illustration to show the anatomy of the equine umbilical cord (FIG. 7B).

FIG. 8 shows an image of a cross-section of the fillet portion used in the histology study, showing what can be described as three tubes connected by connective tissue.

FIGS. 9A and 9B show images of equicenta™CTM, an example of a filet-derived umbilical tissue. FIG. 9A shows the presence of filamentous matrix particles (large arrows) observed from the cytological evaluation of pelleted umbilical-derived tissue allograft (equicenta™CTM). Small arrows represent the potential of cellular components within the ECM. Bar=200 μm. FIG. 9B shows a panel of images demonstrates the aggregation of equicenta™CTM in culture medium with no digestion, after 1 hr of collagenase digestion, and after 30 minutes of trypsin digestion. Note the smaller particle size and few aggregates after enzymatic digestion.

FIG. 10 shows raw counts of CD9-captured particles per mL for each sample (e.g. ~1.0×10⁸ to 3.5×10⁸ particles/mL across samples 1-7).

FIG. 11 shows pie charts summarizing the fraction of CD9 only-labeled events relative to the total exosome capture (the CD9 slice is ~50-70% on average for samples 1-7). FIG. 11 also shows that when looking at co-expressed events containing CD9 the percentages are even higher.

FIG. 12 shows a heat map of the average colocalization counts for CD9, CD90, and CD 81. Key on the far right indicates the darker color at the bottom has lower counts while the lighter the color gets the higher counts.

FIG. 13 shows cumulative improvement in score (mean +/− SEM) for the clinical outcomes of lameness, pain and swelling recorded by study veterinarians over 90 days for 195 conditions for equicenta™ CTM or standard of care (control). FIG. 13 shows a significant improvement was noted by day 14 for pain and swelling and day 30 for lameness. (p<0.05) Follow-up was 98%-100% for all time points as reported in the table below the graph for each condition.

FIG. 14 shows cytokine and growth-factor release from FDUT microparticles over time.

FIG. 15 shows microscopy and protein concentration of equicenta™CTM in saline or after digestion with collagenase I or trypsin for 0, 0.5, 2, and/or 24 hours.

FIG. 16 shows TIMP and Col1A1 concentrations in a sample of equicenta™CTM in saline or digested with collagenase I or trypsin.

FIG. 17 shows isolation of extracellular vesicles (EVs) from different donors of equicenta™CTM and analysis for marker expression.

FIG. 18 shows an experimental design. (1) Cartilage and synovium explants were collected from the femoropatellar joint of three horses and placed in 6-well culture dishes, generating 36 experimental wells for each explant tissue studied. (2) After a period of equilibration, explants from each horse were pre-exposed to PBS (negative control) or equicenta™CTM in media for 48 hours in triplicate. (3) After washing, PBS or equicenta™CTM in media was reapplied to their respective wells, and the stimulant reIL-1β (10 ng/ml) was applied to half of the wells (n=18 wells; 9 wells with PBS, 9 wells with equicenta™CTM) and all explants were cultured for an additional 48 hours. (4) After exposure to PBS or equicenta™CTM with or without reIL-1 for 48 hours, the supernatant was collected and stored at −80 °C until PGE2 and sGAG analysis. Explants were placed in formalin and subsequently processed for histomorphometric analysis.

FIG. 19 shows mean±SEM PGE2 concentrations (pg/ml) in supernatant from cartilage and synovial explants exposed to PBS (grey bars) or equicenta™CTM (green bars) with (checkered bars) or without (solid bars) reIL-1β. There was a respective 17.5- and 8.7-fold increase in PGE2 concentrations when cartilage (p=0.0005 and p=0.0022) or synovial explants (p=0.0031 and p=0.0026) were stimulated with reIL-1β. There was no significant difference in the PGE2 concentration of supernatant from cartilage explants between the PBS or equicenta™CTM exposed groups stimulated with (p=0.3083) or without reIL β (p=0.9997). There was also no significant difference in the PGE2 concentration of supernatant from synovial explants between the PBS or equicenta™CTM exposed groups stimulated with (p=0.8879) or without reIL β (p=0.9837). Note: y-axis on the left represents values for the unstimulated (without reIL-1β) groups and y-axis on the right represents values for the reIL-1β stimulated groups.

FIG. 20 shows mean±SEM soluble sGAG concentrations (ng/ml) in supernatant from cartilage and synovial explants exposed to PBS (first and third bars from the left) or equicenta™CTM (second and fourth bars from left) with (checker bars) or without (solid bars) reIL-1β. There was no significant difference in sGAG concentrations from supernatant of cartilage or synovium explants exposed to PBS or equicenta™CTM. While not statistically different, stimulation of cartilage explants with reIL-1β resulted in a 2-fold increase in sGAG concentrations. Subsequent analyses confirmed that equicenta™CTM alone can significantly increase sGAGs concentration in media supernatant. Note: y-axis on the left represents values for the unstimulated (without reIL-1β) groups and y-axis on the right represents values for the reIL-1β stimulated groups.

FIG. 21 shows mean±SEM soluble sGAG concentrations (ng/ml) in PBS+media (first and third bars) or equicenta™CTM+media (second and fourth bars) with (checker bars) or without (solid bars) reIL-1β. Higher concentrations of sGAG (*p<0.0001) were observed in equicenta™CTM groups than in PBS groups.

FIGS. 22A and 22B show (FIG. 22A) Histological (10×, bar=100 um) images from cartilage explants exposed to PBS or equicenta™CTM stimulated with and without reIL-1β. sGAG staining (purple) by Toluidine blue is noted in the deeper cartilage zones. Lack of sGAG staining (black, double arrow) in the cartilage explants was noted in all groups in the superficial and outer intermediate zone, with some explants demonstrating, subjectively, loss into the inner intermediate zone (PBS+reIL-1l). There was also a subjective peri-territorial loss of sGAG noted (white large arrow) in the inner intermediate and deep zones of cartilage. (FIG. 22B) Objectively, the total GAG intensity score did not differ significantly between PBS (first bar) or equicenta™CTM (second bar) exposed groups (p=0.3139) stimulated with (checker bars) or without (solid bars) reIL-1(3 (p=0.4444).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. DEFINITIONS

It is understood that the disclosed method and compositions are not limited to the particular methodology, protocols, and reagents described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a composition” includes a plurality of such compositions, reference to “the composition” is a reference to one or more compositions and equivalents thereof known to those skilled in the art, and so forth.

The word “or” as used herein means any one member of a particular list and also includes any combination of members of that list.

As used herein, the term “umbilical cord” refers to the combination of blood vessels (veins and arteries) and amnion/amnion membrane and umbilical tissue that connects the developing fetus to the placenta. In some aspects, the umbilical cord does comprise an outer layer of amnion as an amniotic membrane that can be present as an outer layer of the umbilical cord. This outer amnion layer may serve to protect the umbilical tissue from contamination or trauma. In some aspects, the umbilical tissue can comprise a variable portion the thin encasing amnion if the amnion is not completely removed when processing the umbilical cord. In some aspects, a variable portion of amnion can be <10%, 9%, 8%, 7%, 6%, 5%, 4%, 3%, 2%, 1%, or 0.5% of the disclosed FDUT is amnion. In some aspects, a variable portion of amnion can be about 0-50% of the disclosed FDUT is amnion. In some aspects, the disclosed methods of making FDUT are designed to remove the amnion, which is an extremely thin and low-mass layer compared to the bulk of the umbilical cord. Consequently, any post-processing remnants would be negligible and fall well within a 0-2% threshold by weight.

In placental mammals, the umbilical cord (UC) connects the developing fetus to the placenta and typically comprises two umbilical arteries and one umbilical vein embedded within a hydrated connective-tissue matrix. The cord may be externally bounded by an amniotic membrane (amnion).

As used herein, “umbilical tissue” (UT) means the parenchymal connective-tissue portion of the UC excluding the external amniotic membrane. UT may include some or all vascular structures present in the cord (e.g., the umbilical vein and arteries, including their vessel walls and luminal contents), as they occur in situ. In some aspects, umbilical tissue is derived from umbilical cord. UT contains connective tissue substances in a loose matrix network that can be both structural and supplemental to natural trophic and anti-inflammatory processes.

In some aspects, umbilical tissue (UT) comprises mesenchymal stromal cells (MSCs) residing within the parenchymal connective-tissue matrix of the umbilical cord.

In some aspects, UT is substantially free of epithelial cells because UT excludes the external amniotic membrane (amnion).

As used herein, “Fillet-derived umbilical tissue” or “FDUT” means the non-vascularized parenchymal connective-tissue matrix obtained from the fillet portion of the umbilical cord excluding both (i) the external amniotic membrane (amnion) and (ii) the large internal vessels (the umbilical vein and umbilical arteries, including their vessel walls and luminal contents). FDUT is debulked of the large vessels and is substantially free of patent vascular lumens. Due to mechanical dissection and cleaning tolerances, FDUT may contain trace residual amniotic membrane in an amount of about 0-2% by weight (≤2 wt %) of the FDUT, calculated on a wet-weight basis unless stated otherwise.

In some aspects, FDUT is substantially free of endothelial cells because the transition of UT to FDUT involves the excising of blood vessels (vein and arteries, including their vessel walls and luminal contents) to provide a parenchymal matrix free of the large blood vessels to the foal. FDUT may contain trace residual endothelial cells in an amount of about 0-2% by weight (≤2 wt %) of the FDUT, calculated on a wet-weight basis unless stated otherwise.

As used herein, the term “treating” refers to partially or completely alleviating, ameliorating, relieving, delaying onset of, inhibiting progression of, reducing severity of, and/or reducing incidence of one or more symptoms or features of a particular disease, disorder, injury, and/or condition. In some aspects, “treating” refers to the action or way of treating a patient or a condition medically or surgically: management and care to prevent, cure, ameliorate, or slow progression of a medical condition. For example, “treating” a tissue may refer to inhibiting or blocking the worsening of a wound. Treatment may be administered to a subject who does not exhibit signs of a disease, disorder, injury, and/or condition and/or to a subject who exhibits only early signs of a disease, disorder, injury, and/or condition for the purpose of decreasing the risk of developing pathology associated with the disease, disorder, injury, and/or condition.

As used herein, “subject” refers to the target of administration, e.g. an animal. Thus, the subject of the disclosed methods can be a vertebrate, such as a mammal. For example, the subject can be a horse. The term does not denote a particular age or sex. Subject can be used interchangeably with “individual” or “patient”.

As used herein, “native” refers to compositions or materials that are native, resident, or endogenous to the tissue sample, i.e. cells that are not exogenously added to the fillet-derived equine umbilical tissue. For example, FDUT contains a protein rich collagen matrix that is “native to” the fillet-derived equine umbilical tissue that is resident or endogenous to the umbilical tissue from which it is obtained.

“Native cells” means cells that are native, resident, or endogenous to the tissue sample, i.e. cells that are not exogenously added to the fillet-derived equine umbilical tissue.

“Native factors” means factors that are native, resident, or endogenous to the tissue sample, i.e. factors that are not exogenously added to the fillet-derived equine umbilical tissue.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### B. COMPOSITIONS

In placental mammals, the umbilical cord connects the developing fetus to the placenta. The umbilical cord consists of veins and arteries (usually one vein and two arteries) embedded in hydrated connective-tissue matrix (i.e. umbilical tissue) which can be encased in amnion (also known as amnion layer or amnion membrane”). The veins and arteries are the umbilical blood vessels. In some aspects, UT may still include the internal vessels, thus it can be free of epithelial cells (from amnion) but may contain endothelial cells (from vessels). The native components of umbilical tissue (UT) include endogenous cells, extracellular matrix (ECM), and bioactive factors. Endogenous cells found in UT include stromal cells, such as fetal and neonatal fibroblasts, myofibroblasts, mesenchymal stem cells, and macrophages. In some aspects, because the amnion acts as an outer layer or lining to the umbilical cord, when processing umbilical tissue from an umbilical cord a small percentage of amnion and amniotic cells (some epithelial cells) may be present in the umbilical tissue. In some aspects, the disclosed methods of making the disclosed compositions are designed to remove the amnion, which is an extremely thin and low-mass layer compared to the bulk of the umbilical cord. Consequently, any post-processing remnants would be negligible and fall well within a 0-2% threshold by weight.

In some aspects, amniotic epithelial cells, although not part of the umbilical tissue, can be added to the disclosed compositions. Thus, disclosed are compositions comprising fillet-derived umbilical tissue and amniotic tissue and/or cells.

Disclosed are compositions comprising fillet-derived umbilical tissue (FDUT). As used herein, the term “fillet” or “fillet-derived portion” of the umbilical cord refers to the segment of the umbilical cord extending from the fetal insertion point up to the point where the umbilical vein branches into two or more vessels. In some aspects, the umbilical cord does include the outer amnion that covers the umbilical cord and the fillet or fillet-derived portion of the umbilical cord may comprise amnion. In some aspects, FDUT is UT that has been de-vesselled (large vein/arteries removed), yielding a non-vascularized matrix substantially free of both epithelial and endothelial cells. In some aspects, FDUT may contain trace amnion (~0-2 wt % wet basis) due to mechanical tolerances. Thus, in some aspects, FDUT is a processed portion of the UT.

Thus, in some aspects, the fillet-derived portion is derived from the segment of the umbilical cord extending from the fetal insertion point up to the point where the umbilical vein branches into two or more vessels. In some aspects, the fillet-derived portion is enveloped or externally lined by the amnion.

In some aspects, the branching point (where the umbilical vein branches into two or more vessels) can occur anywhere from 5 cm to over 30 cm from the fetal end. In some aspects, the branching point occurs near the midpoint of the umbilical cord. Thus, in some aspects, the fillet-derived equine umbilical tissue is derived from a portion of equine umbilical cord located closest to the foal during pregnancy and extending out about 5, 10, 15, 20, 25, 30, 35, 40, 45, 50, 55, 60, 65, 70, 75, 80, 85, 90, 95, or 100 cm. In some aspects, the presence of vascular branching (i.e., the branching point) can serve as an anatomical landmark for delineating the fillet region.

In some aspects, a fillet-derived umbilical cord comprises an umbilical tissue portion which is a collagen-, protein-, and/or glycan-rich matrix. In some aspects, the FDUT portion of the UT comprises collagen and glycosaminoglycans native to the FDUT portion. In some aspects, the FDUT portion is native to the umbilical cord. In some aspects, the fillet-derived umbilical tissue is composed of elements that are all from the same subject.

In some aspects, the FDUT portion comprises the outer layer of intervascular and the inner layer of perivascular connective tissue. In some aspects, the FDUT is what remains after removal of the outer amnion of the umbilical cord and the vessels. In some aspects, an outer layer of intervascular connective tissue refers to the intermediate stromal zone that fills the space between the umbilical vessels and sits just beneath the sub-amnionic surface. In some aspects, this region contains a relatively loose weave of type I/III collagen fibers embedded in a hyaluronic-acid-rich ground substance and harbors a moderate density of stellate stromal cells. In some aspects, a perivascular connective tissue refers to the concentric sheath that lies immediately adjacent to the area similar to the tunica media of each artery and vein. In some aspects, histologically shows the densest collagen bundle architecture, the highest proteoglycan content, and the greatest concentration of mesenchymal stromal cells, functioning as a pseudo-adventitia that cushions and stabilizes the vessels.

In some aspects, the fillet-derived umbilical tissue do not comprise tissue from a branch portion or any tissue that extends past the branch portion of an equine umbilical cord. Thus, in some aspects, the fillet-derived umbilical tissue comprise only umbilical tissue present from the fetal insertion point up to the point where the umbilical vein branches into two or more vessels.

In some aspects, the fillet-derived umbilical tissue is soft, supple, spongy, and compressible. In some aspects, “soft, supple, spongy, and compressible” refers to the lower density and greater malleability of the fillet-derived umbilical tissue compared to other regions such as the branch. In some aspects, the fillet-derived umbilical tissue feels softer and more pliable to the touch, with a texture that is more easily compressed. In some aspects, the soft, supple, spongy, and compressible texture of the fillet-derived umbilical tissue is compared to the branch tissue (e.g. tissue that begins at the branching point) which is denser, firmer, and more resistant to deformation. In some aspects, a histological difference of the fillet-derived umbilical tissue corresponds to a looser, hydrated extracellular matrix (ECM) with fewer tightly packed collagen fibers compared to the tissue at or following (away from the fetal attachment point) the branch point. In some aspects, these characteristics of the fillet-derived umbilical cord make it more conducive to dissociation during processing. In some aspects, fillet-derived umbilical tissue can be about 60-80% water. Of the dry weight, most can be fibrillar proteins (collagens, etc.), and a smaller portion can be glycans or bioactive proteins, like alpha 2 macroglobulin and proteoglycans.

In some aspects, the fillet-derived umbilical tissue comprises viable cells native to the connective tissue portion. In some aspects, viable cells native to the connective tissue portion refers to cells that are present in the naturally occurring umbilical tissue. In some aspects, viable cells can be seeded onto the fillet-derived umbilical tissue. Thus, in some aspects, the viable cells can be derived from cells native to a connective tissue portion but that have been cultured in vitro prior to seeding on the fillet-derived equine umbilical tissue. In some aspects, the viable cells can be mesenchymal stem cells, fibroblasts, or a combination thereof. In some aspects, the fillet-derived umbilical tissue comprises 70% native viable cells. In some aspects, the fillet-derived umbilical tissue comprises at least 5%, 10%, 15%, 20%, 25%, 30%, 35%, 40%, 45%, 50%, 55%, 60%, 65%, 70%, 75%, 80%, 85%, 90% or 95% native viable cells. In some aspects, the fillet-derived umbilical tissue comprises at least 70% native viable cells means that there are at least 70% native viable cells in the fillet-derived umbilical tissue compared to the number of viable cells in the fillet portion of the UC prior to processing of the umbilical tissue. For example, the umbilical tissue prior to processing can have 10%, 20%, 30%, 40%, 50%, 60%, 70%, 80%, 90%, or 100% of viable cells in the fillet-derived umbilical tissue from native umbilical cord. Thus, the disclosed compositions comprising a fillet-derived umbilical tissue comprise at least 70% native viable cells means that the compositions comprise 70% of viable cells from the fillet portion of the umbilical tissue prior to processing of the umbilical tissue and not necessarily 70% of viable cells in the fillet-derived umbilical tissue compared to the fillet portion of native umbilical tissue. In an aspect, the native viable cells are cells that are native to the fillet portion of the original umbilical tissue (e.g. the umbilical tissue used in the methods described herein) and have not been removed from the tissue. For example, native viable cells are not cells that have been seeded on or in the fillet-derived umbilical tissue of the umbilical tissue from the same or a different source.

In some aspects, the fillet-derived umbilical tissue comprises exosomes native to the fillet-derived umbilical tissue. Exosomes are small extracellular vesicles (~30-150 nm) that form as intraluminal vesicles inside multivesicular endosomes and are released when those endosomes fuse with the plasma membrane. In some aspects, exosomes are enclosed by a lipid bilayer and carry proteins, lipids, and nucleic acids reflective of their parent cell. In some aspects, exosomes are identified by the presence of at least one tetraspanin surface marker: CD63, CD9, or CD81. In some aspects, CD9-only exosomes account for approximately 50-70% of the total particles in the fillet-derived umbilical tissue. In some aspects, CD9+ exosomes account for about 90% or more of the exosomes with co-expressed markers. in the fillet-derived umbilical tissue In some aspects, about 10-15% of exosomes can be CD81-positive. In some aspects, more exosomes express CD63 compared to CD9 or CD81. In some aspects, the exosomes can co-express CD9 and CD81. In some aspects, the exosomes can co-express CD9 and CD63. In some aspects, the exosomes can co-express CD81 and CD63. In some aspects, the exosomes can co-express CD9, CD81 and CD63. In some aspects, the presence of CD105, CD90, and CD73—markers associated with stem-cell membranes—can be used to identify exosomes present in the fillet-derived umbilical tissue. In some aspects, one or more of CD105, CD90, or CD73 can be present on the exosome in combination with one or more of CD9, CD81 or CD63.

In some aspects, the fillet-derived umbilical tissue comprises a profile of its own native growth factors. In some instances, the growth factors can be, but are not limited to, fibroblast growth factor (FGF), epidermal growth factor (EGF), hepatocyte growth factor (HGF), keratinocyte growth factor (KGF), basic fibroblast growth factor (bFGF), TGF-β1, 2, and 3, insulin-like growth factor-1 (IGF-1), vascular endothelial growth factor (VEGF), VEGF-C, VEGF-D, TGF-α, Interleukin 10 (IL-10), Interleukin—1 receptor α (IL-1rα), Stromal cell-derived factor-1 (SDF-1), Basic fibroblasts growth factor (bFGF), Neutrophil gelatinase-associated lipocalin (N-Gal), Matrix metalloproteinase 8 (MMP8), Tissue inhibitor of metalloproteinase 1 (TIMP1), TIMP2, Angiopoietin 2 (hAng2), thrombospondin 2 (TSP2), Platelet derived growth factor AA (PDGF-AA), PDGF-AB, Placental growth factor (PIGF), Insulin-like growth factor (IGFBP1), IGFBP2, IGFBP3, α2-macroglobulin, Adiponectin (hACRP30), Fibronectin, Interferon gamma (IFN-γ), IL-1α, Monocyte Chemoattractant Protein-1 (MCP-1) or a combination thereof. In some aspects, the fillet-derived umbilical tissue comprises IGF, A2M, and TIMP-2. In some aspects, the fillet-derived umbilical tissue are capable of releasing cytokines, growth factors, and protease inhibitors, such as, but not limited to, IGF, A2M, and TIMP-2.

In some aspects, a profile of functional proteins comprises one or more of metalloproteinases-2 (TIMP-2), tissue inhibitor of metalloproteinases-1 (TIMP-1), and alpha-2-macroglobulin (A2M). IN some aspects, TIMP-2 increases relative to an unextracted or undigested baseline following enzymatic digestion of the FDUT. In some aspects, the composition can be characterized by anti-protease activity, wherein TIMP-2, A2M, and/or TIMP-1 are present at high levels.

In some aspects, a profile of native growth factors comprises a pro-regenerative payload including IGF-1, bFGF, and TGF-β1. In some aspects, anabolic growth factors increase upon extraction, detergent extraction, or enzymatic digestion relative to an unextracted or undigested baseline, while pro-inflammatory cytokines, including IL-1α and IL-8, do not increase.

In some aspects, release of the native growth factors is biphasic, comprising (i) an immediate soluble fraction and (ii) a sustained fraction released from an extracellular-matrix network during biodegradation with native enzymes.

In some aspects, the fillet-derived umbilical tissue comprises cytokines native to the FDUT of naturally occurring umbilical tissue. In some aspects, the cytokines can be, but are not limited to, interferon gamma (IFNg), interleukin-1 (IL-1), interleukin-1 alpha (IL-1 alpha), interleukin-1 receptor antagonist (IL-1ra), interleukin-2 (IL-2), interleukin-4 (IL-4)., interleukin-8 (1L-8), interleukin 10 (IL-10), interleukin-15 (IL-15), monocyte chemoattractant protein-1 (MCP-1) or a combination thereof.

In some aspects, the fillet-derived umbilical tissue can comprise other components that can assist with the resistance to compression in the umbilical cord. In some aspects, the fillet-derived umbilical tissue comprises hyaluronic acid, a hexose sugar that is known to provide compressibility.

In some aspects, the fillet-derived umbilical tissue is partially devascularized. Thus, the disclosed compositions can be devoid of most, but not all, arteries or veins present in the umbilical tissue. For example, the disclosed fillet-derived umbilical tissue can be devoid of the two arteries and one vein typically found in the umbilical tissue. In some aspects, some of the smaller arteries can remain. In some aspects, the partial devascularization is not a chemical devascularization.

In some aspects, the fillet-derived umbilical tissue does not comprise amnion or amnion-derived components. Thus, in some aspects, the fillet-derived umbilical tissue is not derived from an outer membrane amnion that covers or externally lines the umbilical cord. In some aspects, the fillet-derived umbilical tissue has the connective tissue structure of a fillet portion of an umbilical cord devoid of amnion. Thus, in some aspects, the connective tissue structure of the fillet-derived umbilical tissue is identical to the connective tissue structure of the fillet portion of an umbilical cord in its native state (e.g. naturally occurring). In some aspects, during processing of an umbilical cord to produce the fillet-derived umbilical tissue, the inherent structural integrity of the connective tissue, meaning its collagen network, ground substance, and cellular organization, remains essentially in its natural configuration, as found in the native umbilical tissue.

In some aspects, the fillet-derived umbilical tissue comprises functional proteins native to the fillet-derived umbilical tissue. Thus, in some aspects, the removal of the umbilical tissue from the subject and any further processing (e.g., removal of blood vessels) does not alter the presence of functional proteins in the umbilical tissue, specifically, the presence of functional proteins in the fillet-derived umbilical tissue. For example, proteins found in naturally occurring umbilical tissue can remain as functional proteins in the fillet-derived umbilical tissue. In some aspects, the functional proteins can be, but are not limited to, Chondroitin sulfate proteoglycan, Biglycan Proteoglycan in ECM, Asporin, Versican Core Protein, Collagen type 1 alpha, Four smaller collagens in ECM, Superoxide dismutase, Annexin Al, Heat Shock Protein 90, Galectin, Tenascin, Integrin Subunit alpha (3), Calpain, Proline and arginine rich end leucine rich repeat protein, Alpha 2 macroglobulin, Heparan sulfate proteoglycan 2, Annexin (lipocortin), Decorin, Transgelin, Vinculin, Collagens VI, XII, IV, Thrombospondin, Profilin, Tubulin, Actinin, TGF-β induced protein ig-h3, Lumican, Plectin, or any combination thereof. In some aspects, functional proteins can be, but are not limited to, one or more of those listed in Table 1 or 2.

In some aspects, the fillet-derived umbilical tissue comprises no more than about 5% of native blood cells from the initial fillet-derived umbilical tissue (prior to processing). For example, the fillet-derived umbilical tissue comprises no more than about 5% of native blood cells found in native or naturally occurring umbilical tissue. In some aspects, the fillet-derived umbilical tissue comprises no more than about 10%, 9%, 8%, 7%, 6%, 5%, 4%, 3%, 2%, or 1% of native blood cells from the initial fillet-derived equine umbilical tissue. In some aspects, the fillet-derived umbilical tissue comprises an amount of blood cells that does not trigger an immune reaction and allows for improved biocompatibility and reduced immunogenicity. In some aspects, the absence or decreased amounts of blood cells can be due to the removal of blood vessels and capillaries and/or washing or rinsing the fillet-derived umbilical tissue. In some aspects, one or more anticoagulants can be used to help remove blood and blood products, for instance ACD-A (anticoagulant Citrate dextrose solution, solution A, USP), heparin, dalteparin sodium, and bivalirudin. If anticoagulants are used, the fillet-derived equine umbilical tissue can be washed so that the anticoagulant is not present in the final product. In some aspects, native blood cells refers to all blood cells including white blood cells, red blood cells, and platelets.

In some aspects, the fillet-derived umbilical tissue is dissociated into pieces less than 500 μm, less than 300 μm, less than 200 μm, less than 100 μm, less than 50 μm, less than 10 μm, less than 5 μm, less than 1 μm. In some aspects, the fillet-derived equine umbilical tissue is minced, micronized, chopped, shredded, or homogenized. In some aspects, the fillet-derived equine umbilical tissue is minimally manipulated to dissociate the fillet-derived equine umbilical tissue. In some aspects, for structural tissues, minimally manipulated refers to processing that does not alter the original relevant characteristics of the tissue relating to the tissue's utility for reconstruction, repair, or replacement. In some aspects, for non-structural tissues or cells, it is defined as processing that does not alter the relevant biological characteristics of cells or tissues. In some aspects, the fillet-derived umbilical tissue is mechanically dissociated.

In some aspects, the disclosed compositions are comprised of a suspension derived from about 50 mg fillet-derived equine umbilical tissue/1 ml total volume. In some aspects, the disclosed compositions are comprised of a suspension derived from about 1-250 mg fillet-derived umbilical tissue/1 ml total volume. In some aspects, the disclosed compositions comprise protein cytokine concentrations about a range of 0 to 2,500 μg/ml from a 50 mg fillet-derived umbilical tissue protein/1 ml total volume. In some aspects, about 20% of tissue weight can be lost during processing. In some aspects, the composition is not in solution but rather can be in a dry or solid form. Thus, in some aspects, the concentrations provided herein can be by volume or by weight.

In some aspects, the compositions disclosed herein can be in a flowable solution. In some aspects, a flowable solution can also be referred to as a flowable microparticulate suspension. In some aspects, “flowable solution” can refer to a composition being in a liquid or semi-liquid state suitable for administration via standard syringe-based delivery, including, but not limited to, through narrow-gauge or typical-gauge needles such as an 18 or 20 gauge needle. In some aspects, “flowable solution” or “flowable suspension” can refer to a composition having a low enough viscosity and appropriate particulate size to be delivered without clogging a delivery device. In some aspects, the disclosed compositions being in a flowable solution allows for injection into tissue and not necessarily just an external cover of a wound. In some aspects, a flowable solution allows for deeper penetration into wound beds or soft tissue defects, supporting internal healing and enhancing integration with host tissue.

In some aspects, the fillet-derived equine umbilical tissue retains all or a portion of the allantoic duct. In some aspects, the fillet-derived equine umbilical tissue does not retain the allantoic duct.

In some aspects, an example of one of the disclosed fillet-derived umbilical tissues can be referred to as equicenta™CTM.

### 1. Tissue Preservation Compositions

In some aspects, the fillet-derived umbilical tissue is cryopreserved, vitrified, lyophilized (freeze-dried), desiccated, glycerol-preserved, or supercritically dried, or has been previously preserved by one or more of these methods.

In some aspects, the fillet-derived umbilical tissue can undergo detergent/enzymatic decellularization to yield an acellular scaffold that can then be stored by various methods. In some aspects, the fillet-derived umbilical tissue can undergo chemical fixation/crosslinking (glutaraldehyde, EDC/NHS, genipin, etc.) to stabilize collagen and slow enzymatic degradation. In some aspects, the fillet-derived umbilical tissue can undergo radiation-based processing (gamma, e beam, UV) to sterilize and induce additional collagen crosslinking. In some aspects, the fillet-derived umbilical tissue can undergo peracetic acid or antibiotic-based terminal sterilization of decellularized tissue. In some aspects, the fillet-derived umbilical tissue can undergo sugar/polyol-based stabilization (e.g., trehalose, sucrose, non-glycerol polyols) to protect matrix during drying or freezing. In some aspects, the fillet-derived umbilical tissue can undergo polymer embedding or composite formation (e.g., milling the ECM and incorporating into hydrogels or solid scaffolds).

In some aspects, the disclosed compositions can further comprise a cryopreservation solution. In some aspects, the disclosed compositions can be previously cryopreserved. In some aspects, the disclosed compositions can be cryopreserved. In some aspects, “previously cryopreserved” can mean a composition that was once cryopreserved but has been removed (e.g., thawed) from the cryopreserved state.

In some aspects, a cryopreservation solution can contain one or more non-cell permeating cryopreservatives. Examples of non-cell permeating cryopreservatives, include but not limited to, polyvinyl pyrrolidione, a hydroxyethyl starch, a polysaccharide, a monosaccharide, an alginate, trehalose, raffinose, dextran, human serum albumin, Ficoll, lipoproteins, hydroxyethyl starch, autologous plasma, or a mixture thereof. In some aspects, the cryopreservative comprises DMSO or glycerol. Further, a cryopreservation solution can contain serum albumin or other suitable proteins to stabilize the disclosed compositions during the freeze-thaw process and to reduce the damage to cells, thereby maintaining viability. In some aspects, a cryopreservation solution can contain a physiological solution, such as a physiological buffer or saline, for example phosphate buffer saline. In some aspects, a cryopreservation solution can comprise a lyoprotectant, such as trehalose or trehalose in combination with one or more antioxidants.

In some aspects, the disclosed compositions can further comprise a lyopreservation agent. In some aspects, the disclosed compositions can be previously lyopreserved. In some aspects, the disclosed compositions can be lyopreserved. In some aspects, “previously lyopreserved” can mean a composition that was once lyopreserved but has been removed (e.g., reconstituted) from the lyopreserved state.

In some aspects, the lyophilized fillet-derived umbilical tissue disclosed herein can be reconstituted resulting in a reconstituted fillet-derived umbilical tissue (or previously lyophilized fillet-derived umbilical tissue). Lyophilized fillet-derived umbilical tissue can be reconstituted using standard techniques known in the art. In some aspects, reconstituting refers to rehydrating. Thus, the disclosed lyophilized fillet-derived umbilical tissue can be reconstituted or rehydrated using water, saline, a buffer such as, but not limited to phosphate buffered saline (PBS), in a solution comprising a stabilizing agent such as, but not limited to bovine serum albumin (BSA), Plasma-Lyte A or other clinically available electrolyte solutions, blood plasma, blood derivatives with a bodily fluid or a combination thereof. For example, lyophilized fillet-derived umbilical tissue can be applied directly to a wound or tissue injury on a subject and the subject's bodily fluids can reconstitute. In some aspects, a combination of bodily fluids and another known rehydrating solution can be used. Also, disclosed are lyophilized or reconstituted fillet-derived umbilical tissues prepared using the methods disclosed herein.

In some aspects, the disclosed lyophilized fillet-derived umbilical tissue comprises less than 15% residual water. In some aspects, the disclosed lyophilized fillet-derived umbilical tissue comprises 5-12% residual water. In some aspects, the disclosed lyophilized fillet-derived umbilical tissue comprises≤5% residual water.

In some aspects, the disclosed lyophilized compositions or lyophilized fillet-derived umbilical tissue comprises trehalose. In some aspects, the disclosed lyophilized compositions or lyophilized fillet-derived umbilical tissue comprises trehalose, wherein the trehalose is present at a concentration of 0.25M-1.5M.

In some aspects, the lyophilized compositions or lyophilized fillet-derived umbilical tissue disclosed herein can be stable for at least three weeks. In some aspects, the lyophilized compositions or lyophilized fillet-derived umbilical tissue can be stable for at least three months. In some aspects, lyophilized compositions or lyophilized fillet-derived umbilical tissue can be stable for at least 1, 2, 3, 4, 5, 6, 7, 8, 9, 10, 11, 12, 24, 36, 48, or 60 months. In some aspects, lyophilized compositions or lyophilized fillet-derived umbilical tissue can be stable for at least 1, 2, 3, 4, 5, 6, 7, 8, 9, or 10 years.

### C. Methods of Treating

In the disclosed methods, the fillet-derived umbilical tissue (FDUT) in the composition disclosed herein can be derived from the same species as the subject being treated. Thus, the donor (from where the FDUT is derived) and the recipient (subject being administered the disclosed compositions) can be from the same species (allogeneic) or different species (xenogeneic).

Disclosed are methods of treating a condition in a mammal, the method comprising administering an effective amount of any one of the compositions disclosed herein to a target site in the mammal.

In some aspects, the condition is selected from the group consisting of a musculoskeletal condition, a tissue defect, pain, and inflammation. In some aspects, the musculoskeletal condition can be arthritis, arthrosis (osteoarthritis), desmitis, tendonitis, osteoarthropathy, desmopathy or tendonopathy. In some aspects, the tissue defect is a cutaneous wound or sub cutaneous wound.

Disclosed are methods of treating a musculoskeletal condition in a subject comprising administering an effective amount of one or more of the compositions disclosed herein to a target site selected from an articular joint, tendon, or ligament. In some aspects, the subject is a horse. Thus, disclosed are methods of treating an equine musculoskeletal condition comprising administering an effective amount of one or more of the compositions disclosed herein to a target site selected from an articular joint, tendon, or ligament.

In some aspects, treating comprises rejuvenating or restoring atrophied tissue or poor-quality healed tissue, by improving structural matrix integrity and/or cellular composition at the target site.

In some aspects, the target site is an articular joint, tendon, ligament, or skin. In some aspects, the skin can be a dermal layer or a sub-dermal layer.

In some aspects, administering the composition reduces catabolic enzyme activity and promotes tissue repair.

In some aspects, administering the composition results in the release of soluble glycosaminoglycan (sGAG) which can provide protective benefits in horses with a joint injury or osteoarthritis.

In some aspects, treating a horse with one or more of the compositions disclosed herein can result in improved lameness, pain, swelling and performance scores over time. In some aspects, the improvement is compared to a horse that is not treated with one or more of the compositions disclosed herein. In some aspects, the improvement is compared to a horse that is treated with the standard of care, which can include commercially available biologic therapies

In some aspects, the mammal is a human or an equine.

In some aspects, the composition is derived from a donor and the mammal receiving the composition is a recipient. In some aspects, the donor and recipient are of the same species (allogeneic) or different species (xenogeneic).

Disclosed are methods of treating tissue defects comprising administering one or more of the compositions disclosed herein to a subject in need thereof. For example, disclosed are methods of treating tissue defects include administering a composition comprising fillet-derived equine umbilical tissue to a subject in need thereof. In some aspects, administering can be performed intra-lesionally, intra-articularly, subcutaneously, intra-peritoneally, intraoperatively, intravenously, intra-arterially, topically, via nebulization, and/or via body cavity such as orally.

In some aspects, a tissue defect can be a wound (or injury). In some aspects a wound can be a cutaneous wound or a sub-cutaneous wound (e.g. a laceration, abrasion, or surgical site). thus, disclosed are methods of treating wounds. In some aspects, a wound can be, but is not limited to, an acute, a chronic, a surgical, or a traumatic wound. In some aspects, a wound can be, but is not limited to, ulcers (e.g., pressure ulcers, venous ulcers, arterial ulcers), burns, infected wounds, non-healing wounds, degloving injuries, soft tissue defects, summer sores, corneal injuries, or ischemic wounds. In some aspects, the disclosed methods can be used in wound healing. For example, for a surgical wound, the disclosed methods can be used in post-surgical healing to promote tissue regeneration and reduce inflammation.

In some aspects, disclosed are methods of promoting post-surgical healing wherein the disclosed compositions can be administered (e.g., injected) as an adjunct to surgical closure by applying intraoperatively to tissue beds. Disclosed are methods of promoting healing at a surgical site of a subject comprising administering one or more of the disclosed compositions during or immediately following a surgical procedure to the open surgical site or closed surgical site. For example, in three horses (four stifle joints), fillet-derived umbilical tissue suspension (1.5 ml) can be topically applied and allowed to tesselate into a surgically debrided osteochondral defect on the lateral trochlear ridge of the stifle joint at arthroscopic surgery. In some aspects, at 30 days following the application, horses can show clinical improvement. In some aspects, at 120 days horses can be determined to be sound. Blood can be analyzed from a horse after administration and after surgery for the presence of DMSO (the cryopreservative). In some aspects, no DSMO can be detected. In some aspects, a tissue defect can be a defect or injury to a subject's soft tissue. In some aspects, a soft tissue defect can be, but is not limited to, a tendon or ligament injury, or a defect in the skin, synovial tissue, or connective tissue. In some aspects, a soft tissue defect can be, but is not limited to, muscle injuries (e.g., strains, tears, contusions, volumetric loss), fascial defects (e.g., fascial tears, hernias), cartilage defects (e.g., articular cartilage defects, meniscal tears), nerve damage (e.g., peripheral nerve injuries), adipose tissue defects (e.g., lipoatrophy, for soft tissue augmentation), joint capsule injuries, or synovial membrane injuries.

In some aspects, a tissue defect or tissue pathology can be a cartilage, bone, or osteochondral defect. In some aspects, tissue defects can occur following injury. In some aspects, tissue pathology can be scarring, failure to heal, inadequate healing, or replacement of injured tissue with inadequate amount or quality of tissue to sustain function. Such states may be termed osteoarthropathies, desmopathies, tendonopathies.

Thus, disclosed herein are methods of treating tissue defects comprising administering the composition of any one of the disclosed compositions to a subject in need thereof.

Disclosed are methods of treating a cartilage and/or bone lesion in a subject comprising administering one or more of the compositions disclosed herein to a subject in need thereof. For example, disclosed are methods of treating a cartilage and/or bone lesion in a subject by administering a composition comprising fillet-derived umbilical tissue to a subject having a cartilage and/or bone lesion. In some aspects, the composition can be administered by injection or surgical or topical application at surgery.

Disclosed are methods of treating pain in a subject comprising administering one or more of the compositions disclosed herein to a subject in need thereof. For example, disclosed are methods of treating pain in a subject by administering a composition comprising fillet-derived umbilical tissue to a subject having pain. In some aspects, the pain can be joint pain or ligament pain. In some aspects, the pain can be from arthritis or an injury.

Disclosed are methods of treating inflammation in a subject comprising administering one or more of the compositions disclosed herein to a subject in need thereof. For example, disclosed are methods of treating inflammation in a subject comprising administering a composition comprising fillet-derived equine umbilical tissue to a subject with inflammation. In some aspects, the inflammation can be in a joint or ligament. In some aspects, treating a joint can comprise diarthrodial or a synostotic joints. In some aspects, the inflammation can be from arthritis or an injury. Thus, in some aspects, disclosed are methods of treating arthritis (e.g., osteoarthritis), desmitis, tendonitis, arthrosis, osteoarthrosis, osteoarthropathy, tendinosis, tendonopathy, desmosis, and/or desmopathy in a subject (e.g., a horse) comprising administering one or more of the compositions disclosed herein to a subject in need thereof. In some aspects, the inflammation can be pulmonary inflammation, including but not limited to immune mediated pulmonary disease.

Disclosed are methods of treating inadequate healing and scarring. For example, disclosed are methods of treating inadequate healing and scarring in a subject by administering a composition comprising fillet-derived umbilical tissue to a subject having inadequate healing and scarring. Chronic injury can be non-inflammatory, but subject to reinjury upon return to use due to insufficient mechanical strength for the use. This represents the rejuvenation aspect of a pro-regenerative product.

Disclosed are methods of treating corneal injuries, hoof wall injuries, heaves, and/or bleeders comprising administering one or more of the compositions disclosed herein to a subject in need thereof. For example, disclosed are methods of treating corneal injuries, hoof wall injuries, heaves, and/or bleeders in a subject by administering a composition comprising fillet-derived umbilical tissue to a subject having a corneal injury, hoof wall injury, heaves, and/or bleeders. Methods of administration may include topical and nebulization.

Disclosed are methods of treating a musculoskeletal condition comprising administering an effective amount of any one of the compositions disclosed herein to a target site selected from an articular joint, tendon, or ligament. For example, disclosed are methods of treating a musculoskeletal condition in a subject by administering a composition comprising fillet-derived umbilical tissue to a subject having a musculoskeletal condition. In some aspects, the musculoskeletal condition is an equine musculoskeletal condition.

Disclosed are methods of rejuvenating and restoring atrophied or poor quality healed tissue defects comprising administering any one of the disclosed compositions to a subject in need thereof. For example, disclosed are methods of rejuvenating and restoring atrophied or poor quality healed tissue defects in a subject by administering a composition comprising fillet-derived umbilical tissue to a subject having a poor quality healed tissue defects. In some aspects, healed tissue may be quiet (not inflamed or painful) yet can inadequately support use, such as the horse returning to performance.

In some aspects, the composition used in any of the disclosed methods comprises a fillet-derived equine umbilical tissue.

In some aspects, administering the composition comprises direct administration of the composition to a joint or tissue defect of the subject in need thereof. In some aspects, administration is via a syringe or syringe-like device that allows delivery of a flowable solution.

In some aspects, the subject in need thereof is any mammal. In some aspects, the subject in need thereof is a horse.

### D. Methods of Making

Disclosed herein are methods of making the disclosed fillet-derived umbilical tissue (FDUT) or compositions comprising a FDUT.

Disclosed are methods of making or producing a fillet-derived umbilical tissue comprising obtaining or having obtained an umbilical cord having a fillet portion and a branch portion; separating the fillet portion of the umbilical cord and the branch portion of the umbilical cord; removing the branch portion of the umbilical cord; and removing an outer membrane from the fillet portion of the umbilical cord; thereby producing a fillet-derived umbilical tissue.

In some aspects of methods of making or producing a fillet-derived umbilical tissue, the fillet portion is still associated with the full umbilical cord tissue and the fillet-derived portion has been removed from full umbilical cord tissue. In some aspects, the fillet portion, similar to the fillet-derived portion, is the segment of the umbilical cord extending from the fetal insertion point up to the point where the umbilical vein branches into two or more vessels. In some aspects, the fillet portion is enveloped by the amnion.

In some aspects of methods of making or producing a fillet-derived umbilical tissue, the branch portion is the segment of the umbilical cord starting at the point where the umbilical vein branches into two or more vessels.

In some aspects of methods of making or producing a fillet-derived umbilical tissue, separating the fillet portion of the umbilical cord and the branch portion of the umbilical cord comprises cutting the umbilical cord at the anatomical branching point that delineates the fillet portion from the branch portion.

In some aspects of methods of making or producing a fillet-derived umbilical tissue, removing an outer membrane from the fillet portion of the umbilical cord comprises removing the amnion. In some aspects of methods or making or producing a fillet-derived umbilical tissue, removing the amnion, or amnion membrane layer surrounding the umbilical tissue, can be performed by simple mechanical dissection using a surgical instrument such as, but not limited to, a scalpel, scissors, and/or forceps.

In some aspects of methods of making or producing a fillet-derived umbilical tissue, the methods can further comprise removing blood vessels from the fillet portion of the umbilical cord. In some aspects, removing blood vessels from the fillet portion of the umbilical cord comprises dissection and/or scissors to cut open the vessels and then stripping or removing of the blood vessels. In some aspects, the process includes stripping of the inner vessel from the perivascular FDUT along natural tissue planes.

In some aspects of methods of making or producing a fillet-derived umbilical tissue, the methods can further comprise removing blood clots from the fillet portion of the equine umbilical cord. In some aspects, removing blood blots can comprise cutting them or physically removing them.

In some aspects of methods of making or producing a fillet-derived umbilical tissue, the methods can further comprise removing any unhealthy or low-viability regions of the umbilical cord (e.g., visibly bruised, necrotic, or otherwise compromised segments of the umbilical cord). In some aspects of methods or making or producing a fillet-derived umbilical tissue, the removal can be surgical removal/excision. In some aspects of methods or making or producing a fillet-derived umbilical tissue, excision of these low-viability regions ensures that the final fillet-derived umbilical tissue is enriched for healthy connective tissue while simultaneously removing residual blood elements and surface epithelial cells that could trigger an unwanted immune response in the recipient. In some aspects of methods or making or producing a fillet-derived umbilical tissue, after excision, the remaining tissue is rinsed/washed as described herein so that total native blood cells in the final composition remain below about 5% by count from the FDUT, further reducing immunogenic burden.

In some aspects of methods of making or producing a fillet-derived umbilical tissue, the methods can further comprise incubating the fillet portion of the umbilical cord with one or more antibiotics. In some aspects of methods or making or producing a fillet-derived umbilical tissue, the incubating with one or more antibiotics can be for a period of time to remove bacterial contamination. In some aspects of methods or making or producing a fillet-derived umbilical tissue, the removal of bacterial contamination helps maintain a sterile specimen. In some aspects of methods or making or producing a fillet-derived umbilical tissue, the incubating with one or more antibiotics can be for 10 min, 20 min, 30 min, 40 min, 50 min, 1 hr, 2 hr, 3 hr, 4 hr, 5 hr, 6 hr, 7 hr, 8 hr, 9 hr, 10 hr, 11 hr, 12 hr, 13 hr, 14 hr, 15 hr, 16 hr, 17 hr, 18 hr, 19 hr, 20 hr, 21 hr, 22 hr, 23 hr, or 24 hr. In some aspects of methods or making or producing a fillet-derived umbilical tissue, incubating with one or more antibiotics can be for at least 1 day, 2 days, 3 days, 4 days, or 5 days.

In some aspects of methods of making or producing a fillet-derived umbilical tissue, the methods can further comprise rinsing the fillet portion of the equine umbilical cord to remove the one or more antibiotics. In some aspects of methods or making or producing a fillet-derived umbilical tissue, rinsing can include soaking. In some aspects of methods or making or producing a fillet-derived umbilical tissue, the rinsing (or soaking) occurs with a solution that does not comprise an antibiotic. In some aspects of methods or making or producing a fillet-derived umbilical tissue, the rinsing step uses a saline solution. In some aspects, the saline is a sterile saline solution.

In some aspects of methods of making or producing a fillet-derived umbilical tissue, the methods can further comprise dissociating the fillet-derived umbilical tissue into pieces about 100 μm or smaller in size. In some aspects, the pieces are about 5 μm to 500 μm in size. In some aspects, the pieces are up to about 500 μm, 400 μm, 300 μm, 200 μm, or 100 μm in size. In some aspects, the pieces are at least about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, or 100 μm in size. In some aspects, the pieces are no larger than 100 μm in size. In some aspects, dissociating comprises mechanical dissociation. In some aspects, dissociating comprises manual dissociation using a scalpel or scissors. In some aspects, the size of the particles allows for both injectability and biocompatibility. In some aspects, the smaller particle sizes (e.g., less than 100 μm) can avoid triggering immune responses, likely due to the reduced surface area and lower risk of vascular or inflammatory irritation.

In some aspects of methods of making or producing a fillet-derived umbilical tissue, the methods can further comprise straining or filtering the pieces through a 500 μm, 300 μm, 200 μm, 100 μm, 50 μm, 10 μm, 5 μm, and/or smaller strain. In some aspects, straining can remove any larger, undesirable particulates or residual tissue that were not properly broken down during the dissociation process. In some aspects, straining comprises the use of a sterile mesh, screen, or sieve filters. In some aspects, only the particles/pieces that go through the strainer/filter are retained as the desired product. In some aspects, the straining or filtering process results in fillet-derived equine umbilical tissue pieces around 100 μm or smaller in size.

In some aspects of methods of making or producing a fillet-derived umbilical tissue, the methods can further comprise testing the fillet-derived umbilical tissue for residual microorganisms in the umbilical tissue. In some aspects, the methods are designed to result in the most sterile product possible that does not contain microorganisms. In some aspects, testing for residual microorganisms can be referred to as bioburden testing.

In some aspects of methods of making or producing a fillet-derived umbilical tissue, the methods can further comprise producing a final concentration from about 50 mg fillet-derived umbilical tissue/1 ml total volume. In some aspects, the concentration can be from about 20-100 mg fillet-derived umbilical tissue/1 ml total volume. In some aspects, the concentration can be from about 2-200 mg fillet-derived umbilical tissue/1 ml total volume. In some aspects, the concentration can be from about 5-500 mg fillet-derived umbilical tissue/1 ml total volume. In some aspects, the disclosed compositions comprise a final concentration of from about 250-1 mg fillet-derived umbilical tissue/1 ml total volume. In some aspects, the disclosed compositions comprise a final protein cytokine concentration of about 0-1,000,000 ng fillet-derived equine umbilical tissue cytokine protein/1 ml total volume. In some aspects, about 20% of tissue weight can be lost during processing. In some aspects, the composition is not in solution but rather can be in a dry or solid form. Thus, in some aspects, the concentrations provided herein can be by volume or by weight.

In some aspects of methods or making or producing a fillet-derived umbilical tissue, the methods can further comprise cryoprotecting or lyopreserving the dissociated pieces. In some aspects, the cryoprotecting or lyopreserving can occur prior to dissociation into small pieces.

In some aspects of methods or making or producing a fillet-derived umbilical tissue, the methods can further comprise performing a sterility test on the equine umbilical tissue. In some aspects, sterility testing can be performed prior to or after the preservation process (e.g., vitrification, desiccation, glycerol preservation). Performing sterility testing after preservation allows for the detection of any contamination introduced during the process. In some aspects, the sterility test confirms the umbilical tissue is free of microbial contamination

In some aspects of methods or making or producing a fillet-derived umbilical tissue, the methods can further comprise performing endotoxin testing. In some aspects, the endotoxin testing assesses a level of bacterial endotoxins. In some aspects, the endotoxin testing allows for a safety measurement prior to use. In some aspects, the methods can further comprise keeping the umbilical cord-derived tissue comprising a level of bacterial endotoxins equal to or less than 0.5 EU/mL.

### E. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising fillet-derived umbilical tissue. The kits also can contain solutions, pharmaceutically acceptable carriers, and/or devices for administration (e.g. syringes).

### EXAMPLES

### A. Example 1—Cryopreserved Equine Umbilical Tissue Allograft Characterization and Biocompatibility In Vivo in Musculoskeletal Tissues: A Controlled Study

### 1. Background

i. Musculoskeletal Focus

Musculoskeletal (MSK) abnormalities limit mobility and physical capacity in both humans and animals. An abundance of recently published work focuses on the MSK systems of joints, tendons, and ligaments. The rapid advancements of care in this space are, in part, due to the increasing health and mobility span of humans and animals, as well as the high expectations for care of human and equine athletes as a social obligation in connection with sports entertainment. Animal investigations have long been integral to the advancement in the care of MSK tissues.

The equine species particularly is recognized for a high frequency of MSK injury. Horses are a validated model for MSK health in humans, both in basic science and in clinical applications, due to the early adoption of biologic products as well as the substantial overlap in physical activities and medical care. Despite the rapid increase in the use of biologic tissue products for musculoskeletal (MSK) tissues in both human and equine athletes over the past decade, products that offer safety, quality, biocompatibility, convenience, and consistent characterization are still in the early stages of development.

ii. Biologic Tissue Transplants

The study of biologic tissue transplants is extensive and has resulted in the clinical application of many products in humans and horses, including various forms and enhancements of plasma, platelet-rich plasma, and bone marrow-derived plasma solutions, primarily for autologous injectable application in humans and horses. Autologous flowable products, while unlikely to produce reactions upon reapplication to the donor, have substantial limitations, such as inconvenient and time-consuming processing, donor site morbidity, variable product content, and often donor co-morbidities that reduce quality and effectiveness of product, such as older age and osteoarthritis (OA).

Tissue transplants such as intact grafts of articular cartilage, tendons, and ligaments have been used extensively for surgical repair of defects in these tissues. The grafts have routinely included both autologous and allogeneic tissue replacement and augmentation. Autologous whole tissue grafts are limited by donor site morbidity with potential dysfunction at the harvest site, a concern particularly in athletes and young patients. Fresh allogeneic grafts offer the advantages of living tissue and minimal degradation or alteration of the proteins, such as may occur with lyophilization, but carry the proven risk of disease transmission. Intact frozen allografts are subject to the effects of freezing tissue in bulk, in part due to limited penetration of cryoprotectants. Some great successes have been achieved with autologous and allogeneic grafts, including a long-standing bio-scaffold still in use today, bone grafts. Bone grafts are available for clinical application as autografts, fresh and frozen allografts, as well as particulate and processed bone to supplement structure and support function. While bone grafts have been quite successful, grafted articular cartilage or articular cartilage constructs have met with greater challenges, as have tendon and ligament defects not amenable to an intact tissue graft. Contributing factors include the slow natural repair of these tissues and the dense matrix required for the physical capacity to return to expected levels of function. Consequently, allograft supplements for articular cartilage, tendon and ligament have lagged in advancement to credible application. Many applications, however, do not require surgery or complete intact grafts; novel alternatives are needed to expand graft utility and to facilitate application in multiple sites.

iii. Micro-Particulate Bio-Scaffolds

Innovative processing techniques combined with imaging modalities have explored particulate grafts to serve as supplementation to tissue defects and provide the potential to visualize the flow of the graft into anatomic tissue gaps by video endoscopy or ultrasound. Novel creation of particulate biologic tissue grafts by physical processing could result in the refinement of nonchemical means to create a minimally manipulated biologic milieu while retaining connective tissue structure and functional proteins. With innovation, fresh particulate biologic tissue grafts could be suspended in biocompatible cryopreservation solution and cryopreserved for years.

iv. Tissue Sources for Musculoskeletal Supplementation

More recently, the development of flowable particulate biologic scaffolds as grafts to supplement tissues in joints, tendons, and ligaments is in its early stages. These biologic scaffolds offer the advantages of not being nondegradable or a foreign substance, such as polyacrylamides or xenografts, providing the ability to select a tissue source that aligns with the structural support and biocompatibility of the intended tissue target. The most investigated tissue sources for microparticulate allografts in any species are birth tissues, including maternal placental tissue (chorioallantois), fetal tissue (amnionic membrane and fluid), and umbilical cord. Umbilical cord blood has been tissue-banked in both humans and horses, but as an allogeneic transplant would require cellular processing and is not inherently a tissue scaffold. In humans, chorioallantois/amnionic membranes and umbilical cord have been processed and applied in combination (amnion-umbilical cord), consistent with human placental anatomy. Uniquely, placentation is diffuse in equine species and the fetal amnionic membrane and umbilical cord can be isolated separately and collected independently from the maternal placenta. Lyophilized equine amnionic membrane and amnionic fluid have been processed for use in equine joints but are no longer commercialized in that form. Amnionic membrane has evolved as a relatively thin, tough membrane designed to resist shear and tension due to amnionic fluid distention and pressure produced by fetal movements in utero. In contrast, umbilical tissue is designed inherently to provide structural support and cushion to protect the vessels from compression due to external pressure or torsion, a function more aligned with a need for compressive stiffness, such as in articular cartilage.

v. Umbilical Cord (UC)

Umbilical tissue contains connective tissue substances in a loose matrix network that can be both structural and supplemental to natural trophic and anti-inflammatory processes. In the literature for human umbilical cord, the term Wharton's Jelly (WJ) is coined for this umbilical tissue that is neither vessel nor the encasing external amnionic layer of the umbilical cord. Several studies show this connective tissue contains proteinaceous fluid, collagens and glycosaminoglycans. While further research is needed, placental tissue has been successfully used in over 180 homologous sites as tissue supplement grafts in humans, showing significant improvements in pain and function scores without reported adverse events. In an online search, serious adverse biologic response or complications to WJ were not noted. Lack of adverse events has putatively been attributed to the primitive nature of the tissue and immune privilege that has also recognized in human and equine stem cells. Intra-articular injection of allogeneic equine stem cells did not elicit a CD4+ lymphocyte reaction, as was seen after xenogeneic stem cells. This minimally manipulated, equine connective tissue was derived from equine umbilical cord and has some analogy to WJ in humans.

vi. Equine Micro-Particulate Allografts

In the equine species, micro-particulate, flowable allografts have been reported and include dental pulp and amnionic membrane sources. Dental pulp tissue harvested from unerupted neonatal tooth buds of deceased foals and processed by physical means to microparticles demonstrated efficacy in a controlled, prospective clinical trial for the treatment of osteoarthritis and desmitis in lame horses. The product is no longer available. Several amnionic products have been commercialized, in some cases with little characterization or evidence of its safety. Currently marketed amnionic tissue allografts may differ in formulation and/or route of administration from published work or safety reports.

The results described herein show that the umbilical cord is a unique, and potentially preferable connective tissue resource due to its high fetal protein content, the immune privilege attributed to tissues of fetal origin and connective tissues inherent purpose to supplement and protect the structure of surrounding tissues.

Bio scaffolds from connective tissue can provide structural supportive networks to damaged tissue and allow for an improved tissue microenvironment. These extracellular connective tissue matrices can be applied to fill tissue voids in damaged or torn tissue and potentially serve as a structural foundation and supplement for the endogenous cells to adhere, migrate into, and begin the process of healing and repair. Additionally, bioscaffolds placed locally can re-stimulate the process of healing and repair by rejuvenating the microenvironment with fetal proteins that can reinvigorate dormant endogenous cells in poorly healed scarred tissue. Advantages of this particulate, equine fetal tissue allograft for structural supplementation of MSK tissues include a biodegradable, natural source designed to resist tissue compression and torsion, and one that is amenable to injectable delivery and convenient to use. The underlying quality of the product is assured by cryopreservation of fresh tissue, donor health and uniformity, and a meticulously selected tissue source of low immunogenicity.

This Example characterizes the protein content of cryopreserved, micro-particulate equine umbilical cord-derived (UC) allograft suspension and to evaluate its in vivo biocompatibility for potential supplementation into equine joint, tendon and ligament tissue.

### 2. Methods

i. Characterization

Cryopreserved (−80 °C) a fillet-derived equine umbilical tissue suspension from eleven (11) different approved single-sourced donors that had met inclusion criteria, plus two additional samples from one donor (13 total samples), were thawed and processed by mass spectrometry (MS), tandem mass spectrometry (MS/MS), and Enzyme-Linked Immunosorbent Assay (ELISA). Proteomic analysis included protein identification, relative quantification using total spectral counts (TSC), and function. Using high resolution MS and MS/MS, proteins and post translational modifications were identified using a procedure that included tissue processing and mass analysis on Orbitrap Fusion Tribrid mass spectrometerd coupled to an nEasy-LC1000 nano liquid chromatography. Tandem mass spectra (MS/MS) were searched against the Equus caballus proteome set (.uniprot.org/proteomes/UP000002281) using Thermo Proteome Discoverer 2.5.0.400 and UniProt public databases. Identified proteins (name and accession number) were sorted for protein expression from most abundant to least abundant using a semiquantitative analysis of protein content expressed as total spectral count (TSC). Spectral counts over 100 were designated as high based on all the TSC counts from all the other proteins. Protein TSCs detected in all 11 donors were analyzed further for variability within and among donors and searched for function. Spectral counts over 40 were considered relevantly abundant. Using multi-plex ELISA, fillet-derived equine umbilical tissue suspensions were analyzed for concentration of interferon gamma (IFNg), interleukin-1 alpha (Il-1alpha), interleukin-1 receptor antagonist (Il-1ra), interleukin-2 (Il-2), interleukin-4 (Il-4), interleukin-8 (Il-8), interleukin10 (Il-10), interleukin-15 (Il-15), monocyte chemoattractant protein-1 (MCP-1), vascular endothelial growth factor (VEGF), and hyaluronic acid.

ii. In Vivo Studies
a. Controlled Study in Clinically Normal Horses:

This was a two-dose (Day 0 and Day 21), masked, randomized, crossover design study with clinical and analytical evaluations of several parameters related to clinical and tissue biocompatibility performed in accordance with prior approval by the research facility's IACUC (ETCR-24-03). Candidate horses underwent a 7-day acclimation period and met the following inclusion criteria: Light saddle breeds, >3 years of age, normal health, hematologic and clinical chemistry parameters, including SAA, no apparent lameness and clinically normal carpi and suspensory ligaments based on normal appearance, palpation, range of motion for joints and pain on pressure for ligaments. Baseline evaluations were documented before initial injections on Day 0.

Concurrently, four horses started the study with the joint administration and four horses started the study with the ligament administration. Each horse had a single dose of fillet-derived equine umbilical tissue suspension (1.5 mL; as <100 micron-sized particles at a concentration 50 mg of per Milliliter) administered into one randomly assigned midcarpal joint or randomly assigned lateral branch of the front suspensory ligament. Twenty-one days later, the alternate joint or ligament was injected with fillet-derived equine umbilical tissue suspension resulting in a 42-day study with each horse receiving both a joint and ligament injection. The contralateral joint and ligament were injected contemporaneously with an equivalent volume of sterile saline (0.9% Sodium Chloride) control solution (C). In a blinded fashion, serial observations, physical and lameness examinations, synovial fluid analyses, pain on flexion or pressure scores, swelling circumference measurements and clinical pathology analyses were evaluated for effects of fillet-derived equine umbilical tissue suspension compared to baseline, controls and administration of two doses 21 days apart. Adverse events were recorded.

Physical examinations, pain, and inflammation evaluations were conducted once on Day 0 (before) and on Days 1, 3, 5, 10, and 21 after injections by a blinded clinician to evaluate rectal temperature (T), pulse rate (P), and respiratory rate (R), SSA, synovial fluid analysis for joints, scoring for pain on flexion (joints; 0-4), pain on pressure (ligaments; 0-4), and swelling (circumference [cm]). Scores were 0=none; 1=minimal; 2=mild; 3=moderate; and 4=marked. Lameness evaluations (clinical and kinematic) were conducted on Days 0, 3, 5, 10, and 21 post-injections using the lameness classification system as the primary lameness outcome. Kinematic video recordings provided a video record of the horse for use as a reference if needed. Blood samples for hematology and chemistry panels (Day 0 and 21) and serial synovial fluid samples were analyzed for cell counts, differentials and total protein. Synovial fluid was further analyzed quantitatively for albumin and globulin. Synovial fluid was also analyzed by ELISA for the same cytokines as above.

b. Case Study in MSK Diseased Horses:

This was a single-dose prospective case series with baseline control and post-treatment clinical and analytical evaluations of several factors related to biocompatibility in three research horses with lameness due to osteoarthritis, desmitis, and tendonitis. The study was conducted in accordance with an approved animal use protocol by the research facility's IACUC (ETCR-24-03). Horses were acclimated to study conditions of stall confinement for one month and enrolled four days prior to Day 0 when baseline control data were collected. Horses were healthy, as determined by daily general health observations and physical examinations. Baseline digital radiographic imaging (four standard views) was conducted on two horses confirming joint abnormalities of the carpus (one radiocarpal and one mid-carpal joint) including enthesiophytes, osteophytes, irregular joint surfaces and evidence of old bone injury. Baseline digital ultrasound imaging (cross-sectional and longitudinal sections of the flexor tendons and suspensory ligament from the carpus to the fetlock) was conducted on one horse confirming desmitis and tendonitis of the accessory ligament of the deep digital flexor tendon and superficial digital flexor tendon. Similar imaging was performed at the end of the study. Serial outcomes for lameness, pain, enlargement, and inflammation (local and systemic) were completed in all horses using the same methods as in the first in vivo study on clinically normal horses.

Two horses with lesions of carpal OA received a single, intra-articular injection of 1.5 mL of fillet-derived equine umbilical tissue suspension into the affected joint on Day 0. A single horse with forelimb desmitis and tendonitis received a single intra-lesional injection of fillet-derived equine umbilical tissue suspension on Day 0; 1.5 mL was injected directly into the tendon and ligament lesion. Physical examinations, pain, and inflammation evaluations were conducted once on Days 0, 1, 3, 5, 10, 14, 21, and 28 to evaluate various clinical biocompatibility parameters. Lameness evaluations (clinical and kinematic) were conducted on Days 0, 3, 5, 10, 14, 21 and 28 post-injections. Blood samples for hematology and chemistry panels were collected on Days 0 and 28. Blood samples for SAA measurement and synovial fluid for clinical pathology and cytokine analyses were collected on Days 0, 1, 3, 10, and 28. The trial terminated on Day 28.

c. Controlled Pathologic Study for Acute Tissue Biocompatibility:

This was a controlled, cross-over design multiple-dose study of the clinical and pathological response of joint tissues (carpus and fetlock), ligament (lateral branch of the suspensory ligament), and pectoral lymph nodes to a similar dose 1 (1.5 mL) of fillet-derived equine umbilical tissue suspension administered by injection. Assigned joints and ligaments were normal on radiography and ultrasound prior to the start of the study. Ultrasound guided injections confirmed the placement of the fillet-derived equine umbilical tissue suspension within the center of the ligament along a ~2 cm length. Contralateral joints and ligaments were similarly collected and processed as controls (C). Each of two horses had two joints in one randomly selected limb (carpus and fetlock, alternated in each horse) injected with fillet-derived equine umbilical tissue suspension 2 days or 5 days prior to, and ligament 2 days prior to, euthanasia for reasons unrelated to MSK or general health abnormalities. Horses received a non-steroidal anti-inflammatory drug orally (2 gm BID) for 3 days, physical examinations daily, and comfort assessed twice daily in the 3×3m stall. Synovial fluid gross appearance and total protein were performed at injection and tissue harvest. Immediately after euthanasia, synovial membrane, joint capsule, ligament, articular cartilage, and pectoral lymph nodes were harvested by dissection at multiple sites, both near and away from the injection site. Specimens from fillet-derived equine umbilical tissue and C tissues were fixed in formalin, embedded in paraffin and sectioned (5 um). All tissue sections (fillet-derived equine umbilical tissue suspension and C) were stained with hematoxylin and eosin (H&E). Articular cartilage was also stained with metachromatic toluidine blue to assess the quantity, location, and pericellular content of aggrecan. The surface of articular cartilage was evaluated for the presence of fillet-derived equine umbilical tissue. Histologic sections of fillet-derived equine umbilical tissue suspension and C tissues and corresponding lymph node were assessed in a blinded fashion by a board-certified veterinary pathologist (DR) for inflammation, tissue architecture, and the potential presence of allograft.

d. Statistical Analyses:

Descriptive statistics were performed within and among donors and included mean +/−SD and CV (%) and cut-offs for TSC abundance were used to classify proteins relative quantity. Objective clinical and synovial fluid data were further analyzed by paired t-test and two-factor analysis of variance (fillet-derived equine umbilical tissue suspension vs C and Day). Ordinal scored data were reported as median and range. Significance was set at P<0.05. Outcome data from ELISA were graphed (mean +/−SD and SEM) and outcomes with statistical significance were additionally graphed as % change from baseline to show trends over time using each horse as its own control.

### 3. Results:

i. Proteomics:

Two thousand six hundred and forty-five (2645) TSCs were identified and annotated with a protein name and unique accession number. Rare, annotated proteins with TSC of 3 or less represented eleven hundred and eighty-two (1182) TSCs, contributed insignificantly to the total protein content, were present in only 3% of donors, therefore, were not further analyzed.

TABLE 1
Selected relevant proteins and functions in moderate abundance¹

| Protein Name | Function Summary |
|---|---|
| Chondroitin sulfate | Key ECM component of cartilage aggrecan and all |
| proteoglycan | connective tissues: PG and GAGs; Key role: Protection |
| | from compression |
| Biglycan Proteoglycan | Found in bone, cartilage, tendon; contains GAG chains; |
| in ECM | interacts with decorin and other small PGs. Protection for |
| | compression and ECM integrity |
| Asporin | Regulator of cartilage formation; development of tendons |
| | and ligaments; collagen mineralization for endochondral |
| | ossification, polymorphisms of protein associated with |
| | osteoarthritis |
| Versican Core Protein | Key component of extracellular matrix (ECM), regulates |
| | cell growth, migration and adhesion |
| Collagen type 1 alpha | Structural component of all connective tissues, particularly |
| | bone, ligament, tendon. Affects joint laxity |
| Four smaller collagens | Structural support and binding to collagen type 1 to |
| in ECM | stabilize the ECM |
| Superoxide dismutase | Protective; breaks down harmful oxygen molecules such as |
| | free radicals; antioxidant |
| Annexin A1 | Anti-inflammatory, tissue repair, regulates cell proliferation |
| Heat Shock Protein 90 | Suppresses stress, protein folding, cell signaling, immunity |
| Galectin | Regulator of immune and inflammatory responses |
| Tenascin | ECM glycoprotein - protection from compression |
| Integrin Subunit | ECM; transmit mechanical signals to cell, critical role in |
| alpha (3) | ECM of interstitial tissues |
| Calpain | plays a pivotal role in MMP 2 activation and extracellular |
| | matrix remodeling, neurogenesis |
| Proline and arginine | Functions in stem cell differentiation to ligament, |
| rich end leucine rich | downregulates complement on damaged cartilage and |
| repeat protein | inflammation in rheumatoid arthritis |

¹Moderate Abundance = TSC of 40-99

Consistency in protein content between different donors was evident even in lower abundance (mean 4-19) TSCs. As mean TSC increased, so did the percent of donors that expressed that protein. Specifically, at a mean TSC of 4, nearly one-half of the proteins (44%) were expressed in all donors, a large increase from 3% expressed at mean TSC of 3 or less. At a mean TSC of 5, 6 and 7, 74%, 81%, and 95% of every donor expressed the protein, respectively. Of the 506 proteins in the TSC range of 8-17, 96%-100% were expressed in every donor.

Proteins with a mean TSC of 18 to 39 were considered of lower relative abundance, however, were present in every donor. Even at lower abundance these proteins may have a highly relevant function. Therefore, proteins with mean TSC of 18-39 were further analyzed and proteins with <20% CV identified with consistent presence within the fillet-derived equine umbilical tissue. Some proteins in this group include Insulin-like growth factor 2 (anabolic particularly to chondrocytes), Clusterin (protection from oxidative injury), dimethylarginine dimethylaminohydrolase 2 (Nitric Oxide Synthase inhibitor, anti-inflammatory), cadherin (mediates cell-cell adhesion), many fibrillar collagens and matrix glycoproteins, to name a few. (Full Proteomics Analysis Data may be available upon request).

Proteins with a mean TSC of 40-99 were considered of moderate relative abundance and were present in this abundance range in every donor. These proteins have significant biologic roles in tissue structure (fibrillar such as collagens, and hydroscopic such as glycans), tissue repair, metabolism, protective to tissue and cellular homeostasis (antioxidant), energy generation and homeostasis etc. Select and highly relevant, notable proteins are represented within this range.

### (Table 1)

Proteins with a range mean TSC of 100 or greater [80 proteins] were considered as highly abundant and were present in high abundance in all donors with few exceptions and all proteins of all donors had high-moderate abundance with a median TSC of 89 or greater. The predominant functions of these well-defined proteins included structural support and protection of connective tissue through a variety of roles and regulatory functions in connective tissue matrix.

TABLE 2
Selected relevant proteins and functions in high abundance¹

| Protein Name | Function Summary |
|---|---|
| Alpha 2 macroglobulin | Inhibits proteases, clears misfolded proteins, inhibits |
| | complement, binds growth factors, controls inflammation |
| | (binds IL-6, IL-1, TNF). Considered key active ingredient in |
| | plasma products. |
| Heparan sulfate | Proteoglycan in cartilage and tissues; acts as receptor, |
| proteoglycan 2 | profound effects on growth factor action, cell adhesion and |
| | tissue architecture; turgidity |
| Annexin (lipocortin) | functions as a lipocortin (adipose corticoid); suppresses |
| | inflammation |
| Decorin | Key interstitial (ECM) Small Proteoglycan, Adhesion of |
| | Cartilage Aggrecan to Type II Collagen, fibrillogenic |
| Transgelin | TGF-β Inducible, ∴ active in growth and development; stem |
| | cell differentiation |
| Vinculin | Cell-matrix adhesion, interacts with integrin, cadherin for |
| | matrix integrity and turgidity |
| Collagens VI, XII, IV | Filaments, Collagen 1 interaction with matrix, fibrillogenic |
| Thrombospondin | ECM protein, cellular attachment and migration |
| Profilin | ECM protein, cellular attachment and migration |
| Tubulin | Structural support, transport, cell division |
| Actinin | A pleiotropic, anchoring protein |
| TGF-β induced | Binds collagen and proteoglycans, TGF tissue development, |
| protein ig-h3 | cell adhesion, tumor suppressor, cornea healing |
| Lumican | Main Keratan Sulfate Proteoglycan, assists collagen assembly, |
| | found in healing and high in Cornea |
| Plectin | Crosslinks filaments to cell membrane, stabilizes ECM and |
| | cellular tissue composite |

¹High abundance = TSC of 100+

In the triplicate samples from one donor, the protein patterns described above were mirrored in all three samples with 80 proteins that had a mean TSC of 100 or greater, the same as the mean of the 11 donors. The CV between the samples for all 245 proteins with a mean TSC of 100 or greater, ranged from 0.6-13.3%, with a mean of 4.7%, falling within the strictest standards of variability.

ii. ELISA:

Cytokine concentrations in fillet-derived equine umbilical tissue suspension (nonparticulate) are reported in Table 3. Values for the inflammatory cytokines (INFg, Il-1, Il-2, Il-4, Il-8, Il-15) were low, near the low end of the standard curve (INFg, Il-2, Il-4, and Il-8) or near the limit of detection (Il-15). Interkeukin-18, Il-1, and Il-1ra were <1000 pg/mL.

TABLE 3
Fillet-derived equine umbilical tissue suspension
mediator content as determined by ELISA¹

| Mediator (pg/mL) | Mean +/SEM | Coefficient of Variance within Donor (CV %) |
|---|---|---|
| IFNg² | 184.5 ± 32.1 | 30.1 |
| IL-1a³ | 969.3 ± 5.9 | 1.1 |
| IL-1ra⁴ | 317.9 ± 15.6 | 8.5 |
| IL-1ra/IL-1a | 0.3 ± 0.02 | 9.6 |
| IL-2⁵ | 839.7 ± 19.9 | 4.1 |
| IL-4⁵ | 85.5 ± 21.7 | 43.9 |
| IL-8⁵ | 870.8 ± 30.4 | 6.1 |
| IL-10⁵ | 95.1 ± 22.5 | 40.9 |
| IL-15⁵ | 689.2 ± 20.3 | 5.1 |
| MCP⁶ | 1175.8 ± 25.7 | 3.8 |
| VEGF⁷ | 833.7 ± 26.0 | 5.4 |
| HA⁸ | 42.3 ± 3.6 | 14.9 |

¹N = 3; samples run in triplicate;
²IFNg = Interferon gamma;
³IL-1 = Interleukin-1a;
⁴IL-1ra = Interleukin-1 receptor antagonist;
⁵IL-2, -4, -8, -10, and -15 = the corresponding interleukin;
⁶MCP-1 = Monocyte Chemoattractant Protein-1;
⁷VEGF = Vascular Endothelial Growth Factor;
⁸HA = Hyaluronic Acid

iii. Controlled Study in Clinically Normal Horses:

All 8 horses of various breeds completed the study, 5 male castrates and 3 females, ranging in age from 8 to 20 years and ranging in body weight from 750 to 1150 pounds. All hematology and chemistry blood parameters were within acceptable limits for the duration of the study. Low SAA values were sustained for the duration of the study with a mean baseline value of 32. SSA values after injection decreased significantly to a mean of 6 for the duration of the study.

### (Table)

TABLE 4
Mean ± SEM Serum Amyloid A for fillet-derived equine
umbilical tissue suspension (UC) or control (C) for 8 Horses

| | Duration After Injection (Days) | | | | | |
|---|---|---|---|---|---|---|
| Outcome | 0 | 1 | 3 | 5 | 10 | 21 |
| Serum Amyloid A | 32 ± 14 | 8 ± 6 | 8 ± 2 | 4 ± 1 | 39 ± 38 | 24 ± 19 |
| (SAA)⁶ | | | | | | |
| 1^{st} injection | | | | | | |
| Serum Amyloid A | 7 ± 5 | 6 ± 3 | 8 ± 3 | 4 ± 2 | 6 ± 2 | 6 ± 4 |
| (SAA)⁶ | | | | | | |
| 2^{nd} injection | | | | | | |

⁶SSA values of 100 were considered within normal range for the general horse population, with 300-450 indicating systemic inflammation, and over 500 indicating possible infection.

Key outcome parameters for carpal joint and ligament after fillet-derived equine umbilical tissue suspension and C injection are listed in Tables 5 and 6, respectively. Horses administered fillet-derived equine umbilical tissue suspension or C in either the joint or ligament did not demonstrate clinical signs of lameness, pain or inflammation after Day 1 for at least 21 days or after the second injection for a total of 42 days. Scores for pain on pressure/flexion and lameness, as well as values for circumference (swelling), were not different between C or fillet-derived equine umbilical tissue suspension or from baseline. Joint fluid analyses demonstrated a neutrophilic transudative synovitis that peaked on Day 1 which was greater in fillet-derived equine umbilical tissue suspension than in C and had returned to baseline and normal values by Day 10. (Table 5) Horses administered C or fillet-derived equine umbilical tissue suspension at a different site 21 days after the first injection also did not have clinical signs of pain, lameness or inflammation for at least 21 days and showed a similar neutrophilic transudative synovitis that peaked on Day 1 that had returned to baseline and normal values by Day 10. No adverse events attributable to the C or fillet-derived equine umbilical tissue suspension administration were observed.

TABLE 5
Mean ± SEM or Median [range] Joint Outcomes for fillet-derived equine umbilical tissue suspension (UC) or control (C) for 8 Horses/site

| | | Duration After Injection (Days) | | | | | |
|---|---|---|---|---|---|---|---|
| Outcome | Joint | 0 | 1 | 3 | 5 | 10 | 21 |
| Circumference (cm) | C | 29.2 ± 0.5 | 29.8 ± 0.4 | 26.9 ± 1.1 | 27.2 ± 0.8 | 28.5 ± 0.8 | 29.6 ± 0.2 |
| | UC | 29.9 ± 0.4 | 30.1 ± 0.3 | 28.2 ± 1.1 | 27.5 ± 0.8 | 28.6 ± 0.7 | 29.4 ± 0.4 |
| Pain of | C | 0 | 0 | 0 | 0 | 0 | 0 |
| Flexion (0-4)¹ | UC | 0 | 0 | 0 | 0 | 0 | 0 |
| Lameness | C | 0 | 0 | 0 | 0 | 0 | 0 |
| Score (0-4)² | UC | 0 | 0 | 0 | 0 | 0 | 0 |
| Synovial | C | 137.5 ± 18.3 | 1850^{a} ± 594.3 | 714.3^{a} ± 281.5 | 1,450^{a} ± 979.1 | 312.5 ± 100.8 | 187.5 ± 39.8 |
| Fluid TNCC³ | UC | 171.4 ± 28.6 | 21,237.5^{a, b} ± 5,980.7 | 712.5^{a} ± 344.8 | 1,237.5^{a} ± 244.2 | 328.6 ± 74.7 | 212.5 ± 47.9 |
| Synovial | C | 1.2 ± 0.1 | 1.7^{a} ± 0.4 | 1.4 ± 0.2 | 1.8 ± 0.2 | 1.3 ± 0.1 | 1.3 ± 0.1 |
| Fluid Total | UC | 1.2 ± 0.1 | 3.3^{a, b} ± 0.4 | 1.4 ± 0.2 | 1.8 ± 0.1 | 1.5 ± 0.1 | 1.3 ± 0.1 |
| Protein⁴ | | | | | | | |
| Synovial | C | 0.7 ± 0.1 | 1.0^{a} ± 0.2 | 0.8 ± 0.1 | 1.0 ± 0.1 | 0.8 ± 0 | 0.9 ± 0 |
| Fluid | UC | 0.7 ± 0.1 | 1.6^{a, b} ± 0.2 | 0.8 ± 0.1 | 1.1 ± 0 | 0.9 ± 0 | 0.9 ± 0.1 |
| Albumin⁵ | | | | | | | |
| Synovial | C | 0.5 ± 0 | 1.1^{a} ± 0.4 | 0.6 ± 0.1 | 0.8 ± 0.2 | 0.6 ± 0.1 | 0.5 ± 0.1 |
| Fluid | UC | 0.6 ± 0.1 | 1.3^{a} ± 0.3 | 0.7 ± 0.1 | 0.8 ± 0.1 | 0.6 ± 0.1 | 0.6 ± 0.1 |
| Globulin⁵ | | | | | | | |
| Synovial | C | 9.0 ± 7.2 | 52.6^{a} ± 13.9 | 16.0^{a} ± 9.6 | 16.1^{a} ± 5.8 | 9.3 ± 4.3 | 2.8 ± 0.9 |
| Fluid | UC | 4.3 ± 2.6 | 65.4^{a, b} ± 6.3 | 18.6^{a} ± 7.8 | 11.6^{a} ± 6.8 | 3.7 ± 0.9 | 5.8 ± 1.4 |
| PMN (%)⁶ | | | | | | | |

¹Pain Scores: 0 = none, 1 = minimal, 2 = mild, 3 = moderate, 4 = marked;
²Lameness Classification: 0 = no lameness; 1 = intermittent lameness at trot; 2 = consistent lameness under special circumstances; 3 = Consistent lameness at the trot; 4 = Lameness at the walk; 5 = Non-weightbearing lameness
³TNCC = Total Nucleated Cell Count (Cells/uL); Normal range is <1000 TNCC/μL
⁴Total Protein values <2.0 g/dL are within acceptable limits, however, typically are <1.5 g/dL in synovial fluid. Values ranging from 2.0-4.5 gm/dL reflect a transudate and over 4.5 g/dL reflect an exudate.
⁵Normal synovial fluid values for albumin are <1.2 g/dL and for globulin are <3.3 g/dL.
⁶Normal PMN percentage is <10%
^{a}Significantly differs from baseline (P < 0.05)
^{b}Significantly differs between C and fillet-derived equine umbilical tissue suspension (P < 0.05)
—Synovial Fluid Days 0,1,3 for the first four horses was not analyzed at the laboratory due to an error

TABLE 6
Mean ± SEM or Median [range] Ligament Outcomes for fillet-derived equine
umbilical tissue suspension (UC) or control (C) for 8 Horses/site

| | | Duration After Injection (Days) | | | | | |
|---|---|---|---|---|---|---|---|
| Outcome | Ligament | 0 | 1 | 3 | 5 | 10 | 21 |
| Circumference (cm) | C | 21.4 ± 0.4 | 21.6 ± 0.3 | 21.6 ± 0.6 | 21.9 ± 0.6 | 22.1 ± 0.8 | 21.6 ± 0.7 |
| | UC | 21.3 ± 0.3 | 21.5 ± 0.3 | 21.7 ± 0.4 | 22.0 ± 0.6 | 21.7 ± 0.6 | 21.5 ± 0.6 |
| Pain on | C | 0 | 0 | 0 | 0 | 0 | 0 |
| Pressure (0-4)¹ | UC | 0 | 0 | 0 | 0 | 0 | 0 |
| Lameness | C | 0 | 0 | 0 | 0 | 0 | 0 |
| Score (0-4)² | UC | 0 | 0 | 0 | 0 | 0 | 0 |

¹Pain Scores: 0 = none, 1 = minimal, 2 = mild, 3 = moderate, 4 = marked
²American Association of Equine Practitioners Lameness Grades: 0 = no lameness; 1 = intermittent lameness at trot; 2 = consistent lameness under special circumstances; 3 = Consistent lameness at the trot; 4 = Lameness at the walk; 5 = Non-weightbearing lameness

Synovial fluid total protein (g/dL) analysis was completed on four horses for Days 0, 1 and 3 and all 8 horses for other days. Total protein synovial fluid values were within acceptable range (≤2.0 g/dL) and near normal typical range (≤1.5 g/dL) at most time points with the greatest increase at Day 1 in both C and fillet-derived equine umbilical tissue suspension (i.e. a transudate). Synovial fluid albumin (g/dL) increased on Day 1, was greater in fillet-derived equine umbilical tissue suspension than C, above the normal range, and quickly returned to and sustained normal values on Day 3. Synovial fluid globulin (g/dL) did not increase above the normal range but was greater than baseline in both C and UC. Horses administered C or fillet-derived equine umbilical tissue suspension at a different site 21 days after the first injection did not differ in synovial fluid protein values. (Table 5).

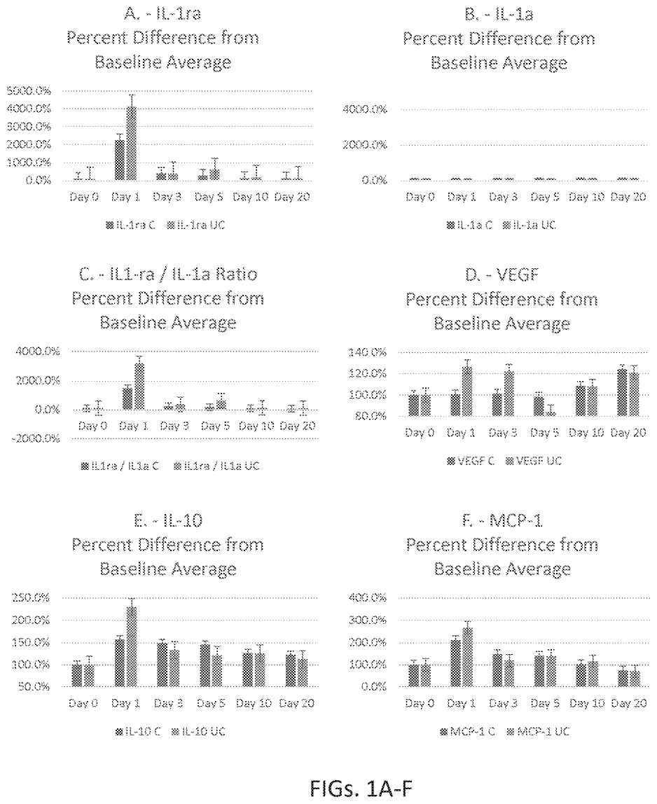

Synovial fluid mediator concentrations were low and did not significantly change for the inflammatory cytokines Il-2, Il-4, Il-8, and Il-15 (Table 7). Il-1ra however increased significantly (P<0.001) on Days 1, 3 and 10 after fillet-derived equine umbilical tissue suspension injection and were greater than saline control and baseline. (FIG. 1) In all horses, synovial fluid Il-1ra was increased over the paired baseline value for the duration of the study (7 of 8 horses) or until Day 10 (1 horse) after injection with fillet-derived equine umbilical tissue suspension that did not occur similarly after injection of saline. The statistically significant increase of Il-1ra with fillet-derived equine umbilical tissue suspension administration resulted in an increase of the Il-1ra to Il-1 ratio (Il-1ra/Il-1) several-fold greater than baseline and C. (P<0.001) This statistically significant increase was also seen at Day 5 indicating a fillet-derived equine umbilical tissue suspension effect that persisted longer than the transient neutrophilic response. The ratio remained >1.0 for all horses to Day 10 or longer (FIG. 1; Table 7). Il-10 was significantly greater (P<0.01) than both baseline and C after fillet-derived equine umbilical tissue suspension injection. (FIG. 1) Monocyte Chemoattractant Protein (MCP)-1 significantly increased after UC and C injection compared to baseline on Day 1 and fillet-derived equine umbilical tissue suspension was greater than C (P<0.05) on Day 1. (FIG. 1) MCP-1 values returned to baseline values without any other increase in the subsequent 20 days of the study.

TABLE 7
Mean ± SEM Cytokine concentration (pg/mL) in synovial fluid

| | | Duration After Injection (Days) | | | | | |
|---|---|---|---|---|---|---|---|
| Cytokine | Joint | 0 | 1 | 3 | 5 | 10 | 21 |
| IFNg | C | 426.6 ± 245.1 | 607 ± 421.6 | 661 ± 455.2 | 548.6 ± 308.3 | 600 ± 397.9 | 507.1 ± 343.2 |
| | UC | 207.1 ± 41.4 | 515.4 ± 305.6 | 349.3 ± 133.7 | 292.8 ± 102.8 | 313.5 ± 92.1 | 307.8 ± 57.2 |
| IL-1a | C | 947 ± 78.7 | 1015.5 ± 106.9 | 1119.9 ± 103.5 | 1168.3 ± 201.2 | 1160.6 ± 226.9 | 1266.9 ± 170.8 |
| | UC | 973.7 ± 118.9 | 969.7 ± 130.2 | 1114.9 ± 172.4 | 969.9 ± 78.6 | 1106.5 ± 185.8 | 1086.5 ± 158.8 |
| IL-1ra | C | 657.6 ± 225.1 | 8785.7 ± 2567.3 | 1767.6 ± 332.9 | 1294.1 ± 293.9 | 829.8 ± 322.2 | 687.9 ± 305.2 |
| | UC | 437.8 ± 72.4 | 15635.2 ± 5565.6 | 1625.6 ± 332.3 | 2418.1 ± 1260.6 | 729.9 ± 141.9 | 525.6 ± 91.9 |
| IL-1ra/ | C | 0.7 ± 0.2 | 10.1 ± 3.6 | 1.6 ± 0.2 | 1.2 ± 0.3 | 0.6 ± 0.1 | 0.5 ± 0.1 |
| IL-1a | UC | 0.5 ± 0.1 | 16.4 ± 4.3 | 1.9 ± 0.5 | 3.2 ± 2.0 | 0.7 ± 0.1 | 0.5 ± 0.1 |
| IL-2 | C | 1089.1 ± 116.3 | 1218.3 ± 201.3 | 1483.4 ± 254.3 | 1711.5 ± 337.5 | 1500.9 ± 282.4 | 1270.5 ± 202.5 |
| | UC | 1072.9 ± 113.2 | 1125.8 ± 160.1 | 1317.6 ± 148.6 | 1295.6 ± 122.6 | 1501.6 ± 349.3 | 1250.5 ± 166.1 |
| IL-4 | C | 272.4 ± 160.3 | 401.8 ± 290.0 | 477.3 ± 351.5 | 347.66 ± 199.7 | 309.4 ± 198.6 | 343.0 ± 247.0 |
| | UC | 155.5 ± 47.9 | 339.1 ± 203.6 | 253.5 ± 133.7 | 191.9 ± 72.0 | 213.6 ± 75.1 | 203.5 ± 63.6 |
| IL-8 | C | 867.5 ± 63.5 | 1099.3 ± 98.5 | 1046.9 ± 81.5 | 1483.8 ± 522.4 | 1484.8 ± 455.5 | 1150.4 ± 145.0 |
| | UC | 872 ± 128.9 | 1169.8 ± 245.1 | 1439.1 ± 435.9 | 1131.3 ± 299.0 | 2934.6 ± 2061.1 | 1056.4 ± 162.8 |
| IL-10 | C | 526.8 ± 166.8 | 824.7 ± 258.4 | 784.2 ± 334.8 | 764.8 ± 277.7 | 664.1 ± 297.8 | 643.8 ± 274.9 |
| | UC | 411.3 ± 51.9 | 943.7 ± 266.0 | 546.3 ± 117.6 | 498.6 ± 83.3 | 515.0 ± 84.3 | 461.5 ± 61.4 |
| IL-15 | C | 823.5 ± 104.1 | 959.8 ± 207.2 | 1169.8 ± 220.0 | 1294.4 ± 218.6 | 1192.4 ± 265.0 | 1049.6 ± 203.4 |
| | UC | 759.8 ± 97.8 | 949.4 ± 194.0 | 1028.0 ± 142.1 | 1043.3 ± 107.9 | 1185.3 ± 238.6 | 938.3 ± 103.4 |
| MCP-1) | C | 4874.9 ± 2119.5 | 10265.3 ± 4536.8 | 7212.0 ± 3223.2 | 6810.6 ± 2438.1 | 4967.0 ± 1278.1 | 3629.6 ± 598.8 |
| | UC | 5479.0 ± 2259.0 | 14610.8 ± 4737.8 | 6471.8 ± 1933.9 | 7635.8 ± 2405.7 | 6312.1 ± 3016.4 | 3896.0 ± 1009.2 |
| VEGF | C | 1608.5 ± 222.5 | 1617.9 ± 105.9 | 1630.2 ± 133.7 | 1583.4 ± 136.6 | 1747.3 ± 200.8 | 2001.3 ± 158.1 |
| | UC | 1523.6 ± 259.9 | 1929.2 ± 252.7 | 1860.9 ± 197.3 | 1278.1 ± 116.3 | 1642.4 ± 281.5 | 1842.0 ± 238.0 |

C = Control;
UC = fillet-derived equine umbilical tissue suspension

Case Study in MSK Diseased Horses: Three horses completed the study and ranged in age from 12 to 18 years, two females and one male castrate. No adverse events attributable to the fillet-derived equine umbilical tissue suspension administration were observed. All horses showed at least one grade of improvement in lameness and/or pain compared to baseline values. (Table 8)

TABLE 8
Outcome Scores for Lameness and Pain for Horses
with Osteoarthritis or Desmitis and Tendonitis

| | Evaluation Day | | | | | | | |
|---|---|---|---|---|---|---|---|---|
| Score | 0 | 1 | 3 | 5 | 10 | 14 | 21 | 28 |
| Mean Lameness Score¹ | 3 | * | 2 | 2 | 1 | 1 | 2 | 2 |
| Ligament Pressure Score² | 3 | 2 | 0 | 1 | 1 | 2 | 0 | 0 |
| Mean Carpal Flexion Score² | 1 | 1 | 0 | 1 | 0 | 0 | 0 | 0 |

* Horses not trotted for lameness on Day 1.
¹Lameness Classification: 0 = no lameness; 1 = intermittent lameness at trot; 2 = consistent lameness under special circumstances; 3 = Consistent lameness at the trot; 4 = Lameness at the walk; 5 = Non-weightbearing lameness
²Pain Scores: 0 = none, 1 = minimal, 2 = mild, 3 = moderate, 4 = marked

None of the horses developed clinical signs of increased pain, lameness or swelling after injection at any time point for the 28 days of the study. Values for circumference (swelling) were variable and did not change consistently in any horse. Joint fluid analyses demonstrated a mild to moderate transudative synovitis that returned to within normal baseline values after Day 3. (Table 9) Little alteration occurred in synovial fluid albumin and globulin and values remained below reported normal for joint fluid (1.2 and <3.3 g/dL, respectively). Synovial fluid inflammatory cytokine Il-1 values were greater at baseline in OA horses than the normal horses (Table 4) such that the starting average Il-1ra/Il-1 ratio was 0.3 (fractional and undesirable), and lower than the approximately even ratio (0.95) in the normal horses. After injection of fillet-derived equine umbilical tissue suspension in these OA joints synovial fluid Il-1ra spiked as in the normal horses, such that the Il-1ra/Il-1 ratio was converted to >4-fold and was greater than C. (Table 9). Similarly, anti-inflammatory mediators Il-10, HA, and VEGF were increased and greater than baseline and controls. Serum Amyloid A did not detect systemic inflammation caused by fillet-derived equine umbilical tissue suspension in horses with OA or desmitis and tendonitis. Digital radiography for carpi and digital ultrasound of the affected forelimb flexor tendons and suspensory ligaments were compared before treatment to study termination (Day 28) without changes noted over the course of the study. Hematology and serum chemistry were within acceptable limits at baseline and at the end of the study.

TABLE 9
Trends in Mean Synovial Fluid Values in Osteoarthritic Joints Injected
with fillet-derived equine umbilical tissue suspension.

| | Day 0 | Day 1 | Day 3 | Day 10 | Day 28 |
|---|---|---|---|---|---|
| TNCC¹ | 150.0 | 9,000 | 2,400 | 250.0 | 200.0 |
| (cells/uL) | | | | | |
| Albumin (g/dL) | 0.8 | 1.4 | 1.2 | 0.8 | 1.0 |
| Globulin (g/dL) | 0.4 | 1.2 | 0.7 | 0.6 | 0.4 |
| IL-1a | 1386.5 | 1204.0 | 1160.0 | 1271.0 | 1314.5 |
| IL-1ra | 429.0 | 4926.0 | 2086.0 | 447.5 | 636.5 |
| IL-1ra/IL-1a | 0.3 | 4.1 | 1.8 | 0.4 | 0.5 |
| IL-10 | 280.0 | 529.5 | 493.5 | 328.0 | 353.5 |
| VEGF | 1535.5 | 1905.5 | 2253.0 | 1641.5 | 1850.5 |
| HA | 127.7 | 301.2 | 303.4 | 175.0 | 122.7 |
| IFNg | 215.0 | 209.5 | 200.5 | 205.5 | 309.0 |
| IL-2 | 1095.0 | 1131.0 | 1161.0 | 1198.0 | 1202.5 |
| IL-4 | 147.0 | 142.0 | 125.0 | 117.5 | 462.5 |
| IL-8 | 1039.0 | 2017.0 | 1105.5 | 1017.5 | 1173.0 |
| IL-15 | 882.5 | 863.5 | 766.5 | 842.5 | 1028.0 |
| MCP-1 | 2082.5 | 2514.0 | 5071.0 | 1887.5 | 1965.0 |

¹TNCC = Total Nucleated Cell Count (Cells/uL); Normal range is <1000 TNCC/μL; See abbreviations in Table 3 for cytokines.
² Total Protein values <2.0 g/dL are within acceptable limits, however, typically are <1.5 g/dL in synovial fluid. Values ranging from 2.0-4.5 gm/dL reflect a transudate and over 4.5 g/dL reflect an exudate.
³ Normal synovial fluid values for albumin are <1.2 g/dL and for globulin are <3.3 g/dL.

Controlled Pathologic Study for Acute Tissue Biocompatibility: At tissue harvest, synovial membrane appeared grossly normal with one of the four joints showing minor hemorrhage at the site of injection. Other than at the site of injection, the synovium and cartilage were grossly normal, indistinguishable from the contralateral control joints. All joints had clear, viscous, yellow synovial fluid with a refractometer protein of <2.5 gm/dL in injected joints and <2.0 gm/dL in uninjected control joints. Ligaments had no gross evidence of the injection site including on cut surfaces, both longitudinal and cross-sectional through the site of the injection. Pectoral lymph nodes were grossly normal and of similar size and appearance between the injected and the uninjected control limb. Similar to the findings in the single-dose biocompatibility study, these horses with multiple fillet-derived equine umbilical tissue doses injected over 3 days (one carpus, one fetlock, and one suspensory ligament) were normal on physical examinations and general health observations. Horses were not lame in the stall and had no visible swelling after injection.

Histologic evaluation of 90 glass slides from injected and control joints, ligaments and corresponding lymph nodes were imaged in a blinded fashion using a Motic digital scanner and sent to a veterinary pathologist (DR) for evaluation using the proprietary Motic viewing software. After unblinding, all intra-articular and extraneous materials at the injection sites were further examined and compared with control tissues for presence of the experimental material. No distinct signature of the experimental product was established in the tissues examined. This suggested the injected material was readily accepted over the acute (2-5 day) period. Except for the needle tract, the subintimal synovium of none of the injected joints had evidence of reaction to foreign material. Two focal areas of acute mild inflammation were identified, and both were associated with adjacent needle tracts. In one well defined needle tract in the ligament, eosinophilic amorphous tissue was filling the tract with mild multifocal areas of inflammation visible. There was no evidence of a strong antigenic response and no foreign material transiting the regional nodes examined. The nodes examined were minimally reactive in controls and treated alike.

### 4. Discussion

The use of biologics in the management of MSK abnormalities is currently in the early stages and growing in sports medicine, orthopedics, and wound care. Given the variety in tissue source, processing, and formulation, results are few are difficult to compare and are often inconclusive or a low level of evidence. In many cases, commercialization has proceeded with insufficient characterization of the biologic product. This study focuses on a fillet-derived equine umbilical tissue. The results serve as a step to characterize and determine in vivo safety of the first reported use of fillet-derived equine umbilical tissue in equine as well as serve as a pre-clinical study for humans and other animals.

These studies evaluated both the connective tissue matrix particulate via proteomics and the suspension diluent via ELISA, to identify proteins in the bio scaffold and to determine if inflammatory cytokines leached into the diluent during processing or storage. The proteomics data found a strikingly similar rich and robust variety of relevant proteins in the fillet-derived equine umbilical tissue from each donor, supporting a consistent, stable characterization of the allograft. Variability among the more abundant proteins from the same batch (donor) was low demonstrating the high fidelity of the processing specifically for the characterization of fillet-derived equine umbilical tissue product. ELISA data was also consistent for the presence of VEGF protein and HA in the product and confirmed that the fillet-derived equine umbilical tissue suspension was low in cytokines; at or near the limit of detection, similar to values reported in normal equine joints, or similar to the normal synovial fluid reported in Table 7 as Day 0. MCP-1 and the growth factor VEGF were higher in the suspension diluent than other mediators but less than found in normal synovial fluid on Day 0. (Table 7) Importantly, UC processing did not elicit a significant release of inflammatory mediators. These data supported that the product is not delivering inflammatory cytokines at administration. The particulate fillet-derived equine umbilical tissue proteins would be expected to be released into the joint by degradation of the protein scaffold in vivo. The proteins of potential value were found in the particulate bio scaffold as indicated by the proteomics data. (See Tables 1 and 2). In addition to structural components, many proteins necessary for the regulation and control of inflammation, protection of tissues from oxidants, as well as support for cellular homeostasis necessary in extracellular matrix turnover, are consistently present in abundance in the bio scaffold particles. The presence of these proteins supports the use of this fillet-derived equine umbilical tissue as a tissue graft to be used along with medical procedures as a supplement to aid in tissue repair. In concert, these data showed a high and consistent tissue protein content with every protein defined by functions that are relevant to the health of connective tissues supporting this fillet-derived equine umbilical tissue as a homologous tissue for transfer to connective tissues.

Contributing factors that likely promoted the uniformity of results in this study included the careful and meticulous dissection of the optimal portion of the umbilical cord to maximize the desirable tissue and remove tissue that may be traumatized or influenced by outside factors prior to processing, External variables were minimized by following aseptic technique in a closed tissue processing facility under controlled standardized conditions and processing techniques. Additionally, participating farms were high-quality, closed breeding operations with valuable mares and foals. Stability and quality of the Thoroughbred donor herd were maintained under standardized policies and procedures under veterinary supervision, including the collection of umbilical cords at foaling. Each mare was fully vaccinated on a schedule for optimal transfer of immunoglobulin to the foal, tested for pathogens, and comprehensively evaluated for health throughout the gestational and parturition periods. Only umbilical cords from healthy, vigorous foals were used for the final product.

Unique to the equine studies reported here, the source of the tissue is pure fetal umbilical cord without contamination of amnion or chorionic maternal tissues. The tissue source has been processed to optimize the product, devoid of the dense vascular vessel lining and outer amnion membrane. Importantly, these abundant proteins are fetal in origin and populated the UC during gestational development. These young abundant proteins are perceived by longevity science as a “holy grail” for rejuvenating connective tissues, and some in UC are particularly associated with repair and regeneration, such as the smaller collagens and in early development, such as TGF-β proteins. These proteins are not only structural, such as the fibrillar smaller collagens and filamentous proteins, but also the critical smaller proteoglycans and proteins that affect the quality of the extracellular matrix. These protein composites assist with extracellular matrix integrity, composition, interaction with cells, tissue permeability and fluid flow that can affect tissue strength in tension and turgidity in compression. This study confirmed that the diversity and abundance of relevant connective tissue proteins in particulate UC connective tissue matrix and further supports its use as an allograft.

Transfer of the particulate allograft also delivered tissue elements with mechanical properties. These particles are anticipated to serve as a scaffold with a strong affinity for local cell migration and adhesion, based on protein composition (fibrillar collagens). The scaffold structure likely can provide a filler for tissue tears and support vulnerable early fibroblasts in a more mature tissue than is provided by a fibrin clot. In soft connective tissues, such as synovial membrane or early granulation tissue during healing, mechanical properties are defined by permeability and kinetic (k) coefficients of fluid flux through the extracellular matrix (i.e., trans synovial fluid flow). Importantly, the small proteins, glycans, chondroitin, keratan, decorin, and proteoglycans found in abundance in the fillet-derived equine umbilical tissue (Tables 1 and 2) are integral to forming networks that retain fluid both osmotically, chemically, and ionically (hydroscopic properties), properties attributed to WJ and hyaluronic acid. Hyaluronic acid was found in fillet-derived equine umbilical tissue suspension by ELISA. These small proteins, glycans, and polysulfated glycosaminoglycans define the interstitial micro pressure and permeability (pore size) that is so important for compliance (flexibility) and turgidity, properties that resist compression and protect embedded cells. These are best described for the joint; however, devices that can measure interstitial fluid pressure, flow and content are rapidly advancing this field to better understand the impact of extracellular matrix quality on tissue integrity and healing. These properties of extracellular matrix may provide supplementation that can enhance the acceleration of healing. UC connective tissue matrix delivers these proteins, in part as a suspension, and as a microparticle, functioning as an allograft, that can be targeted to the site of interest, including the soft connective tissues in joints and early granulation tissue.

Mediator analyses of synovial fluid in the in vivo studies showed that inflammatory mediators (Il-1, Il-2, Il-4, Il-8, Il-15) did not change from baseline and fillet-derived equine umbilical tissue was not different than control. The anti-inflammatory mediators (Il-1ra and Il-10) increased several-fold in joints injected with fillet-derived equine umbilical tissue compared to baseline and control joints (P<0.01) in both the in vivo clinically normal joints and the in vivo diseased joints. This effect was substantial and produced a robust positive IL-1ra/Il-1 ratio (>4-fold) in both the clinically normal joints and OA joints. In the case study, the OA joint fluid had greater Il-1 at baseline than the clinically normal joints (P<0.05). As a result, the fractional Il-1ra/Il-1 ratio (0.3) was low at baseline and reflected a profile out of balance with Il-1ra. A single injection of fillet-derived equine umbilical tissue rapidly converted the ratio to a positive Il-1ra/Il-1 ratio for greater than 5 days that remained >1 for a longer term (21 day) balance of these receptor competing proteins and slightly greater than the clinically normal joints (Il-1ra/Il-1=0.95). Although plasma and platelet products may contain greater Il-1ra than Il-1, information on the effect on the joint environment after injection is limited with only few reports in horses. One short-term study was unable to show a difference in joint fluid Il-ra 48 hrs after platelet-rich plasma injection in lipo-polysaccharide-induced synovitis compared to no treatment. In another report, Il-1 was not measured such that so the Il-1ra/Il-1 ratio could not be assessed. In a study of naturally occurring OA, a high Il-1ra/Il-1 ratio was reported but, importantly, was unable to show a statistical difference between the control and treated joints or between the before and after values. Earlier time points comparable to the study were not reported. Regardless, the consensus of the literature is that a greater amount of IL-1ra compared to IL-1 is desirable and the presumption has been the greater the ratio, the better. The high IL-1ra to IL-1 provides the balance that gives the competitive edge to IL-1ra for binding to IL-1 sites on chondrocytes and synovial cells, potentially inhibiting the release of destructive enzymes that degrade cartilage. Conclusions on relevant concentrations of IL-1 and IL-1ra in joints in vivo are elusive and limited due to the variability of these cytokine concentrations in native OA, as well as dilution by joint fluid and the inflammation associated with the injection. Therefore, the ratio of IL-1ra to IL-1 in the joint fluid continues to be given greater credence over individual values. Plasma products are mostly water, albumin, globulin, and fibrinogen (92% or more), such that growth factor proteins like insulin-like growth factor and TGF-beta, as well as IL-1ra, are present in picogram concentrations unless the plasma is concentrated. These picogram administered doses are lower than the measured nanogram concentrations in the joint fluid after fillet-derived equine umbilical tissue administration (3-4 ng/mL) or helper-dependent adenoviral-Il-1ra gene therapy (20 ng/mL). Gene therapy offers only the IL-1ra protein unlike plasma, platelets or allografts. Fillet-derived equine umbilical tissue offers the potential advantage of vast numbers of directly relevant connective tissue proteins with the additional potential advantage of being a scaffold and of fetal in origin.

IL-1β is a potent anti-inflammatory cytokine with reported chondroprotective effects [34] and was significantly greater (P<0.01) than both baseline and C after fillet-derived equine umbilical tissue injection. The potential effect of this finding is unknown, however, the scientific evaluation of IL-1β as a therapeutic gene therapy for OA has been evaluated in horses with beneficial results. Synovial fluid IL-1β was also increased after injection of isolated amnion mesenchymal stem cells in dogs with OA and was directly correlated with improvement in joint range of motion. MCP-1 is a chemokine, a group of secreted proteins within the cytokine family, whose generic function is to induce cell migration. Both chemokines and cytokines have essential roles in maintaining immune homeostasis. The early (Day 1) significant (P<0.05) increase in MCP-1 after fillet-derived equine umbilical tissue injection compared to baseline and C, along with rapid return to baseline values without any other increase in 20 days in the study (FIG. 1) supports the lack of continued immune stimulation of UC in vivo. Synovial fluid MCP-1 was decreased in dogs with OA after injection of isolated amnion derived mesenchymal stem cells and inversely correlated to an increased joint range of motion. MCP-1 has been proposed as a biomarker of joint inflammation and the results also showed an increase on Day 1 during the peak of joint inflammation.

The anabolic mediator, Vascular Endothelial Growth Factor (VEGF), was significantly increased several fold after injection with UC, compared to control and baseline (P<0.05). VEGF belongs to a sub-family of platelet-derived growth factors, specifically the cystine-knot growth factors. These are important signaling proteins involved in both vasculogenesis (de novo vessel formation) and angiogenesis (the growth of blood vessels from pre-existing vasculature). The angiogenesis and granulation tissue stage of tendon and ligament healing may benefit from increased VEGF. VEGF has an important role in the early healing of these tissues and may serve to supplement and accelerate tendon and ligament repair. In concert, these data report low synovial fluid inflammatory cytokines, except IL-1 in OA joints, and significantly increased anti-inflammatory (IL-1ra, Il-10) and growth factor mediators (VEGF) with a conversion from a fractional to a positive integer >4 for the IL-1ra/Il-1 ratio in normal and OA joints.

This example shows the safety and clinical biocompatibility of a fillet-derived equine umbilical tissue administered into clinically normal joints and ligaments in a prospective, controlled, randomized in vivo study. Biocompatibility was monitored for up to 42 days in normal horses and for 28 days in lame horses with OA and in lesions of desmitis and tendonitis. Studies in humans have focused on applications of amnionic/umbilical cord particles during surgery for OA pain (symptoms) or cartilage defects. However, significant limitations exist regarding comparative controls. It was found, in a well-controlled study, that fillet-derived equine umbilical tissue was safe for use in equine joints and ligaments in the conditions of the study. No adverse events were attributed to the product or the administration of the product. These data support the use of fillet-derived equine umbilical tissue in a clinical safety and effectiveness study in horses with clinical MSK disease of joints, tendons, and ligaments.

The MSK diseased horses in this case study were owned by a research facility which permitted a prospective protocol for quantitative outcomes, baseline values, and a controlled environment. Horses with MSK disease meeting inclusion criteria were brought into box stalls one month before enrollment which minimized the effect of rest on the outcomes. The lameness values, including kinematic data, were similar before and after the month of stall rest. Based on these assessments in a limited number of horses, the administration of microparticulate fillet-derived equine umbilical tissue into OA joints or diseased tendon/ligament was associated with an improvement in pain and lameness scores without evidence of systemic inflammation. Natural case studies, including in humans, often lack matched untreated controls due to the variation in clinical presentation, however, improvement of at least 1 grade in lameness and pain in the target species was observed. Although inconclusive due to the limited numbers of horses, the fillet-derived equine umbilical tissue did not cause them discomfort and may have supported comfort in horses with diseased connective tissues.

The pathology study provided both clinical and histologic evidence of safety for dose escalation and repeat dosing. Since each horse received two fillet-derived equine umbilical tissue injections 5 days before and another allograft injection 2 days before collection of tissues. Health parameters were evaluated for 5 days after injections. Systemically and clinically, the horses were comfortable and with normal twice daily examinations. Synovial fluid protein values were like those reported in the clinically normal horse clinical study. Histologically, synovial membrane and ligament were normal showing lack of response to the allograft. Injection tracts were located and had some minor inflammation. In one injection tract in a ligament, the eosinophilic amorphous tissue filling the tract appeared to be compatible with fillet-derived equine umbilical tissue and showed some minor inflammation, biointegration, and degradative characteristics. This histopathologic finding could not be confirmed with certainty as the fillet-derived equine umbilical tissue. Future studies to identify the material and examine the tissue over a more extended period are warranted.

Following joint administration, a synovitis was anticipated and confirmed to be transient and without clinical signs of pain or outward inflammation and without histologic evidence of synovial membrane reaction. Within the measurements of this study, an immune response to a second administration was not detected in joints or systemically after 3 weeks of exposure from an earlier injection. A transient inflammatory response to pharmacologic or particulate injections into equine joints has been repeatedly reported as a rapid influx of neutrophils that clear unless there is residence and a foreign body reaction. Salt solutions produce an acute neutrophilic synovitis with synovial fluid cell counts to ~8,000 cells/uL and has served as a comparative control including for HA products, dental pulp tissue allografts, and amnion allografts. Substances that contain proteins incite a greater neutrophilic reaction. Importantly, the attraction of neutrophils should be transient and not be associated with infiltration of the synovial membrane by neutrophils or macrophages, indicating a synovial reaction to a substance. In combination, the work with single and multiple joint injections that included synovial fluid analyses and acute histopathology confirm that the neutrophils were passing through from the synovial vasculature to the fluid as a response to the perturbation in the joint fluid and not setting up residency in the synovial interstitium. Synovial lining cells appeared healthy and normal without a histopathologic transition to macrophage phenotype or lymphocyte accumulation. Studies evaluating the joint response to injection with biologics, such as plasma and platelet products and IL-1ra gene therapy have also reported a transient inflammation in the joint fluid. Many studies have not evaluated such an early phase as day 1 after joint injection or compared to saline. In study with synovial fluid analyses, equine isolated mesenchymal stem cells did not increase synovial fluid cell counts over placebo controls or normal ranges; however, baseline values were not reported. The joint fluid cellular inflammation had returned to normal after day 5 and would have been missed in the other reports due to their later days of sampling.

In conclusion, umbilical cord allograft can be characterized as a cryopreserved, off-the-shelf, protein-rich suspension with a well-defined and consistent protein profile both among donors and within samples from a single donor. Many proteins were identified with a structural and protective function in connective tissue supporting use as a connective tissue allograft. Umbilical cord allografts can supplement tissue repair by providing biologic scaffold proteins (fibrillar and gelatinous) to optimize the endogenous tissue environment.

### B. Example 2—Method of Making an Equine Umbilical Tissue Suspension

One example of making a fillet-derived equine umbilical tissue is provided herein. This Example provides a method of making a fillet-derived equine umbilical tissue suspension (referred to herein as equicentCTM). equicentCTM is derived from the equine umbilical cord, prioritizing the intervascular and perivascular connective tissue, which naturally provides structural support and cushioning of the umbilical vessels during fetal development. The primary component of the umbilical tissue is the collagen matrix, which was preserved throughout the processing of equicentC™. This collagen matrix serves as a foundational structure that supports tissue integrity and facilitates interaction with surrounding cells.

The collagen matrix in equicentCTM is a element, providing a scaffold that helps maintain tissue structure and support. This feature is particularly valuable in veterinary applications where the structural integrity of joints and soft tissues is critical, especially in areas that experience high levels of stress or strain (tendons and ligaments). While the naturally occurring growth factors within the connective tissue offers additional benefits, the main function of equicentCTM is to provide a durable structural matrix that supports the body's natural tissue composition and endogenous repair.

A purpose of equicentCTM can be to enhance the performance of the horse by offering structural support to soft tissues and joints under strain, aiding in the maintenance of tissue integrity over time. By providing this foundational matrix, equicentCTM can contribute to the optimization of function and the extension of the horse's ability to perform, ultimately supporting its long-term durability and endurance.

### 1. Methods

The equicenta™ CTM manufacturing process included the collection of equine umbilical cords, dissection, antibiotic treatment, tissue dissociation, straining, and cryopreservation, followed by rigorous quality control (QC) to verify sterility, endotoxin levels, and compliance with predetermined specifications.

i. Tissue Acquisition and Recovery

Healthy looking umbilical cords from healthy foals are collected. The foal is allowed to naturally separate itself from the umbilical cord and the mare naturally passes the placenta before the collection process is started.

a. Initial Collection and Handling

The placenta, umbilical cord, and amniotic membrane are placed into a large bucket filled with water or physiologic salt solution to remove large unwanted debris such as hay and dirt. The umbilical cord is then cut near the base of the placenta.

b. Rinsing and Initial Disinfection

Once separated from the placenta, the umbilical cord is placed in a bucket of sterile 0.9% NaCl (sterile water would work too) to remove visible debris (Wash #1). After rinsing, it is submerged in half-strength Dakin's solution for 30 seconds but no more than 60 seconds to ensure initial disinfection (Wash #2). The use of sterile saline and Dakin's solution (a Sodium Hypochlorite (bleach) solution) allows for removal of both physical contaminants and bacteria from the tissue. The amnion outer layer acts as a sleeve and protects the umbilical tissue from external solutions.

c. Antibiotic Solution

Following the initial disinfection, the tissue is placed in an antibiotic transport solution to further disinfect the tissue during storage and shipping (Wash #3). A sterile container is filled with 1L 0.9% NaCL for irrigation (0.9% NaCl for Injection would work too) and 20 mL of Anti-Anti (100×) (active ingredients of Anti-Anti are Penicillin G, Streptomycin, and amphotericin B) to create the antibiotic solution. This solution is used to provide protection against bacterial growth.

A formulation of the antibiotic solution provides a 2% Anti Anti in Saline solution is used whereas the industry standard for a working concentration of anti anti is 1%. The 2% solution serves as a stronger barrier to minimize bacterial contamination during tissue recovery.

d. Storage and Shipping —

Once the tissue is properly recovered, it is stored in a refrigerator until ready for transport. The tissue is shipped in an insulated container using a tracked expedited process with ice packs to maintain a consistent temperature and prevent any degradation.

ii. Donor Selection Criteria, Veterinarian Physical Exam and Sample Collection

To qualify as an acceptable donor, mares met the following health and age requirements:

a. Health Status:

Donors must be free from clinically observable diseases, particularly those that could affect tissue quality or pose a risk to the safety of the final product. Mares should have a history of normal foaling and be in good health at the time of donation.

b. Health and Behavioral Assessments:

A physical examination by a licensed veterinarian ensures that the mare and foal is free from any visible signs of illness, including screening for both infectious and non-infectious conditions that could affect tissue suitability. Assessments listed below are examples but may include other pathogens or parameters.

c. Age: Mares selected for tissue donation are typically of an age range of 4-25 years old.

Diagnostic Testing Laboratories can be used to test for the following diseases:

### (A) Mare—Equine Arteritis Virus (EAV)—

EAV is a highly contagious virus that can cause reproductive issues in mares, such as abortion or infertility. It is also capable of causing systemic illness and respiratory signs in horses. Testing for EAV can help prevent the transmission of this virus through tissue collection, as it could compromise the quality of the tissue and its suitability for processing. The virus can also spread easily between horses in close proximity, making it necessary to screen for EAV to ensure the donor tissue is free from this risk.

### (B) Mare—Equine Herpesvirus-1 (EHV-1)—

EHV-1 is a viral pathogen that can cause a wide range of symptoms, from respiratory infections to neurological disease and abortion Since EHV-1 can affect the nervous system and reproductive organs, testing can be used to prevent any risks related to infected tissue. Ensuring that mares are free of this virus helps maintain tissue integrity and safety during processing.

### (C) Mare—Equine Herpesvirus-4 (EHV-4)—

EHV-4 primarily causes respiratory issues in horses but can also lead to secondary complications such as pneumonia.

### (D) Mare—West Nile Virus (WNV)—

WNV is transmitted by mosquitoes and can affect the central nervous system, causing symptoms such as fever, ataxia (lack of coordination), and even death. In horses, WNV can cause neurological disease, leading to permanent disability or death in severe cases. Screening for WNV can help ensure that tissue from affected mares is not collected, as this virus can significantly impact the health of horses and their ability to survive long-term.

### (E) Mare—Equine Infectious Anemia (EIA) (Coggins Test)—

EIA is a viral disease transmitted by biting insects or contaminated equipment. It causes anemia, fever, and swelling, and infected horses can develop a chronic or acute form of the disease. EIA is highly contagious, and infected horses are subject to quarantine or euthanasia to prevent further spread. The Coggins test is the industry-standard method for detecting EIA and can be used to prevent any potential transmission through tissue collection.

### (F) Mare—Eastern Equine Encephalitis (EEE)—

EEE is a mosquito-borne virus that affects the central nervous system in horses. It can cause symptoms ranging from fever to neurological deficits and, in many cases, can be fatal. While it is a rare disease, the consequences of an EEE infection can be devastating. Testing for EEE helps prevent the collection of tissue from mares potentially carrying this virus, ensuring that the tissue used in equicenta™ CTM is free from serious health risks.

### (G) Foal—Equine Parvovirus—

Equine Parvovirus is a common virus found in horses, often in a latent or subclinical form. It has been detected in the majority of horses at some point in their life, and, while it may not present symptoms in many horses, it is still helpful to test for it as part of the donor screening process. Testing for parvovirus can help in ensuring that the tissue is free from any detectable viral load that could compromise tissue safety.

iii. Receipt of UC

All umbilical cords processed into equicenta™ CTM can be received at the processing facility at a temperature of 0-10 °C.

If the tissue passes visual inspection and all documentation is found to be acceptable, it can either be stored in the refrigerator for short-term storage or immediately transferred to the cleanroom for processing, where it can be used in the subsequent processing steps.

Processing (dissection) can be conducted within 72 hrs from when the foal is born, or preservation procedures may be followed for later dissection.

iv. Dissect Umbilical Cord
a. Tissue Handling—

Once preparation is complete, the umbilical cord was carefully removed from its transport container.

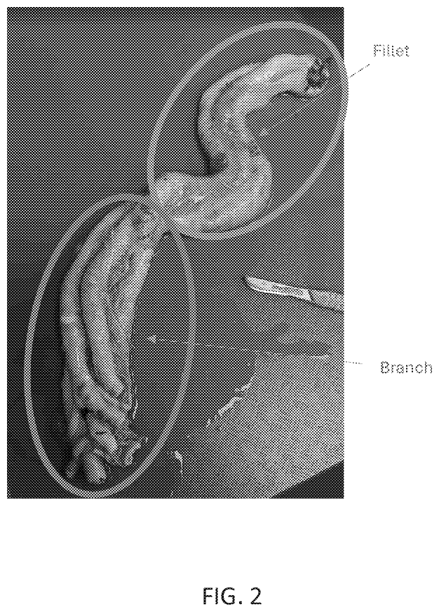

During dissection the “fillet” is separated from the “branch” tissue (See FIG. 2).

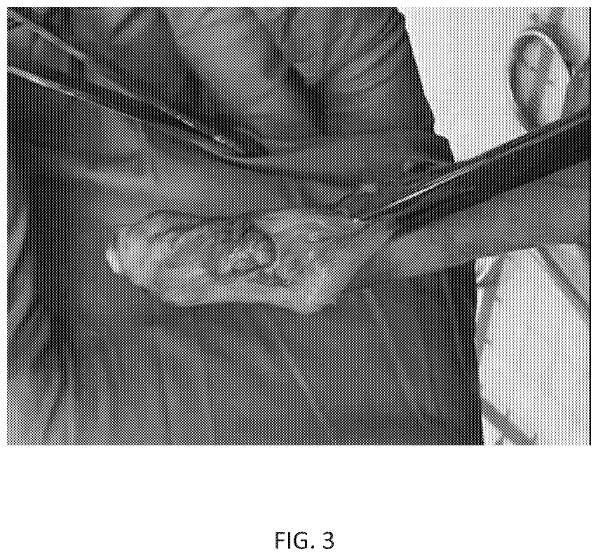

Then the surrounding amnion membrane layer is removed which oftentimes has some hay, hair, dirt and hippomanes (hippomane in FIG. 3).

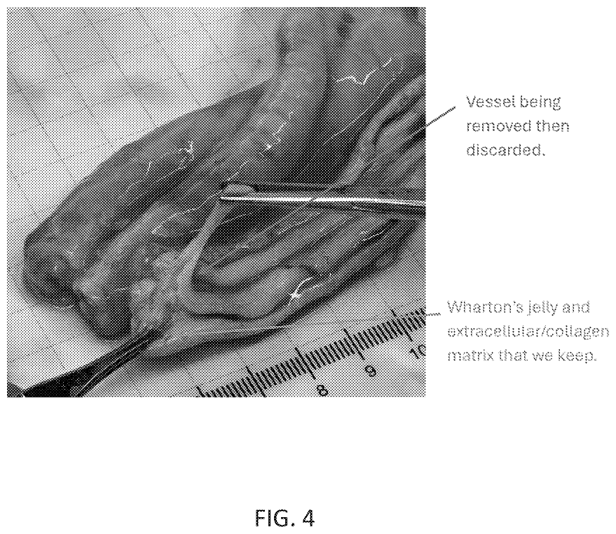

Following the removal and cleanup of the amnion, scissors are used to cut open the vessels and then the vessels are stripped away and discarded as shown in FIG. 4.

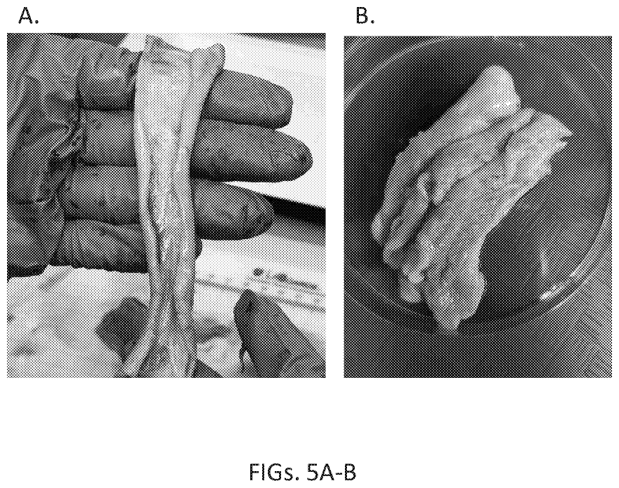

After the removal of the vessels the tissue is carefully inspected to ensure that only the intervascular and perivascular connective tissue remain, while any residual blood vessels or unwanted tissue is discarded. Refer to the image on the left of FIG. 5, showing isolated perivascular layer and on the right of FIG. 5 the isolated intervascular and perivascular layers as a whole.

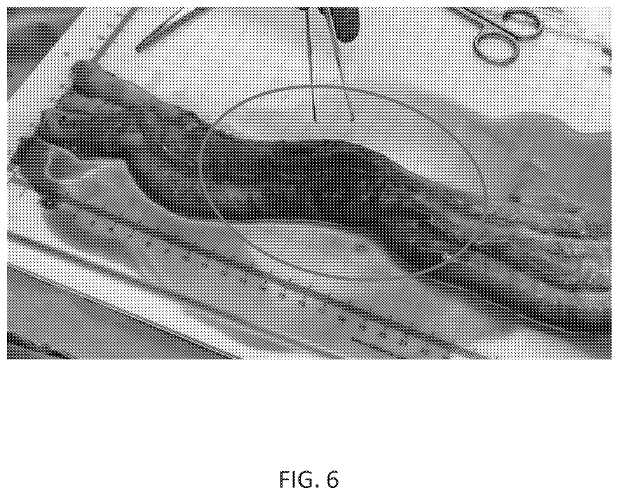

Sometimes cords have blood clots and red stretch marks, the red, bloody looking tissue is cut/removed. FIG. 6 shows an example of red stretch marks that are cut away. Oftentimes this is a superficial stretch mark but sometimes it is deep so the whole section is removed. As little blood cells in the final product as possible is desired.

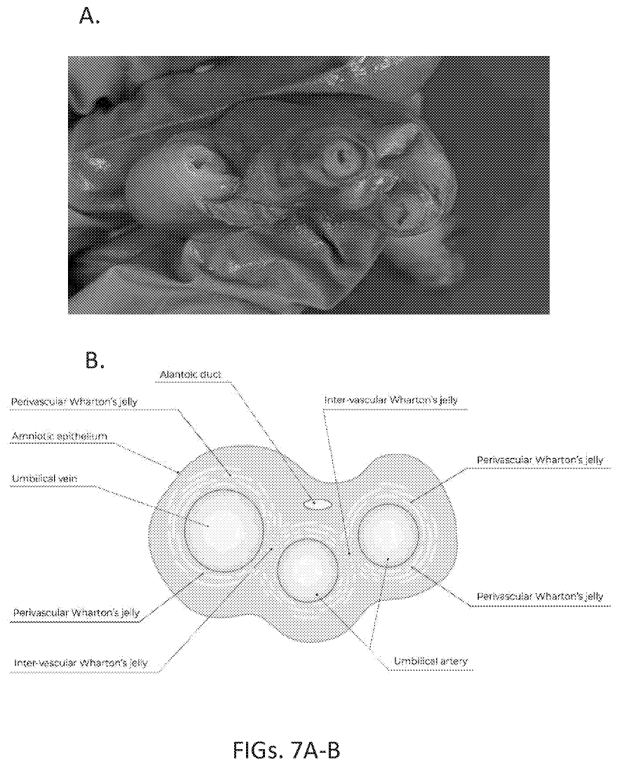

FIG. 7 shows a cross section of the “fillet” part of the cord and also a cartoon illustration to explain the anatomy of the equine umbilical cord.

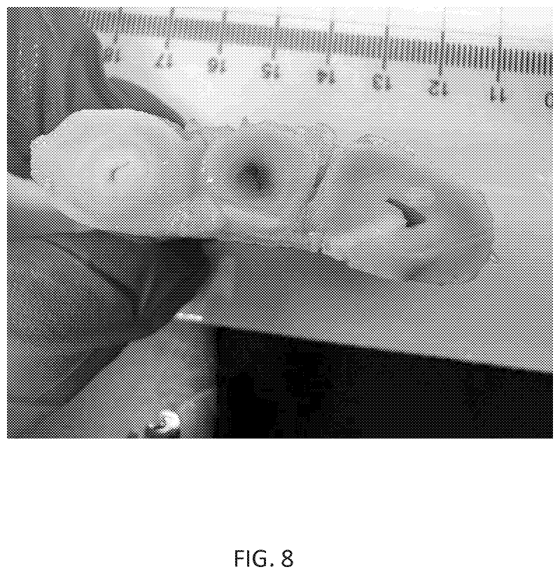

FIG. 8 illustrates a cross-section of the fillet used in the histology study, showing what can be described as three tubes connected by connective tissue. The tubes consist of the umbilical arteries on the left and the umbilical vein on the right. Each tube is characterized by an inner ring and an outer ring. The innermost ring is composed of endothelial and mesothelial tissue, which forms the actual vessel, while the outer portion of the ring consists of perivascular connective tissue, which appears more solid in texture. The connective tissue holding these structures together is the intervascular portion, which has a more gelatinous consistency

v. Antibiotic Soak

After dissection and isolation of the FDUT, the next step is antibiotic treatment. The purpose of this step is to ensure that the tissue remains sterile and free from bacterial contamination before it undergoes dissociation.

a. Preparation of Antibiotic Solution—

In a 500 ml container, the antibiotic solution is prepared by combining sterile saline with Anti-Anti 100× (antibiotic concentrate). 5 mL of antibiotic is added to 250 ml of sterile saline for irrigation creating a 2% solution.

b. Soaking the Tissue—

The tissue is then soaked in this 2% antibiotic solution for a specified time (30 minutes up to 24 hours). This treatment reduces the microbial load on the tissue, ensuring that it remains sterile during subsequent processing steps.

c. Rinsing the Tissue—

After the soaking period, the tissue is removed from the antibiotic solution and transferred to a new 500 ml container filled with sterile saline to rinse away any remaining antibiotic. This step serves two purposes: 1) Avoiding antibiotic presence in the final product, as antibiotics can cause cytotoxicity. 2) Preventing a false positive in the bioburden sample.

d. Bioburden Testing—

Bioburden sample is also collected at this stage to test for any residual microorganisms, which further ensures that the product meets sterility standards. The saline rinse (approx. 60 mL) from the second container is then collected in a smaller 100 ml container and shipped off to a third party for analysis. Results also provide data on the efficacy of the antibiotic treatment.

The tissue can then be broken up further for dissociation.

vi. Tissue Dissociation

Following the antibiotic soak, the tissue is rinsed in clean 0.9% NaCl, weighed and then placed in the Auxocell system to grind the tissue into small pieces (approx. 1 mm pieces). A scalpel or scissors can be also used to cut the tissue into the small pieces needed.

Mechanical dissociation allows the tissue to retain its original structural properties while breaking it down into smaller, finer pieces, in compliance with Animal Cell Tissue Product (ACTP) guidelines. This helps minimize the alteration of the tissue's natural structure and maintains its suitability.

### Mechanical Dissociation of Tissue

The process involves mechanical dissociation to reduce a tissue sample into a flowable, injectable suspension of fine particulates while minimizing alteration of the tissue's natural structural properties, in compliance with Animal Cell Tissue Product (ACTP) guidelines. The dissociation is generally performed in two stages: an initial coarse dissociation followed by a fine dissociation or homogenization.

ii. Coarse Tissue Dissociation

Following an optional antibiotic soak and rinse in a suitable buffer such as 0.9% NaCl, the tissue undergoes a coarse mechanical dissociation to reduce it into smaller pieces. A target size for these pieces may be approximately 1 mm.

In a particular embodiment, this initial dissociation is performed using an Auxocell system. The tissue is weighed and then processed by the system to achieve the desired particle size. Alternatively, this step can be accomplished through other aseptic methods, including, but not limited to, manual mincing with a scalpel or scissors, or processing with a tissue mill. The primary objective of this stage is to prepare the tissue for the subsequent fine dissociation step.

### Fine Tissue Dissociation and Homogenization

Following coarse dissociation, the smaller tissue pieces are further processed into a fine, flowable suspension suitable for straining and injection. This is achieved by subjecting the tissue pieces to further mechanical forces in a suitable liquid medium.

In a preferred embodiment, this fine dissociation is performed using a gentleMACS™ Octo Dissociator with corresponding gentleMACS™ C-Tubes. The coarse tissue pieces are placed into a C-Tube with a sterile saline solution. A ratio of approximately 3 mL of tissue to 7 mL of saline has been determined to be effective, as it provides an easily strainable material with minimal foaming, thereby optimizing material quality and process costs.

In this exemplary embodiment, the C-Tubes are subjected to a dissociation cycle on the gentleMACS™ device. The device may be configured with a custom program that controls parameters such as rotational speed (RPM), direction, pulse, and acceleration to optimize the process. An optimized cycle time of approximately 2 hours has been identified to provide an ideal balance between efficient dissociation and the preservation of tissue integrity, including cell viability and protein structure.

The selection of a 2-hour cycle in this embodiment was determined through experimentation. It was observed that a shorter 1-hour cycle resulted in a suspension that was too thick for efficient straining, while a longer 3-hour cycle led to excessive mechanical forces, causing a decline in cell viability and an increase in protein degradation. The 2-hour cycle mitigates these issues.

It is to be understood that the gentleMACS™ system is one example of a suitable technology for this step. Other methods for achieving fine dissociation and homogenization can be used. These alternative methods include, but are not limited to, the use of a tissue micronizer, tissue homogenizer, or other suitable mechanical blending or milling devices that can render the tissue into a minimally manipulated, flowable form for its intended use.

vii. GentleMACS Grind

Once the tissue is resized into the small pieces, then the tissue is placed into a gentleMACS C-tube at approximately a 3 mL tissue to 7 mL saline for injection ratio. This ratio has been found to be successful in providing easily strainable material while minimizing the number of C-tubes needed, thus saving on process costs. Although other ratios have been experimented with in the past, the 3:7 ratio offers the best balance between flowability (less foam produced) and material quality for the final product. Then the c-tubes are subject to a minimum 2 hr grind on the gentleMACS Octo dissociator from Miltenyi Biotec. There is a custom program built into this machine which adjusts the RPM, clockwise, counterclockwise, pulse, ramp up rotation, etc.

Cycle Time Optimization—A 1-hour cycle per C-tube on the GentleMACS can be used. This cycle time resulted in a thicker suspension, making straining difficult and time-consuming.

Alternatively, applying a 3-hour cycle per C-tube introduced excessive mechanical forces, leading to a decline in product cell viability and an increase in protein degradation.

Based on these observations, a 2-hour cycle was determined to provide the ideal balance between efficient dissociation and maintaining tissue integrity. This cycle duration ensures that the tissue is broken down into fine particulates while minimizing cell damage and protein degradation.

In addition to the Auxocell and GentleMACS dissociator, other known aseptic cutting or chopping methods can be used to break down the tissue. These include the use of scalpels, scissors, Micronizer, Tissue mills or any other suitable mechanical (i.e., blender) method that ensures the tissue is minimally manipulated and remains in a flowable form suitable for injection.

viii. Straining and Filtration

Purpose of Straining—The goal of straining is to remove any larger, undesirable particulates or residual tissue that were not properly broken down during the dissociation process. By using sterile mesh, screen, or sieve filters, the straining process helps to ensure that the final product has uniform particle size that allows for easy injection via syringe, meets quality standards for consistency, flowability in clinical applications, and is free from any undissociated clumps or large debris that could hinder the product's functionality.

Straining Procedure—The tissue suspension, which consists of fine particulates of FDUT mixed with saline, is poured through a series of sterile mesh filter screens or sieves. The liquid and appropriate size particles are allowed to pass through. Any larger particles that do not pass through the mesh are collected in the filter and discarded. The mesh size is selected based on the desired particulate size and the specifications for the final product.

A series of screens are used to progressively filter the tissue, starting with a 1000 μm mesh to remove the largest particles. The suspension is then passed through progressively smaller screens: 500 μm, 300 μm, 200 μm, and finally 100 μm. This step-by-step process ensures that clumping is minimized and that as much usable tissue as possible is retained in the suspension.

Previously, a 300 μm particle size with a 75 mg/ml concentration was developed. Safety studies indicated that the particulate suspension was received in unfavorably in some joints of a small number of horses, raising safety concerns of that formulation in joints in horses. Reducing the particle size to 100 μm, chilling during processing to reduce foaming, and a final concentration of 50 mg of tissue/ml improved the biocompatibility and tolerability of the product in horses' joints. In ligaments, however, the 300 μm particle size with a 75 mg/ml concentration as the final product was well tolerated. Further studies have confirmed that the 100 μm particle size was safe, and 50 mg of tissue/ml concentration was biocompatible including in diseased joints and ligament, with no adverse reactions observed.

Collection of Strained Material—The filtered suspension, now containing only the finely dissociated tissue, is collected in a clean, sterile container.

Calculation of required volume—Target concentration for the final product is approximately 50 mg/ml, that is 50 mg of tissue process into 1 ml total volume. To make sure the final concentration from 50 mg of tissue/ml is obtained, the final total volume will be based on the starting weight of the tissue collected after dissection is calculated.

$$Total Final Volume ( mL ) = \frac{( UC Weight ( mg ) ) * 1 mL}{Desired mg / mL} \qquad Formula$$

Using the beginning tissue “fillet” weight, calculate the Total Final Volume allowed to achieve the 50 mg/mL and 5% DMSO concentration using the equation below.

ASSUMPTION: 1 g Tissue=1 mL Tissue

Next, the total final volume is divided by two to obtain the half volume. This half volume represents the expected volume of the tissue suspension after the straining process (before cryoprotection).

If the actual volume is found to be below the calculated half volume, processing personnel must QS (Quantity Sufficient), meaning they should add enough 0.9% NaCl for injection to reach the desired calculated (half) volume.

When the actual volume exceeds the half volume adjustments need to be made to ensure that the final total volume is reached while maintaining the 50 mg/mL concentration of tissue and the 5% DMSO (there are other ways to cryoprotect, but DMSO was better) concentration in the final product.

ix. Cryoprotect and Vial

Prior to vialing, 10% DMSO solution is prepared. The tissue suspension is then added in a 1:1 ratio to the 10% DMSO solution; however best practice is to not combine more than 50 mL of 10% DMSO and 50 mL of tissue suspension at one time in order to prevent the DMSO from sitting with the tissue for too long and warming up.

The 1:1 ratio of 10% DMSO and tissue suspension (approx. 100 mg tissue per mL volume) achieves the final target values for equicenta™ CTM: Tissue Concentration: Approx 50 mg/mL; DMSO Concentration: 5% final volume of DMSO; Total Final Volume: as calculated from the total tissue mass after accounting for tissue losses.

Vialing—Dispense equicenta™ CTM into the sterile cryovials and pass the vials out of the BSC where non-sterile technician(s) will put the vials into a cryobox and place in the −20 °C Freezer in order to keep the vials cold during the remainder of the vialing process.

x. Sterility and Endotoxin Testing
a. Sterility Testing

Sterility testing confirms that the final product is free of microbial contamination, including bacteria, fungi, and yeast, ensuring it is safe for clinical use.

Methodology: Samples of the final vialed product are tested using standardized microbiological techniques, typically involving incubation in appropriate growth media under controlled conditions to detect microbial growth.

b. Endotoxin Testing

Endotoxin testing assesses the level of bacterial endotoxins (lipopolysaccharides), which can cause severe inflammatory reactions. Ensuring low endotoxin levels is critical for patient safety.

Methodology: Samples are tested, and results are quantified against known standards to determine the level of endotoxins present.

### C. Example 3—Exosomes

Table 10 shows an exosome data snapshot: data was generated on CD9 capture chips using NanoView SP-IRIS. Particles (50-200 nm) were counted and then fluorescence was used to confirm phenotypes. For each sample, total count is every particle detected on the CD9 spot; the CD9-only, CD81+CD9, CD90+CD9, and CD90+CD81+CD9 columns partition those fluorescently confirmed events. |Sum CD9+| adds those bins, and % CD9+=(|Sum CD9+|/Total count)×100 reflects capture specificity. Because counts are reported in ×10⁸ particles, the |Sum CD9+| values correspond to hundreds of millions to multi-billion CD9⁺ exosomes per sample (values≥10 in this unit=billions), demonstrating that the preparations contain billions of exosomes while maintaining high CD9 specificity across the batch (~1 on average).

TABLE 10
Exosome Counts (×10⁸ particles) from FDUT Supernatant
on CD9 Capture (SP-IRIS), with CD81/CD90 Co-Localization

| | Total | | | | CD90 + | | |
|---|---|---|---|---|---|---|---|
| Sample | Count | CD9- | CD90 + | CD81 + | CD81 + | Sum | |
| ID | (50-200 nm) | only | CD9 | CD9 | CD9 | CD9⁺ | % CD9⁺ |
| EPL24- | 8,900 | 4,475 | 39 | 3,159 | 30 | 7,703 | 86.6% |
| 002-084 | | | | | | | |
| EPL24- | 3,292 | 2,497 | 2 | 651 | 1 | 3,151 | 95.7% |
| 002-085 | | | | | | | |
| EPL24- | 5,716 | 2,965 | 13 | 2,055 | 14 | 5,047 | 88.4% |
| 002-086 | | | | | | | |
| EPL24- | 7,605 | 5,607 | 2 | 1,141 | 2 | 6,752 | 88.8% |
| 003-031 | | | | | | | |
| EPL24- | 2,842 | 2,068 | 4 | 634 | 3 | 2,709 | 95.3% |
| 004-031 | | | | | | | |
| EPL24- | 1,414 | 1,064 | 20 | 265 | 10 | 1,359 | 96.1% |
| 005-031 | | | | | | | |
| EPL24- | 7,437 | 3,916 | 32 | 2,553 | 39 | 6,540 | 87.9% |
| 007-031 | | | | | | | |
| Batch 1 | — | — | — | — | — | — | 91.2% |
| average | | | | | | | |

Table 10 Counts are reported as N × 10⁸ particles per mL after correcting for the 1:_ dilution, e.g., a value of 7.7 corresponds to ~7.7 × 10⁸ (~770 million) CD9⁺ particles per mL

Exosome content is known to contain cell cytosol (cytoplasm). Known contents of pro-regenerative value include some mRNA, proteins, and growth factors. Table 1 demonstrates that FDUT suspension contains billions of exosomes from umbilical tissue. FDUT cells are from foals, so the cells are mesenchymal and have stem cell characteristics as shown in our exosome data. The CD 90 marker is an established marker of stem cells and a portion are co-localized on the CD9 marker that denotes an exosome. As an example, the first EPL-002-084 has 39×10⁸ (millions) of exosomes from stem cells. For a given cell the CD markers reflect a small portion of the plasma membrane, so this reflects a minimum number; the number that happened to enclose the CD90 stem cell marker onto the plasma membrane of the exosome.

### D. Example 4—Microscopy of Fresh Thawed and Biodegraded Cryopreserved Equine Umbilical-Derived Tissue Allograft (equicenta™CTM): A Descriptive Analysis

### 1. Introduction:

Microparticulate bio-scaffolds have the potential to serve as structural support during the repair of injury. The use of fetal and placental tissue as a source of tissue allograft has been explored and offers a potential framework for structural support and foundational proteins for healing, including growth factors, glycosaminoglycans (GAGs), glycoproteins, and exosomes. Fetal origin confers immune tolerance, and, through proprietary processing, these primordial tissues can be prepared with minimal manipulation for homologous use.

Numerous studies have evaluated biologic scaffolds as grafts for injuries to joints, tendons, and ligaments. These biological grafts offer the benefits of biocompatibility and biodegradability. Equicenta™CTM is an equine umbilical cord-derived tissue suspension that has recently been evaluated in vivo and shown to be safe and biocompatible for intra-articular use in horses. This product contains thousands of fetal proteins with key properties to support tissue healing.

These studies can microscopically assess 1) fresh-thawed cytospin smears of microparticles for structure and contents and 2) evaluate two enzymatic digestion techniques to confirm that equicenta™CTM is biodegradable with tissue enzymes, demonstrating the potential for residual release of matrix proteins and natural turnover in vivo.

### 2. Methods:

Cryopreserved umbilical-derived product, equicenta™CTM, was thawed at room temperature and pelleted by microcentrifugation at 10,000 rpm. The supernatant was discarded and the pelleted product was applied to charged glass slides. Slides were fixed with a 1:1 dilution of cold acetone and methanol for 10 minutes. Slides were stained with either Diff Quik or hematoxylin for microscopic assessment at 10× and 20× power.

For the second part of the study, vials of cryopreserved equicenta™CTM were thawed as described for clinical use and exposed to one of the three treatment groups: 1) Saline, 2) 50 U/ml of collagenase I; 3) 0.25% trypsin.

The media or digestive enzyme was incubated with the product at 37 °C for 30 minutes (trypsin) to 1 hour (media only and collagenase group) in a 15 ml conical tube. After incubation, the tubes were centrifuged at 300×g for 10 minutes. The supernatant was discarded and the pelleted product was resuspended in a T-25 flask with 5 mls of culture medium. Phase contrast images were taken using the EVOS® XL (Life Technologies; Carlsbad, CA) at 10×, 20×, and 40×, at various fields of view to document the product in media.

### 3. Results

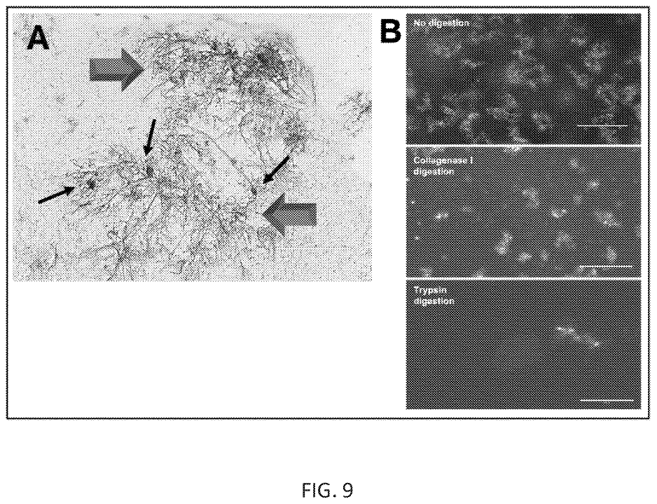

Filamentous matrix particles were observed in the cytological evaluation of a pelleted umbilical-derived tissue allograft, with background pink staining indicating a proteinaceous extracellular matrix surrounding the net-like scaffolding structure (FIG. 9A). Equicenta™CTM in culture media without digestion displayed aggregates of extracellular matrix that did not attach to the plastic flask and floated within the fluid, as would be anticipated for matrix particles. After enzymatic digestion with collagenase, the aggregates were smaller and fewer, indicating digestion of collagens and a reduction in particulate formation. Trypsin digestion of equicenta™CTM was almost complete, where only a few particle aggregates remained (FIG. 9B).

### 4. Discussion:

Equicenta™CTM is a biological, filamentous-like scaffold with an associated extracellular matrix can provide both a physical network for endogenous cell migration and, with the known growth and development proteins, also to recruit and support cell residency during healing. Microscopic visualization of equicenta™CTM in culture confirmed the presence of micron particles that can coalesce when in proximity. Collagenase partially digested the bioscaffold matrix of equicenta™CTM in culture, as evidenced by less product clumping and smaller particulate aggregates compared to the undigested samples. Collagen is an abundant constituent of equicenta™CTM. As this collagenase is naturally present in tissue, these findings indicate that equicenta™CTM would undergo natural biodegradation by enzymes in vivo, particularly in injured tissue. A lower number of particulate aggregates was observed after digestion with 0.25% trypsin, indicating a more extensive breakdown of the product scaffold matrix than with collagenase. Trypsin is commonly used to dissociate cells from each other and from the extracellular matrix, and for equicenta™CTM represented a complete and robust digestive agent for this bioscaffold product.

Further analysis of equicenta™CTM protein properties during digestion and more in-depth microscopic evaluation can characterize the product's capacity to serve as a reservoir of anabolic, anti-catabolic and anti-inflammatory proteins.

In conclusion, equicenta™CTM provides a biological extracellular matrix with a scaffold structure that can be used for endogenous tissue healing in orthopedic injuries. Additionally, this extracellular matrix was processed by naturally occurring proteolytic enzymes found in joints, tendons, and ligaments, which, when activated, can release additional proteins used for repair.

### E. Example 5—Nanoparticle Tracking Analysis of Equine Cryopreserved Umbilical-derived Connective Tissue Allograft (equicentC™) Using Exoview to Determine Presence and Quantity of Exosomes

### 1. Introduction:

To isolate and characterize extracellular vesicles (EVs) from equine umbilical cord tissue suspensions, confirming particle concentration, size distribution, and suitability for downstream exosome validation workflows.

The objectives of this study were to 1) isolate extracellular vesicles (EVs), including exosomes, from equine umbilical cord tissue suspensions using differential ultracentrifugation; 2) characterize the isolated EVs for particle concentration and size distribution using Nanoparticle Tracking Analysis (NTA), verifying alignment with the exosome-enriched size range (50-200 nm) to confirm suitability for downstream analysis; 3) determine the presence of tetraspanin proteins (e.g., CD9, CD63, CD81) typically associated with exosomes, confirming exosome identity in alignment with ISEV minimal characterization guidelines; 4) determine the relative amounts and distribution of key exosomal surface proteins and other relevant markers among different equine donors within the Equicenta™ CTM pipeline; and 5) assess the variability of exosome-associated proteins among samples obtained from a single donor, supporting the evaluation of intra-donor consistency for quality control and product development.

### 2. Materials And Methods:

a. Exosome Isolation

One and a half milliliters of equine umbilical cord suspension were transferred to a 1.5 mL microcentrifuge tube and centrifuged at 2,000×g for 10 minutes at 4 °C using a Beckman Microfuge® 22 benchtop microcentrifuge (Beckman Coulter). The resulting supernatant was transferred to a 33.5 mL thick-wall polycarbonate tube (13×51 mm, Beckman Coulter, catalog number 349622) and subjected to ultracentrifugation at 35,600 rpm for 90 minutes at 4 °C using a SW55 Ti rotor (k-factor 48 at maximum speed) in an Optima L-90K ultracentrifuge (Beckman Coulter).

The pellet was resuspended in 800 μL of Dulbecco's Phosphate-Buffered Saline (DPBS) filtered through a 0.1 μm Minisart® PES syringe filter (Sartorius, code 16553------K). The resuspended sample was then transferred to a suitable ultracentrifuge tube and centrifuged at 65,000 rpm for 90 minutes at 4 °C using a TLA-100.2 fixed-angle rotor (k-factor 13 at maximum speed) in an Optima TLX-120 benchtop ultracentrifuge (Beckman Coulter).

Following this second ultracentrifugation step, the final pellet (P100) was resuspended in 100 μL of 0.1 μm-filtered DPBS for downstream characterization and analysis.

b. Nanoparticle Tracking Analysis

To confirm exosome identity and evaluate surface marker profiles, isolated vesicles were analyzed using the NanoView ExoView® platform (NanoView Biosciences). Samples were diluted according to manufacturer guidelines to achieve optimal particle loading on ExoView chips.

ExoView Tetraspanin Chips pre-coated with antibodies against CD9, CD63, and CD81 were used for particle capture. Diluted samples were incubated on the chips overnight at room temperature to allow for exosome binding.

Following incubation, chips were washed with the provided buffer to remove unbound material. Fluorescently labeled detection antibodies (typically against CD9, CD63, CD81, and additional markers of interest such as CD105 and CD90) were applied to enable multiplexed marker detection on captured particles.

After washing to remove excess detection antibodies, chips were imaged using the ExoView R100 reader. Interferometric imaging provided single-particle sizing and counting, while fluorescent imaging enabled marker-specific profiling and co-localization analysis on individual particles.

Data were analyzed using ExoView Analyzer software, providing: total captured particle counts; size distribution of captured particles; and presence and relative abundance of tetraspanin and additional markers on single particles, confirming the identity of the isolated extracellular vesicles as exosomes in alignment with ISEV guidelines.

### 3. Results

13 equine umbilical cord tissue exosome samples were analyzed using the NanoView (ExoView) platform. This system uses microarrays of antibodies to capture exosomes bearing specific surface markers, then fluorescent detection antibodies to identify which markers each exosome carries. Five key exosomal surface markers—CD9, CD81, CD63 (classic exosome tetraspanins), and CD90, CD105 (MSC markers)—were focused on for measuring the particle counts and co-expression of these markers across all samples. The findings for each marker, including the fraction of exosomes positive for that marker and any co-expression with other markers is described herein.

### 1. CD9 (Tetraspanin)

### 1. Particle Counts: CD9 (Tetraspanin)

CD9 is a canonical exosome membrane protein and was strongly detected in every sample. The NanoView bar graphs (“Particle Counts—Capture Probe: CD9”) show CD9 captures at ~10⁸-10⁹ particles/mL, far exceeding the isotype (MIgG1) control. From the colocalization tables, CD9-only exosomes account for approximately 50-70% of the total particles in batch 1 (e.g., 4,450 out of 8,900 for EPL24-002-084), and when combined with co-expressed markers, CD9-positive vesicles represent roughly 90% or higher of the exosome population. This confirms that the majority of our EVs display CD9 on their surface.

a. Co-Expression:

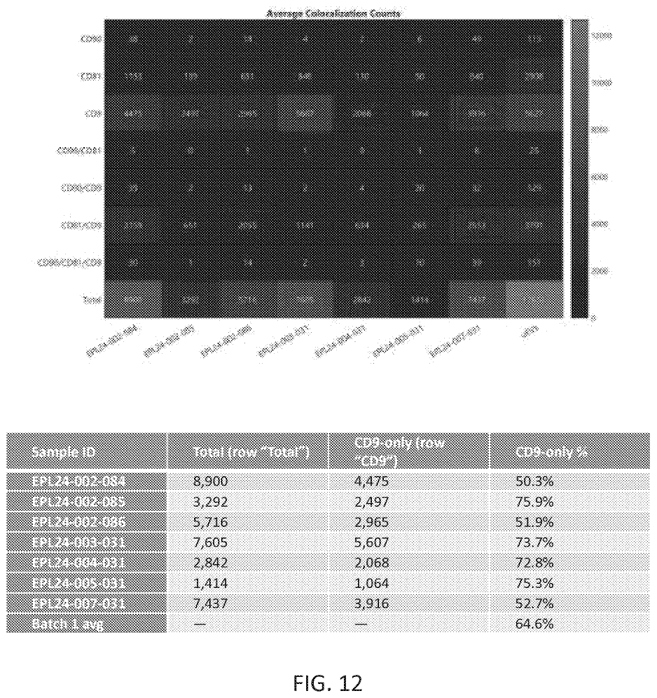

Many of the CD9-bearing exosomes also carried other tetraspanins. According to the NanoView co-localization heatmaps (FIG. 12), CD9+CD81 double-positive vesicles account for 40-60% of the total exosome population in each sample (e.g., 3,159 of 8,900 or ~36% in EPL24-002-084 up to 2,553 of 7,437 or ~34% in EPL24-007-031). In other words, a single exosome often had both CD9 and CD81 on its surface. A majority of CD9-positive vesicles (on average ~90%) carried at least one other tetraspanin (CD63 or CD81), indicating these markers tend to appear together on the same exosomes. Visually, the combination heatmap shows large intersections in the CD9-CD81 bins, confirming that CD9 and CD81 are frequently co-expressed.”

### 2. CD81 (Tetraspanin)

a. Particle Counts:

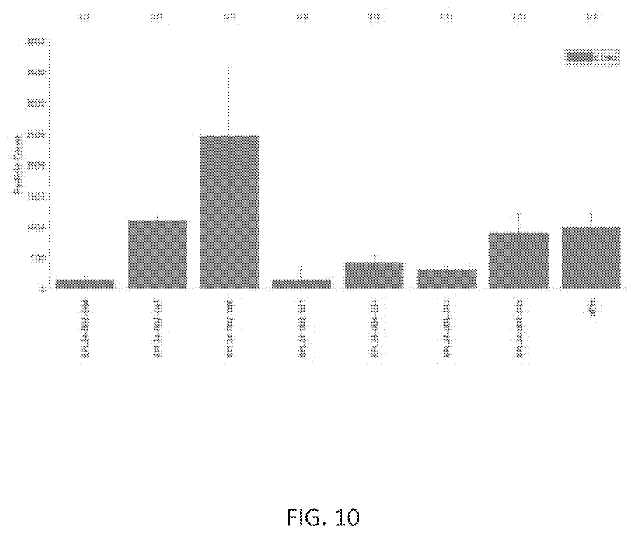

CD81 is another classical exosomal tetraspanin, and it was also abundantly detected. In the NanoView assays, a high number of exosomes bound to anti-CD81 capture spots, indicating widespread expression of CD81. Bar graph results (FIG. 10) for CD81 capture were comparable to CD9 in magnitude, and well above the background control, confirming that many of the vesicles carry CD81. Across all samples, around 10-15% of exosomes were CD81-positive. CD81-positive particles thus represent a significant subset of the population, although slightly fewer in number than CD9-positive ones (CD9 tended to yield higher counts than CD81 in some samples). Still, roughly one in ten exosomes displayed CD81 on its surface in these equine samples.

b. Co-Expression:

CD81 was frequently co-expressed with CD9 and/or CD63 on the same exosomes. The NanoView co-localization analysis confirms an overlapping sub-population of **CD9+/**CD81+ exosomes. Many exosomes captured via CD81 also lit up for CD9 detection, showing they had both markers. Likewise, exosomes captured on CD9 spots were often positive for CD81 upon fluorescent staining. The reports included a co-expression chart (effectively a heatmap of marker overlap) that illustrates combinations like “CD9-CD81” and “CD81-CD90,” demonstrating these overlaps. There was also some **CD81+/**CD63+ co-expression (since CD63 is another tetraspanin), though CD63 was not assessed by fluorescence in the main run (see CD63 section). In summary, CD81 and CD9 commonly appeared together on exosomes, indicating many vesicles carried multiple tetraspanin markers.

Visually, one can see confirmation of CD81 positivity in the report's summary graphics—for example, a heatmap (FIG. 12) of marker co-localization shows a prominent intersection between CD9 and CD81 signals, underlining that a large fraction of CD81-bearing exosomes also have CD9. This co-expression of tetraspanins is typical for exosomes and supports the validity of the assay.

### 3. CD63 (Tetraspanin)

a. Particle Counts:

CD63 is the third canonical exosomal tetraspanin examined. According to the NanoView particle counts, CD63 had the highest capture of particles among the tetraspanins, indicating that a majority of exosomes were CD63-positive. In fact, many exosomes that did not have CD9 or CD81 still bound to the CD63 capture spots. The bar charts (FIG. 10) show very strong exosome binding on anti-CD63 spots (significantly above the isotype control), implying that CD63 is widely present. By inference, well over half of all exosomes carried CD63. (This aligns with the observation that only ~24% of exosomes showed CD9 or CD81 in detection—the remainder were predominantly CD63-positive exosomes lacking the other two markers.)

b. Detection Challenges:

Notably, direct fluorescent detection of CD63 was attempted in a study but was unsuccessful due to an ineffective antibody clone. Thus, in the main NanoView runs a CD63 detection signal was not present. Instead, capture data can be relied on to assess CD63. The inability to stain CD63 means that exosomes captured via CD63 appeared “negative” for the other markers if they carried only CD63. This explains why a large fraction of exosomes (~76%) were recorded as negative for CD9/CD81/CD90 in the fluorescence analysis—those were likely CD63-only vesicles that were captured but not tagged with a fluorophore. Despite this limitation, the reports clearly demonstrate CD63 positivity via the high particle counts on CD63 capture spots. Every sample's CD63 bar was tall, confirming that CD63 is a dominant marker on these exosomes (FIG. 10).

c. Co-Expression:

Many CD63-positive exosomes also had CD9 or CD81 (some vesicles likely carry all three tetraspanins). Indeed, if an exosome had CD63 and CD9 or CD81, it might be captured on either type of spot. The co-expression could not be explicitly quantified via fluorescence for CD63, but logically there is overlap. The evidence for this is indirect: for example, some exosomes captured on CD9 were probably CD63-positive as well (though CD63 wasn't labeled, their presence on CD9 spots means they at least had CD9). Conversely, exosomes on CD63 spots could also have CD9/CD81 (and those would have shown up as CD9 or CD81 positive in detection). In the study, a different anti-CD63 detection antibody was tried without success, so no direct numeric co-expression with CD63 could be measured. Nonetheless, CD63 is clearly abundantly expressed, and likely co-exists with the other tetraspanins on many of the exosomes (given all three are common exosome proteins). CD63 is a major marker on these equine exosomes, even though it was measured only by capture.

In summary, CD63 had the highest exosome capture, meaning most exosomes in these samples are CD63-positive—a fact confirmed by strong CD63 capture signals in the NanoView reports, despite the lack of fluorescent staining for CD63.

### 4. CD90 (Thy-1, an MSC Marker)

a. Particle Counts & Percentage:

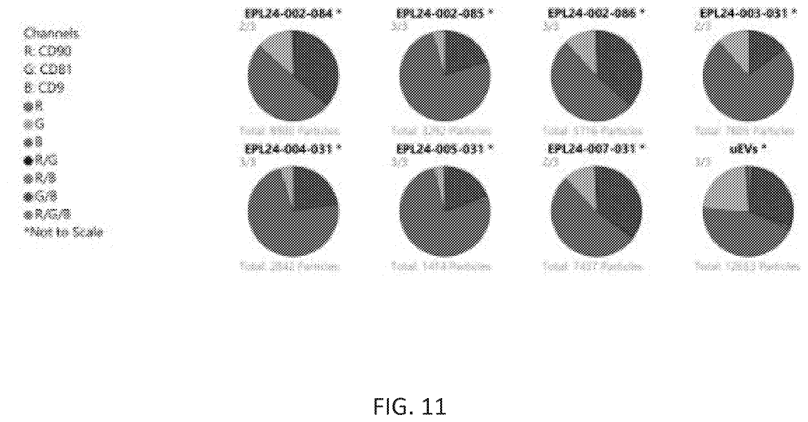

CD90, also known as Thy-1, is a surface marker typically found on mesenchymal stem cells. It was investigated whether a subset of the exosomes carry CD90. The NanoView analysis used a fluorescent anti-CD90 antibody (labeled AF647) to detect this marker on captured exosomes. The results showed that only a small fraction of exosomes were CD90-positive. Quantitatively, approximately 5% of the exosome particles were positive for CD90 across the samples. In practical terms, this means that for every 100 exosomes, only about five carried the CD90 protein—a relatively low representation. The NanoView data's bar graphs and pie charts (FIGS. 10 and 11) reflect this low percentage: the CD90 detection signal was modest, barely above background in many samples, especially compared to the strong signals from tetraspanins. An isotype control experiment (using a non-specific antibody) showed minimal binding, which helps confirm that the small CD90 signal is real and not an artifact.

b. Co-Expression:

Importantly, those few CD90+ exosomes almost always also carried one of the tetraspanin markers (CD9 or CD81). A population of CD90-only exosomes with no other markers was not observed—indeed, that would be unlikely, since the exosome needed to be captured via CD9/CD63/CD81 or a general surface to be detected at all. The study included double-staining tests for CD9-CD90 and CD81-CD90 co-expression, demonstrating that exosomes could be positive for both an exosome marker and CD90. In the NanoView co-localization analysis, CD90 signals overlapped with CD9/CD81 signals: e.g. some exosomes captured on CD9 or CD81 spots lit up with the CD90 antibody, indicating CD90-CD9 double-positive vesicles. The report's co-expression chart indeed listed combinations like “CD90-CD9” and “CD90-CD81,” which confirms these overlaps were observed. However, no exosome was found to be CD90-positive without also being positive for at least one of the exosomal tetraspanins, as expected.

Visualization-wise, the NanoView report for CD90 included a small pie chart (or equivalent summary) showing CD90⁺ vs CD90⁻ particles, where the CD90⁺ slice was only a tiny sliver (~5%). Additionally, a bar in the co-expression heatmap corresponding to the intersection of CD90 and CD9 was present but very short, underscoring the rarity of CD90-bearing exosomes (FIG. 12). In summary, CD90 was detected on a minor subset (~5%) of exosomes, and those CD90+ vesicles were generally also carrying standard exosome markers (CD9/CD81). This finding is consistent with CD90 being a cell-specific protein that only a small portion of exosomes incorporate.

### 5. CD105 (Endoglin, an MSC Marker)

a. Particle Counts & Percentage:

CD105 is another mesenchymal cell marker (endoglin) that was probed in the exosome samples. Like CD90, CD105 was evaluated by fluorescent antibody staining in a separate NanoView run (with an AF647-labeled anti-CD105 detection antibody). The results mirror the CD90 findings: only a very small percentage of exosomes were CD105-positive. In fact, CD105-positive particles were as rare as—or even slightly rarer than—the CD90-positive ones. It was observed on the order of a few percent of exosomes carrying CD105. For example, in the second batch of samples (8-13), the CD105 signal was barely above background in most cases. Overall, roughly ~2-5% of exosomes had CD105 on their surface (the exact percentage is low and may vary per sample). This aligns with expectations, as not all exosomes from these cells would include CD105. The NanoView report's detection charts for CD105 showed minimal counts, confirming it is an uncommon marker on the vesicles. As a visual confirmation, an experiment was done with exosomes incubated with the CD105 antibody versus controls; the CD105-stained exosomes showed only a slight increase over the PBS (buffer) control, indicating how small the CD105\hat{ }+ subset is.

b. Co-Expression:

Similar to CD90, any CD105+ exosomes were also positive for exosomal markers like CD9 or CD81. There was no evidence of exosomes bearing CD105 alone. The assay design required exosomes to be captured on one of the tetraspanin antibody spots (or a general EV spot) before detection, so a CD105-bearing exosome would almost certainly also have at least one tetraspanin to enable capture. Indeed, the co-expression analysis indicates that CD105 frequently co-occurred with CD9/CD81 on the rare vesicles where it was present. For instance, some exosomes on CD9 or CD81 capture spots showed a CD105 fluorescent signal, implying CD9-CD105 or CD81-CD105 double-positive exosomes. The report for samples 8-13 included a co-localization analysis (for capture CD9 and CD81) that would flag any CD105 overlap—while the text of the report primarily highlighted CD9/CD81 overlaps, those few CD105-positive vesicles are inherently counted in the “any marker” category. Overall, CD105-positive exosomes were very scarce (a few percent at most) and only found as part of exosomes that also had the usual exosome markers.

It's worth noting for clarity: CD90 and CD105 are markers of the parent cells (mesenchymal cells), and seeing them on exosomes indicates that a small subset of exosomes carries cargo from the cell membrane. The low percentages (on the order of 5% or less) of CD90+ or CD105+ exosomes are therefore not surprising. Most exosomes primarily exhibit the universal exosome markers (CD9, CD63, CD81), with only a minority displaying these specialized proteins.

### 4. Discussion

The analysis of the NanoView exosome reports demonstrates that: (1) CD9, CD81, and CD63 are strongly present on equine umbilical cord samples (with CD63 being the most widespread, and many exosomes carrying at least one of CD9 or CD81); (2) CD90 and CD105 are present only on a small minority of exosomes (~5% or fewer); and (3) markers often co-exist on the same vesicles (e.g. an exosome that has CD90 will almost certainly have CD9 or CD81 as well). The combination heatmaps/overlap analyses from the reports confirm these co-expression patterns, such as significant CD9-CD81 overlap and minor populations of CD90-tetraspanin overlap. The key takeaway is that most exosomes carry the common exosomal markers (CD9/CD63/CD81), while only a few carry the specialized MSC markers CD90 or CD105. The NanoView system's charts (bar graphs of particle counts and pie charts of marker percentages) visually reinforce these points by showing big sections for tetraspanins and very small sections for CD90/CD105 positivity. Overall, the data shows that the equine exosome samples are rich in classical exosome markers, and only a small subset displays MSC-specific markers.

### Required EV Markers: Tetraspanin and Cytosolic Protein

The International Society for Extracellular Vesicles (ISEV) recommends demonstrating the presence of at least one membrane-bound (Category 1) marker and one cytosolic (Category 2) marker in any extracellular vesicle (EV) preparation. These two categories of markers serve to confirm the vesicular nature of the sample: Transmembrane EV Marker (Category 1): A membrane protein (often a tetraspanin like CD9, CD63, or CD81) should be detected to indicate the presence of the vesicle's lipid bilayer membrane. Tetraspanins are hallmarks of EV membranes and their presence demonstrates that the particles have the characteristic vesicular membrane structure. Cytosolic EV Marker (Category 2): An internal (intravesicular) protein should be detected to show that the vesicles contain enclosed intracellular material. Common examples are cytosolic proteins with membrane-binding capacity (e.g., ALIX or TSG101); finding such a protein confirms that the preparation indeed has membranous vesicles carrying intracellular contents.

Detecting markers from both categories is crucial for confidently identifying the isolate as EVs. The combination of an external membrane marker and an internal luminal marker fulfills the minimal criteria to “demonstrate the EV nature” of the sample per ISEV's guidelines.

### Proteomic Evidence of Cytosolic Marker (ALIX) in the EV Sample

In the study, initial analyses did not capture cytosolic proteins (likely due to the preparation or isolation methods focusing on surface markers). However, subsequent proteomic analysis of the EV samples provided the needed evidence for a cytosolic marker. Notably, we identified Programmed Cell Death 6-Interacting Protein (PDCD6IP)—the protein commonly known as ALIX (also referred to as AIP1)ncbi.nlm.nih.gov—in high abundance in the EV proteome. ALIX/PDCD6IP is a well-known cytosolic EV marker associated with the endosomal sorting complex required for transport (ESCRT) pathway, and is routinely used to verify the presence of exosomes and other small EVs.

The detection of ALIX (PDCD6IP) in the EV preparation thus satisfies the Category 2 (cytosolic protein) requirement from the ISEV guidelines. Coupled with the presence of a tetraspanin membrane marker in the samples (fulfilling the Category 1 requirement), there is evidence for both an EV surface marker and an intravesicular marker. Together, these findings confirm that the isolated particles are indeed bona fide EVs, in line with the ISEV's recommended criteria for EV characterization. This comprehensive marker analysis strengthens the conclusion that the sample's contents are enriched in extracellular vesicles rather than contaminants.

### 5. Conclusion

Everything measured lines up with the defining features of exosomes, a specific subclass of small EVs: Size (40-160 nm): NTA confirmed the majority of particles fall squarely in the 50-140 nm range, typical of exosomes. Membrane markers (tetraspanins CD9, CD63, CD81): NanoView demonstrated that each sample carries one or more of the canonical exosome tetraspanins on its surface, with CD63 being the most prevalent and CD9/CD81 co-expressed on a sizable fraction of vesicles. Cytosolic marker (ALIX/PDCD6IP): mass-spectrometry data showed strong abundance of ALIX (PDCD6IP), an internal ESCRT-associated protein routinely used to validate exosomes.

According to the MISEV2018 guidelines, confirming at least one membrane-bound and one cytosolic EV marker is sufficient to identify exosomes within a broader EV population. Because the samples meet both criteria, one can confidently conclude that the particles in the Equicenta™ CTM preparations are indeed exosomes, rather than other non-exosomal vesicles or contaminants.

### F. Example 6—Equine Umbilical Tissue Suspension (equicenta™ CTM) Demonstrates Greater Effectiveness and Biocompatibility Compared to Standard of Care in Lame Performance Horses: A Prospective, Multi-Center, Controlled, Randomized Clinical Study

Umbilical tissue microparticles, specifically Wharton's jelly, have been safely used in humans for joint, tendon, and ligament applications. Cryopreserved equine umbilical tissue suspension (equicenta™ CTM; equicenta™ CTM) has been shown to consistently contain over 2600 proteins, many of which are anabolic, anti-catabolic, anti-inflammatory, and fibrillar, and to be biocompatible in normal horses. This multi-center, prospective, randomized, controlled clinical trial (AVMA VCT26005934 and IACUC-ETCR-24-0329) was designed to determine if equicenta™ CTM was safe and effective in lame performance horses compared to non-steroidal standard of care (SOC), primarily biologics. One hundred thirty-eight (138) client-owned lame horses with 195 conditions of osteoarthritis, desmitis, and/or tendonitis were followed for 26 weeks. Eight clinical sites (11 veterinarians) enrolled performance horses with confirmed diagnoses and client consent. Horses meeting criteria such as AAEP grade 1-4 lameness and a 30-day washout period from other treatments and procedures, were randomly assigned to receive a single dose (1.5 mL) of equicenta™ CTM or SOC on day 0 and followed at 2, 4, 12, and 26 weeks, with >98% compliance. Ordinal data for lameness, pain, swelling, and performance was analyzed by 2-factor repeated measures ANOVA. Significance was set at p<0.05. Horses receiving equicenta™ CTM showed greater improvement in lameness, pain, swelling, and performance over time compared to SOC-horses, and a greater number of equicenta™ CTM-horses returned to training by 6 months (p<0.01). Quantitative ultrasound analysis demonstrated greater improvement from baseline in equicenta™ CTM-ligaments, while SOC-ligaments showed a lower percent response (p <0.02). Control SOC-horses had greater odds of adverse events potentially related to the product than equicenta™ CTM-horses (Odds Ratio SOC:equicenta™ CTM 4:1; p=0.05). equicenta™ CTM offers additional advantages such as rapid thaw for same-day use, no on-site processing, and consistent composition. These positive outcomes support the clinical use of equicenta™ CTM in sport horses and provide veterinarians with a high level of evidence to support this recommendation to clients.

### 1. Introduction

Allografts have been used for decades to repair injuries in joints, tendons, and bones, with few reported issues. Umbilical tissue in humans has been well-studied and applied in musculoskeletal conditions as a flowable microparticulate composed of loose-connective tissue matrix, known as Wharton's jelly, excluding the umbilical vessels or amnion. This connective tissue matrix contains soluble and fibrillar proteins that supplement structure and trophic processes. In humans, Wharton's jelly has been applied in over 100 sites with significant improvements in joint, ligament, and tendon pain and function without adverse events, possibly due to the immune privilege of fetal tissue. Prospective clinical trials are in progress.

Equine microparticulate tissue allograft uses for managing equine musculoskeletal conditions has been implemented in the United States over the last decade, including dental pulp and amnion. Dental pulp tissue, harvested from unerupted tooth buds in stillborn foals and processed into microparticles, demonstrated significant short-term (two-week) improvement in lameness and pain compared to saline control in horses with osteoarthritis or desmitis; however, that product is no longer commercially available. Several equine amnion products have been commercialized, but peer-reviewed evidence of characterization, safety, or efficacy for the current formulations on the market is limited or absent.

Prior published work has characterized and confirmed the biocompatibility of a novel cryopreserved, minimally processed native particulate umbilical tissue allograft suspension (equicenta™ CTM), stored at −80 °C and thawed just prior to use, in joints and ligaments in normal horses and a limited number of diseased horses. Equicenta™CTM is a uniquely selected portion of umbilical tissue, single-sourced, obtained, and stored using quality practices that align with Food and Drug Administration guidelines for Human Cellular and Tissue-Based Products10. Variability among and within batches is low (4.6%). Cytokine analyses further confirmed the lack or very low content of inflammatory cytokines such as IL-1, -2, -4, -8, and -15 in the suspension.

The goal of this study was to target real-world painful conditions of osteoarthritis (OA), tendonitis, or desmitis (soft tissue injury) in a prospective, multi-center, randomized, controlled clinical trial design under defined environments and standards, limited to eight performance horse practices and eleven protocol- and consensus-trained veterinarians. equicenta™ CTM umbilical tissue, used as an injectable for painful soft tissue injury (desmitis or tendinitis) or OA, can affect events associated with tissue injury (pain and inflammation) and result in greater improvements in outcomes, including lameness, in performance horses.

### 2. Materials and Methods

This prospective, approved, controlled clinical trial (AVMA VCT26005934 and IACUC-ETCR-24-0329) compared an intervention (Equicenta™ CTM) to an open-label, contemporaneous, parallel standard of care (SOC; non-steroidal), which included other commercially available biologic therapies. Performance horses meeting the inclusion criteria appendicular lameness (AAEP grade 1-4), a confirmed diagnosis of osteoarthritis, desmitis, or tendonitis, and client consent-were enrolled. Subjects were assigned to treatment groups using a modified alternating block design. Initial random assignment was performed via coin toss at each practice site, followed by alternating assignments in a prospective serial manner, with limited exceptions to achieve an estimated 60:40 (1.65:1) allocation of equicenta™ CTM to SOC. Affected conditions were aseptically injected with a single 1.5 mL dose of equicenta™ CTM or the biologic/physical therapy plan considered best for the horse's condition (control). Primary outcomes assessed through 26 weeks included safety (adverse events and ordinal scores for lameness [0-5], pain [0-4], and swelling [0-4] post-injection) and effectiveness (lameness improvement and performance outcomes). Clients had the option for a second injection of control or equicenta™ CTM if lameness was the same as baseline at 90 days or at the end of the study if the horse was not sound. Safety information was collected for at least 30 days following second injections. Prospective client assessment surveys were designed to collect relevant safety and comfort data, such as comfort at turnout or appetite, not obtainable by the veterinarian. Study veterinarians completed enrollment, day 0, and follow-up examinations at 2, 4, and 12 weeks post-injection. Week 26 follow-up information, including performance data and a final survey, was obtained by a study veterinarian in consultation with the client.

Ultrasound series from forty-two horses (46 entries; 34 ligaments, 12 tendons) met quality criteria and power for analysis in a randomized, blinded manner. Thirty horses received umbilical allograft (equicenta™ CTM), while sixteen were SOC controls. Approximately 1,400 regions of interest (ROIs) were manually delineated.

All statistical analyses were performed using commercially available software.

Frequency data (counts of scores) were analyzed to compare groups using Fisher's Exact Test and Odds Ratios (demographic data, adverse events). A two-factor repeated measures analysis of variance was performed for group and condition over time for lameness, pain, swelling, and performance. Additionally, to normalize the data, values were compared to baseline within each group, and data were displayed cumulatively to reflect the status of lameness, pain, swelling and performance as horses progressed through the study. Client survey scores (7 categories; 10-point Likert scales) were expressed as boxplots and compared by one-way ANOVA across time per category between equicenta™ CTM and SOC. Differences were considered statistically significant if p<0.05.

### 3. Results

One hundred and thirty-eight (138) horses with 195 conditions and 224 injections completed the trial. The distribution of conditions was osteoarthritis (OA; n=104, 53%) or soft tissue (ST) injury (n=91, 47%), including suspensory desmitis (n=63, 69%) and tendonitis (n=28, 31%). Veterinarians and clients completed >98% of follow-up surveys, with 100% completion at day 0, 12, and 26 weeks. The equicenta™ CTM and SOC populations did not differ significantly in body weight, sex, breed, affected limb, condition duration, or frequency of prior treatment. Horses represented various disciplines, including competitive dressage (13.8%), eventers, hunters and jumpers (21%), racing (36.2%; flat, sulky, barrel), western performance (21.7%; reining, cutting, rodeo), other (2.4%; driving, gaited show horses) The most represented breeds were: Thoroughbreds (55), warmblood sport horses (34), and Quarter Horses (34).

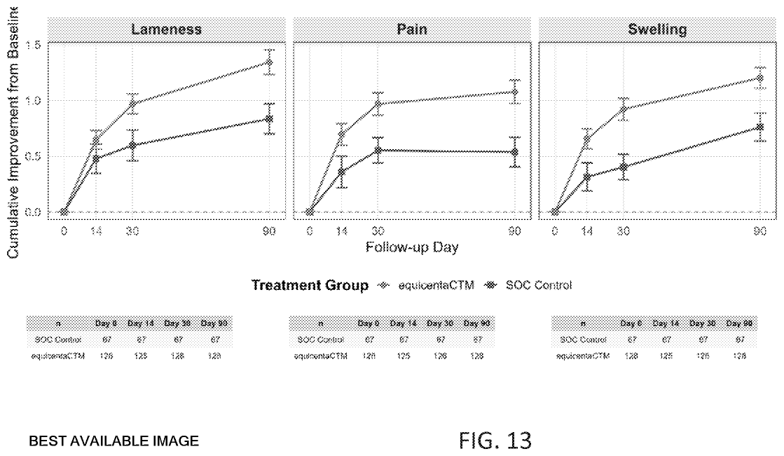

Horses receiving equicenta™ CTM showed significantly greater improvement in lameness, pain, swelling and performance scores over time (cumulative improvement) compared to SOC horses, starting at day 30 for lameness and day 14 for pain and swelling (p<0.01). The greatest significant differences were in the cumulative improvement in pain over time and in joints (P<0.01). Swelling scores of equicenta™ CTM horses showed significantly greater cumulative improvement over time compared to SOC horses (p<0.05). (FIG. 13).

Ligaments treated with equicenta™ CTM demonstrated significant ultrasound improvement from baseline (mean 53.8±7.2; p=0.019), while SOC controls declined between 1 and 3 months (44.0±6.5; p=0.012), yielding a large between-group effect (Cohen's d=0.98; Mann Whitney p=0.013). Nine of 23 equicenta™ CTM-ligaments (39%) reached the responder threshold (≥55) versus 0/11 SOC-ligaments (Fisher's p=0.015). The late response trajectory occurred exclusively in equicenta™ CTM-horses (20% vs. 0%; p=0.047). Four horses demonstrated clinical improvement but had a score of <45, indicating return-to-work risk. The score at day 14 correlated with final score (r=0.88).

SOC horses had a significantly greater number of adverse events potentially related to the product than equicenta™ CTM horses. Specifically, the odds ratio for an adverse event potentially related to the product for SOC compared to equicenta™ CTM was 4:1 (p=0.05), and these events occurred early after injection.

### 4. Discussion

This study is the first to report the use of umbilical-derived tissue suspension, specifically Equicenta™ CTM with proprietary processing (patent pending), in client-owned performance horses for musculoskeletal conditions causing lameness. This study, in combination with the previously published peer-reviewed safety and characterization study, provides veterinarians with a robust tool kit to support the use of e equicenta™ CTM in lame client-owned horses. In addition to demonstrating effectiveness and biocompatibility in real-world musculoskeletal conditions, other advantages include off-the-shelf access with rapid thawing (~10 min from the −80 °C freezer) for same-day application. A single injection offers the advantage of reduced stress for horses compared to hauling and repeated injections often recommended with autologous plasma and serum products, as was performed for autologous conditioned serum and plasma alpha 2 M in this study. The efficiency of a single-dose application saves veterinarian and technician time and expense. Furthermore, a single injection reduces the risk of documented concerns, including post-injection infection, adverse reactions, and a greater number of needle tracts in dense soft tissues such as ligaments and tendons.

Autologous hemoderivative products have been in clinical use in horses for more than a decade but are reported to have variable characterization and clinical results. Autologous platelet, plasma, and serum products will always be limited by donor variability (often an adult or aged horse with co-morbidities), equipment variability in maintenance and/or function, and inflammatory systemic co-morbidities such as osteoarthritis, chronic pain, or metabolic derangements. Fetal tissue from vigorous, healthy, growing foals, quality processed from a single source, provides a consistent, potent source of anabolic and anti-catabolic proteins in a native scaffold microparticle.

The application of this intervention was purposefully targeted for delivery to the local site of tissue injury. The soluble proteins, in combination with a fetal tissue scaffold, appear best applied directly into defects in tendons and ligaments as a connective tissue to connective tissue transfer. In joints, veterinarians were instructed to directly inject equicenta™ CTM near the site of suspected or known cartilage injury. The goal was to deliver the protein solution and microparticles to the synovium close to the injured cartilage to target the site of the most inflamed synovium and deliver anabolic and anti-catabolic proteins in the vicinity of the cartilage. The greatest improvement in lameness and pain was observed in joints, an unanticipated finding. The standard of care (SOC) joints, on average, declined back toward baseline values at 12 weeks, diverging from the equicenta™ CTM joints, which continued to improve.

Many commercial biologic products are on the equine market, but few have provided the equine industry community with evidence of efficacy in vivo. In vitro studies are abundant and too numerous to report here. In vivo briefs on websites are often of low evidence level, such as testimonials, case series, or retrospective studies. Two higher-level, controlled studies have reported efficacy in vivo. In one, autologous conditioned serum used an experimental chip fracture model of OA with sham and saline controls, and in one, autologous protein solution used clinical cases with saline controls in OA. The limitations of experimental models include limited transference to real-world clinical scenarios. The limitation of the prospective clinical study was the short duration of two weeks. One case series of amnion product in lame horses with desmitis used retrospective controls relying on questionnaire follow-up with a low response rate. Prospective controlled studies in real-world cases are logistically challenging and benefit from a dedicated team of veterinarians and clients and a multi-center director, particularly for longer-term studies such as six months.

In summary, in a prospective interventional model with contemporaneous comparator controls (including other injectable biologics), equicenta™ CTM demonstrated greater safety (adverse events/lameness/pain/swelling post-injection) and effectiveness (improved lameness, pain, swelling and performance outcomes) than the standard of care out to 26 weeks in real-world lame performance horses. Equicenta™ CTM was non-inferior in parameters that did not differ and offers other advantages, such as rapid thaw for same-day use and consistent composition.

### G. Example 7

Equicenta™CTM is a cryopreserved micronized equine umbilical tissue allograft that is known to contain an abundance of fetal proteins in a native umbilical connective tissue matrix CTM). This Example looked to microscopically visualize the presence of scaffold networks in the tissue particles and confirm their biodegradation potential and associated protein release.

The objectives of these studies were to microscopically: 1) visualize and evaluate in vitro, two bio-enzyme digestion methods to confirm potential for biological degradation of equicenta™CTM in vivo and subsequent release of proteins relevant to healing; and 2) visualize fresh-thawed cyto-spin smears of microparticles for structure and contents.

### 1. Methods

Microscopic characterization of equicenta™CTM. equicenta™CTM was thawed at 23 °C, pelleted by microcentrifugation, applied to glass slides and stained with hematoxylin for microscopic assessment. Equicenta™CTM was cultured in T-25 flasks with media+FCS, or after 1 hour of collagenase I exposure at 37 °C (Collagenase+equicenta™CTM) or after 30 minutes in 0.25% trypsin at 37 °C (Trypsin+equicenta™CTM). Digestion of equicenta™CTM for 0.5, 2 and 24 hours was performed with saline as a negative control, trypsin as a positive control and collagenase I, as a biological enzyme. Protein concentrations were determined via Bradford assay and microscopic images were obtained at each time point.

Biological characterization of equicenta™CTM. (1a) equicenta™CTM was extracted with Triton-X and (2a) evaluated for cytokine and growth factor concentrations using ELISA techniques. Presence of extracellular vesicles (EVs)/exosomes was determined via (1b) isolation through an ultracentrifugation process followed by (2b) NanoView Exoview® Platform analysis for tetraspanin markers CD9, CD81, and CD63 as well as the presence of cytosolic ALIX through mass-spectrophotometry.

### 2. Results

FIG. 9A shows cytological evaluation of pelleted equicenta™CTM fixed with cold methanol:acetone and stained with hematoxylin. Note the presence of filamentous-form matrix (purple arrows) observed with pink background staining indicating a proteinaceous extracellular matrix creating a net-like protein scaffold structure. Bar (A)=200 um,

FIG. 9B shows microscopic evaluation of umbilical tissue allograft (equicenta™CTM) in culture (top) alone (equicenta™CTM alone), (middle) equicenta™CTM after collagenase I digestion and (bottom) equicenta™CTM after trypsin digestion. In culture media, equicenta™CTM had aggregates of extracellular matrix that did not attach to the plastic and floated within the fluid as would be anticipated for matrix particles. After enzymatic digestion with collagenase I, the aggregates became smaller and fewer over 2 weeks, indicating digestion of collagens and loss of particulate form. As this enzyme is present in naturally occurring tissue, these findings indicated that equicenta™CTM would undergo a natural biodegradation with enzymes found in vivo, particularly in injured tissue. Trypsin digestion of particles was rapid and complete compared to control equicenta™CTM and collagenase digested cultures. Bar=200 um.

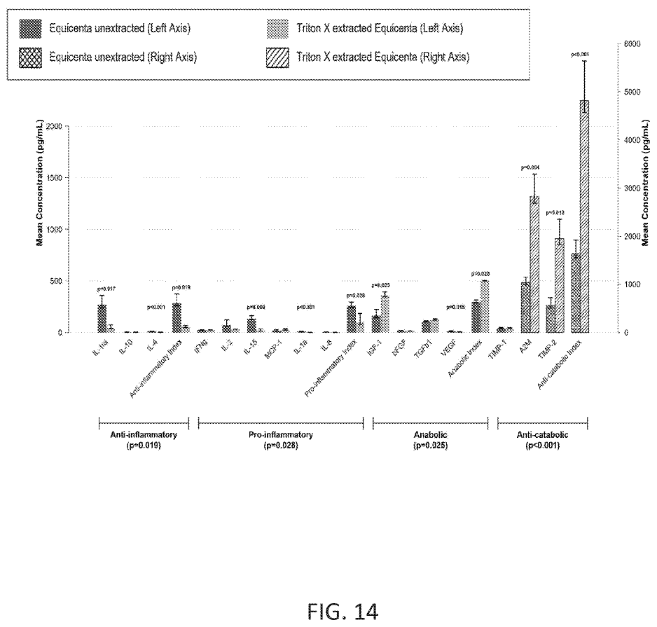

FIG. 14 shows growth-factor release over time. The data demonstrates the allograft acting as a reservoir with time-dependent release. FIG. 14 shows cytokine and growth factor concentrations of equicenta™CTM before (dark bars) and after (light bars) Triton-X extraction. Note the significant decrease in inflammatory indices and significant increase in anabolic and anti-catabolic indices, specifically marked increase in IGF-1, A2M, and TIMP-2.

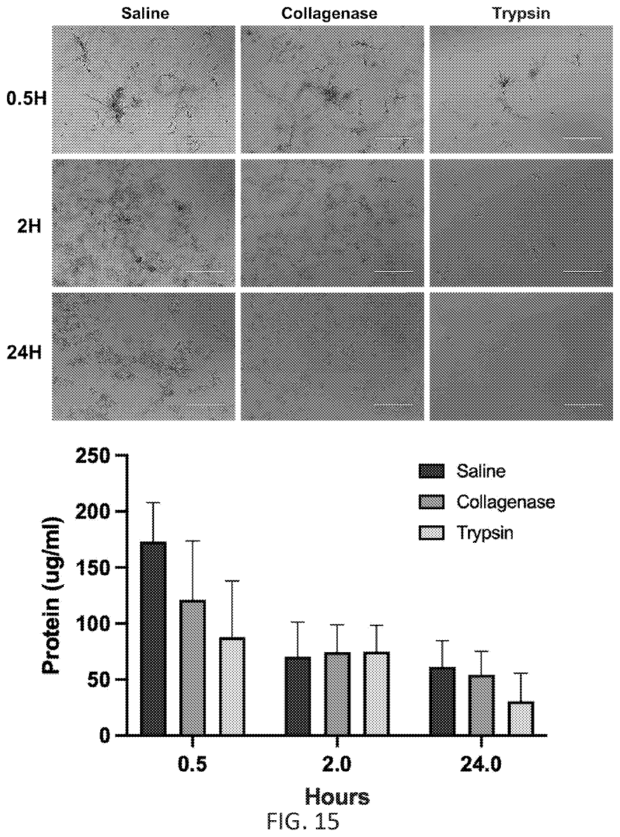

FIG. 15 shows microscopy and protein concentration of equicenta™CTM in saline or after digestion with collagenase I or trypsin for 0, 0.5, 2, and/or 24 hours. Note the equicenta™CTM protein degraded under all conditions over time. Microscopic images showed loss of the net like structure and smaller particle size noted with collagenase digestion. Trypsin (positive control) rapidly and completely degraded the structure.

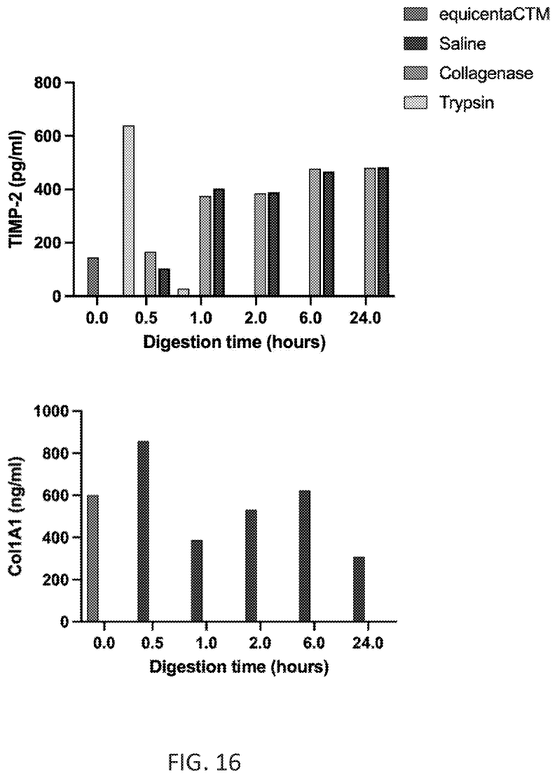

FIG. 16 shows TIMP and Col1A1 concentrations in a sample of equicenta™CTM in saline or digested with collagenase I or trypsin. Note the increase of TIMP-2 over time in the saline and collagenase digestion. Trypsin exposure (as a positive control) resulted in quick release of TIMP-2 at 0.5 h and complete degradation with longer exposure. While natural release of TIMP-2 from the product occurred when unsupported in saline alone or when the matrix was degraded with collagenase. Evidence of collagen degradation was observed with collagenase and trypsin at 0.5 h followed by slower release from saline alone.

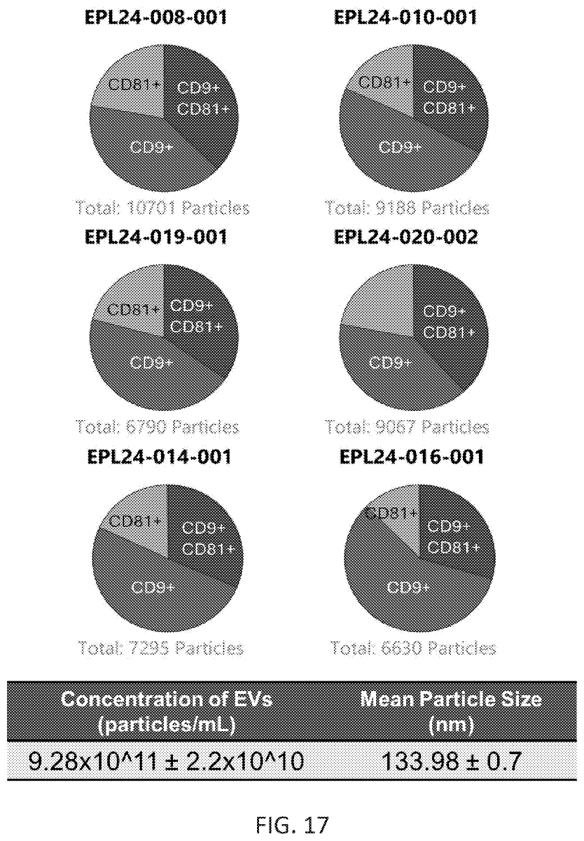

FIG. 17 shows isolation of extracellular vesicles (EVs), also referred to as exosomes, from different donors of equicenta™CTM and analysis for marker expression. Nanoparticle tracking analysis found that CD9 capture bound 1.4×10\hat{ }4-1.3×10\hat{ }7 particles/ml with 15-35.9% of these particles also positive for CD81. Additionally, a small fraction of these particles also expressed the mesenchymal stem cell (MSC) marker CD90. CD63 capture-bound particles were also able to capture CD9 (53-83%). In addition to the tetraspanins from the cell surface, the cytosolic marker ALIX (PDCD61P) was found in strong abundance via mass-spectrometry. In conclusion, size and markers confirmed billions of EVs were present in equicenta™ CTM.

### 3. Conclusions

Equicenta™CTM has a fibrillar matrix structure that is maintained in culture media, a similar composition to normal biological environment.

Equicenta™CTM serves as a reservoir for anabolic and anti-catabolic proteins that are released over time, supporting potential for sustained release in vivo and support of the healing process over time.

Equicenta™CTM when thawed and directly administered can provide a fetal protein scaffold for endogenous cell migration and tissue healing. Biodegradation using natural-occurring proteolytic enzymes found in the CTM of injured joints, tendons, and ligaments, further released fetal matrix proteins locally, such as IGF, A2M, and TIMP-2 that likely augment the healing process.

### H. Example 8—Exposure of Articular Tissue to Cryopreserved Equine Umbilical-Derived Allograft (Equicenta™CTM) in an Ex Vivo Explant Model of Arthritis Supported Tissue Compatibility

### 1. Introduction

Osteoarthritis is the most common cause of lameness in horses and has recently been reported as the most common medical condition in senior horses (≥15 years of age) that are still competing in the US. The pathophysiology of osteoarthritis is relatively well understood and is comprised of acute or repetitive trauma, followed by joint inflammation and subsequent cartilage degradation. The inflammatory mediator interleukin-1β (IL-1β) plays a critical role in the development of osteoarthritis. IL-1β is markedly increased in chondrocytes and synoviocytes from patients with osteoarthritis, and a correlation between synovial fluid IL-1β concentrations and severity of cartilage damage has been reported in patients with knee trauma. Furthermore, chondrocytes are the cellular target of IL-1β, with high expression of the interleukin-1 receptor (IL-1R) that is increased in patients with osteoarthritis. The binding of IL-1 to its receptor increases cartilage catabolism by inducing the production of proteolytic enzymes, such as matrix metalloproteinases, which are controlled by tissue inhibitors of metalloproteinases (TIMPs). The uncontrolled production of matrix metalloproteinases during osteoarthritis development subsequently degrades collagen and aggrecan, thereby causing cartilage damage.

A variety of treatments are used to manage osteoarthritis in horses, including systemic and local therapies, with varying degrees of reported success. New therapies are continually sought to improve outcomes and eliminate adverse effects of pharmaceuticals, such as corticosteroids, which have been commonly administered intra-articularly in horses with osteoarthritis for many years. Orthobiologics are being increasingly applied in human and equine musculoskeletal disorders, including traumatic joint injuries and arthritis. Tissue allografts represent a promising biologic option, but rigorous evaluation is essential to establish evidence-based guidelines for clinical use. Numerous biologic products are marketed for equine joints to address cartilage injury and synovitis, often via intra-articular injection to relieve pain, reduce inflammation, and slow degeneration. While some claim regenerative potential, supporting evidence remains limited.

The novel orthobiologic product, equicenta™CTM, was recently developed from equine umbilical cord tissue for the management of joint disease in horses. The umbilical cord is a fetal tissue rich in Wharton's Jelly, a gelatinous connective matrix surrounding the umbilical vessels. Wharton's Jelly contains hyaluronic acid, growth factors, glycosaminoglycans, glycoproteins, and mesenchymal stem cells within a collagenous framework. Its fetal origin confers immune tolerance, and through proprietary processing, these primordial tissues can be prepared with minimal manipulation for homologous use. The umbilical-derived microparticles in equicenta™CTM have been reported to be safe and biocompatible for intra-articular use in horses. This product has been found to contain thousands of fetal proteins, many of which exhibit properties that support tissue healing.

Ex vivo explant models have been used to study the effects of mediators implicated in the development of arthritis and to evaluate potential therapeutics. This study aimed to determine the effects of cryopreserved equicenta™CTM on cartilage and synovial explants stimulated with and without recombinant equine IL-1β (re IL-1β), a catabolic cytokine central to the development of osteoarthritis. Equicenta™CTM can be histologically compatible with articular tissue as well as limit tissue inflammatory effects and cartilage degradation in this ex vivo model of osteoarthritis, assessed by histomorphometry, prostaglandin E2 (PGE2) and soluble glycosaminoglycan (sGAG) release.

### 2. Methods:

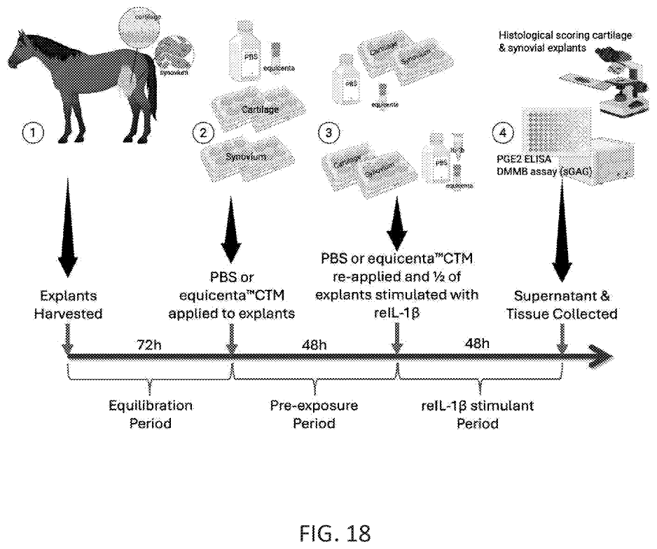

a. Explant Collection and Stimulation (FIG. 18):

Cartilage and synovial membrane were aseptically collected from clinically normal femoropatellar joints of three horses euthanized for reasons unrelated to the study. Full-thickness cartilage and synovium were cut into sections (6×6×1 mm for cartilage and approximately 100 mg per synovial segment). Explants from each horse (n=12 explants for each tissue per horse) were placed into twelve wells of 6-well plates with media comprised of Dulbecco's modified eagle medium (DMEM), 10% fetal calf serum (FCS), penicillin and streptomycin (100 U/ml), and cultured at 37 °C, 5% CO2 for 72 hours to allow the tissues to equilibrate to the ex vivo environment. Following equilibration, twelve cartilage and twelve synovial explants from each horse were then pre-exposed to a 1:5 dilution of either phosphate-buffered saline (PBS; n=6 per horse as a negative control) or a cryopreserved umbilical-derived matrix product (equicenta™CTM, n=6 per horse) in DME medium and incubated for 48 hours. The concentration of PBS or equicenta™CTM at a 1:5 dilution in media was used as an estimate of the concentration that may be present in a clinically treated joint corresponding to a dose of 1.5 into 7.5 mL of synovial fluid. Equicenta™CTM is a commercially available, minimally manipulated umbilical-derived tissue allograft, available as microparticles suspended in solution and cryopreserved. The umbilical-derived tissue allograft is ethically obtained following full-term, natural delivery, ensuring no harm comes to the mare or foal, and is sourced from a closed herd. The product is thawed at room temperature just prior to use in this study and application into the respective treatment wells. After 48 hours of pre-exposure, the PBS or equicenta™CTM was removed, the explants were washed, and one of the four conditions listed below was applied in triplicate:
1. Media+PBS (negative control; n=3 per horse)
2. Media+equicenta™CTM (equicenta™CTM control; n=3 per horse)
3. Media+PBS+stimulation with recombinant equine IL-1β (reIL-1β; 10 ng/ml) (reIL-1β control; n=3 per horse)
4. Media+equicenta™CTM+stimulation with reIL-1β (10 ng/ml; n=3 per horse)

Explants that were pre-exposed to PBS remained exposed to PBS, and those exposed to equicenta™CTM remained exposed to equicenta™CTM. After reapplication of the exposure treatment ±the stimulant reIL-1β, all explants were incubated for an additional 48 hours at 37 °C, 5% CO2, after which the supernatant was collected and stored at −80 °C. Explant tissue was placed in 10% formalin and processed for later histological evaluation.

b. PGE2 ELISA:

Supernatant from the cartilage and synovial explants was analyzed in duplicate using a commercially available kit (R&D Systems; Minneapolis, MN). Supernatant from cartilage was diluted with assay diluent at a ratio of 1:3 and 1:10 for cartilage supernatant and at a ratio of 1:25 and 1:75 for synovial supernatant. Briefly, primary antibody was added to each sample and incubated for one hour, followed by the addition of PGE2 conjugate. After washing, substrate and stop solutions were added to each well. OD values were determined for each sample using a spectrophotometer (Biotek Synergy, Agilent, Santa Clara, CA) and were compared to the standard curve using 4-parameter logistic regression. Mean PGE2 concentrations for each sample were determined and used for statistical analysis.

1. sGAG concentration:

Sulfated GAG released into the explant media was evaluated in the supernatant from all wells of both the cartilage and synovial explants using the previously described Dimethylmethylene Blue Assay (DMMB). Supernatant from each explant sample was assessed in triplicate. Supernatants from cartilage samples were diluted 1:10 with media for analysis. Supernatants from synovial samples were not diluted. Briefly, 50 ul of sample was added to 200 ul of DMMB reagent (Millipore Sigma; Burlington, MA) at a pH=1.5 in each well of 96-well plate. The absorbance was immediately determined using a spectrophotometer (Biotek Synergy, Agilent; Santa Clara, CA) at 525 and 590 nm after 5 seconds of shaking. Sample concentrations were determined using a standard curve from serial dilutions of chondroitin-4-sulfate (Millipore Sigma; Burlington, MA). Mean sGAG concentrations for each sample were calculated and used in statistical analysis.

c. Stimulation of Product without the Presence of Explants:

To determine if there was a response to reIL-1β from the umbilical-derived tissue allograft alone, without the presence of explant articular tissue, the following groups were cultured for 48 hours at 37 °C, 5% CO2, followed by sGAG determination as described above: 1) media+PBS, 2) media+PBS+reIL-1β 3) media+equicenta™CTM and 4) media+equicenta™CTM+reIL-1β. PBS or equicenta™CTM was added at a 1:5 ratio into DME media with 10% FCS, penicillin and streptomycin (100 U/ml). Recombinant equine IL-1β was added to the respective groups at a concentration of 10 ng/ml.

d. Histological Analysis:

Cartilage and synovial explants were placed in 10% formalin for 24 hours. Tissues were processed by the histology lab within the Louisiana State University Animal Diagnostic Laboratory. Cartilage tissue was decalcified prior to paraffin embedding using 15% formic acid solution in water in a 20:1 solution-to-tissue volume ratio at room temperature. The timing of decalcification was determined by frequent assessment of needle penetration; the needle passed smoothly through the sample without resistance. Cartilage and synovial explants were cut into 5 m sections and stained with toluidine blue and hematoxylin and eosin, respectively. Each slide was scanned using the Motic Easyview Imager (Motic, Inc; Emeryville, CA). Images were assessed and semi-quantitatively scored in a blinded manner by two investigators. Cartilage sections were evaluated for GAG intensity staining, chondrocyte morphology (pyknosis) and the presence hypocellularity (percentage of empty lacunae). GAG staining intensity was determined for each of the following cartilage zones: superficial, outer intermediate, inner intermediate, and deep. Scores, modified from a previous report evaluating osteoarthritic cartilage in horses, reflected the degree of metachromatic staining and included the following: 0=no staining (GAGs not present), 1=minimal staining (≥75% loss from normal cartilage staining), 2=mild staining (≥50% loss of from normal cartilage staining), 3=moderate staining (≥25% loss of from normal cartilage staining), and 4=marked staining (normal, strong GAG staining). The total GAG score was determined as the sum of all 4 cartilage zones. Hypocellularity of the cartilage was assessed as the percentage of empty lacunae, reflecting cellular loss or apoptosis. Scoring for hypercellularity at each zone was as follows: 0=0% cell reduction (normal, no empty lacunae), 1=1-25% cell reduction, 2=26-50% cell reduction, 3=51%-75% cell reduction and 4=76-100% cell reduction. Synovial sections were semi-quantitatively assessed for villus hyperplasia, characterized by increased layers of synoviocytes at the surface, and villus hypertrophy, evidenced by the presence of edema or clubbing/thickening of the villus overall. The presence of villus hyperplasia and hypertrophy was determined independently with scores including 0=not present, 1=minimally noted, 2=mildly present, 3=moderately present, and 4=markedly noted. Total synovial score was determined by calculating the sum of the hyperplasia and hypertrophy semi-quantitative scores.

e. Statistical Analysis:

The data were tested for normality using the Shapiro-Wilk test. PGE2 and sGAG data were analyzed using a nested ANOVA for horse, followed by Tukey's multiple comparisons to test for differences between groups. For analysis of sGAG concentration in Equicenta™ CTM or PBS only (without articular tissue), data were analyzed using a one-way ANOVA with Tukey's multiple comparisons test to evaluate differences across groups. A Mann-Whitney test was used to compare scores of GAG intensity and synovial histology between the PBS and equicenta™CTM groups, stimulated with or without reIL-1β. Significance was set at p<0.05 for all tests. Data were analyzed using GraphPad Prism (version 10.6; GraphPad Software, LLC; LaJolla, CA).

### 3. Results

a. PGE2 Concentrations in Supernatant:

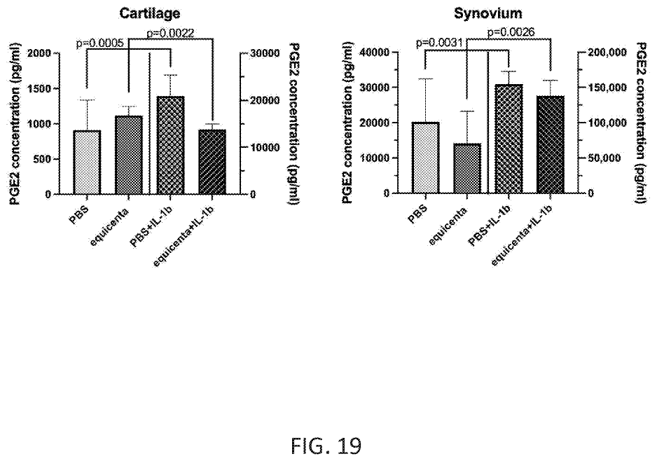

There was no effect on PGE2 concentrations in supernatant from cartilage explants exposed to equicenta™CTM (1117±127 pg/ml) compared to PBS (909±428 pg/ml; p=0.9997) or from synovial explants exposed to equicenta™CTM (14,143±9111 pg/ml) compared to PBS (20,263±12,199 pg/ml; p=0.8879) (FIG. 19). There was a 17.5 fold increase in PGE2 concentration from supernatant of cartilage explants stimulated with reIL-1β when compared to unstimulated explants (FIG. 2; p=<0.0005 for the PBS group and p=0.0022 for the equicenta™CTM group3); however, there was no significant effect (p=0.3083) on PGE2 concentrations from cartilage explant exposure to equicenta™CTM (13,763±1212 pg/ml) compared to PBS (20,892±4485 pg/ml). There was an 8.7 fold increase in PGE2 concentration from supernatant of synovial explants stimulated with reIL-1β (p<0.0031 for the PBS group and p=0.0026 for the equicenta™CTM group-8); however, there was no difference (p=0.8879) in PGE2 concentrations from synovial explants between exposure of equicenta™CTM (138,159±21,810 pg/ml) compared with PBS (154,405±18,281 pg/ml).

1. sGAG Concentrations in Supernatant:

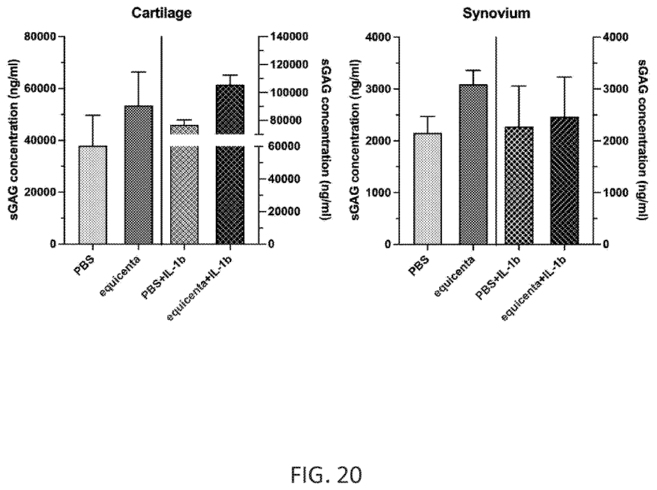

There was no difference in sGAG concentrations in supernatant from cartilage explants exposed to PBS or equicenta™CTM with or without reIL-1β stimulation. The mean concentration of sGAG in the supernatant from PBS-exposed cartilage explants was 38,058±11,684 ng/ml, compared with 53,509±12,914 ng/ml in equicenta™CTM exposed group; no significant difference was detected (p=0.9979). When stimulated with reIL-1β the mean concentration of sGAG was 76,861±6158 ng/ml for the PBS-exposed group compared with 105,695±6789 ng/ml for the equicenta™CTM exposed group (FIG. 20); no significant difference was detected (p=0.7494). Although not significantly different, the supernatant from cartilage explants had 2-fold the sGAG concentration after 48-hours of stimulation with reIL-1β compared to the unstimulated group. The concentration of sGAG from the synovial explants was lower than that from cartilage. There was no difference (p=0.6561) in sGAG concentrations in supernatant from synovial explants exposed to PBS (2152±556 ng/ml) or equicenta™CTM (3092±462 ng/ml; FIG. 3) and there was no effect (p>0.1) of reIL-1β stimulation on sGAG concentration in supernatant from synovial explants.

b. Stimulation of Product without Articular Explants:

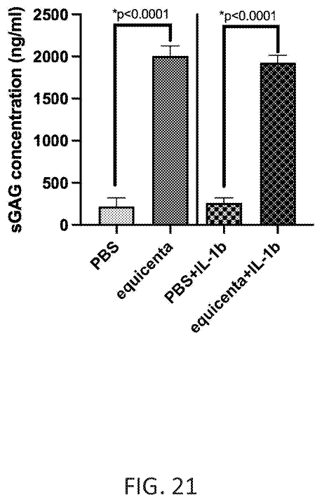

Concentrations of sGAG in equicenta™CTM in media (2008±119 ng/ml) was significantly increased 10-fold (p<0.0001) compared to PBS in media (219±103 ng/ml) (FIG. 21). Stimulation with reIL-1β did not significantly alter the concentration of sGAG in PBS+media (260±61 ng/ml; p=0.9894) or equicenta™CTM+media (1928±87; p=0.9323). The small amount of sGAG present in the PBS+media groups is likely attributable to FCS in the media used for the 1:5 dilution.

### 2. Histomorphologic Findings:

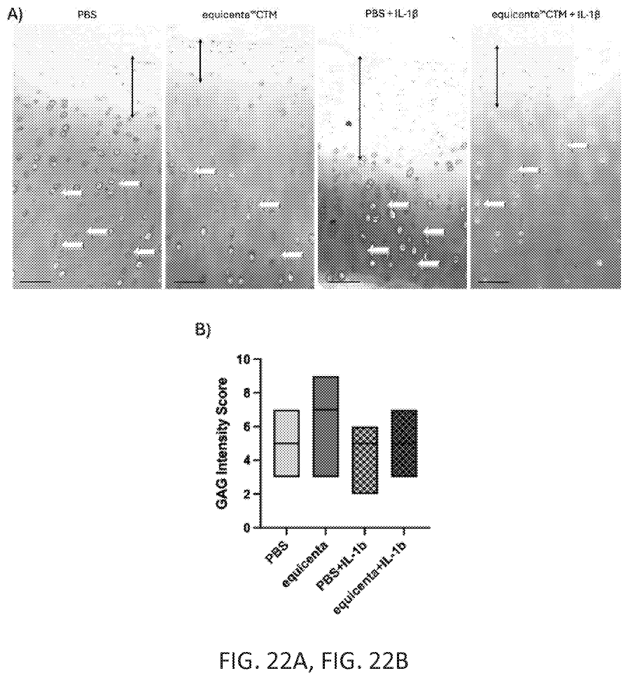

Total GAG intensity scores for cartilage explants did not differ significantly between groups (FIG. 22). There was no significant effect on total intensity GAG scores for cartilage explants stimulated with reIL-1β, as the median score for the PBS-exposed group was 5 (IQR: 3-5.5) and the median score for the equicenta™CTM exposed group was also 5 (IQR: 4-6.5). Empty lacunae were not noted in the cartilage sections from any of the groups and cellular morphology did not show crenation or intense dark stain indicating pyknosis. Subjectively, there were individual cartilage explants with greater GAG loss (less toluidine blue staining) noted throughout the outer intermediate and into the inner intermediate zone in the PBS group stimulated with reIL-1β compared to equicenta™CTM exposed group (FIG. 5). Total synovial scores did not differ (data not shown), with a median score of 0 across all groups evaluated. Synovial hypertrophy was not observed in any sections. Only minimal synovial hyperplasia was noted in a few selected areas across three explants from different treatment groups.

### 4. Discussion

This study evaluated a cryopreserved umbilical-derived matrix product, equicenta™CTM, in an ex vivo explant/IL-1β model of arthritis to determine its effect on equine cartilage and synovium. A recent study reported that equicenta™CTM contains several important extracellular matrix and anti-inflammatory proteins at moderate to high abundance, including chondroitin sulfate proteoglycan, various collagens, small leucine-rich proteoglycans, such as biglycan, decorin, and lumican, alpha-2-macroglobulin (A2M), and annexin Al. Therefore, it was expected that equicenta™CTM may alter the pro-inflammatory and catabolic responses of IL-1β on equine cartilage and synovial explants.

Synovial fluid from osteoarthritic joints has been reported to have significant increases in PGE2 compared to healthy joints of horse, suggesting that PGE2 would be a valid marker of osteoarthritis and articular inflammation; therefore, to assess the inflammatory response, PGE2 concentrations in the supernatant from explants exposed to equicenta™CTM with and without IL-13 were determined. Equicenta™CTM exposure did not increase PGE2 concentrations in the supernatant from cartilage or synovial explants, indicating that administration of the umbilical-derived tissue allograft did not elicit a prostaglandin-associated inflammatory response. This finding is consistent with previous in vivo studies, in which intra-articular injection of equicenta™CTM in clinically normal and a limited number of diseased joints did not increase the pro-inflammatory cytokines IL-2, IL-8, and IL-15 after injection. Interestingly, in that study, there were significant increases in interleukin-1 receptor antagonist (IL-1Ra) in the synovial fluid after equicenta™CTM injection on days 1, 3, and 10 compared with baseline measurements and with findings from the saline control group. IL-1Ra concentrations were not determined in this ex vivo study, but if an increase in IL-1Ra was present, it was not sufficient to inhibit the effects of IL-1β on PGE2 production, as no differences in concentration of PGE2 from cartilage or synovial explants were noted between groups. Unexpectedly, other anti-inflammatory mediators, such as A2M or annexin A1, present in equicenta™CTM also did not appear to affect IL-1β stimulation of PGE2 production. Therefore, additional studies evaluating varying doses of IL-1β and/or other arthritis models may further elucidate whether there is potential value of these anti-inflammatory proteins in equine CTM. Importantly, equicenta™CTM did not result in an increase in the inflammatory mediator PGE2- and did not exacerbate the inflammatory effect of reIL-1β from articular tissue in this study.

Increased PGE2 concentrations after stimulation with reIL-1β supported that the inflammatory insult to induce a functional ex vivo model of articular inflammation/arthritis was applied to cartilage and synovial explants in this study. Inflammation was induced with the addition of IL-1β as evidenced by a significant 17-fold increase in PGE2 concentrations from cartilage explants and an 8-fold increase from synovial explants after 48 hours of exposure. While not measured, IL-1β stimulation would have also been expected to increase production of other inflammatory mediators, including IL-6, IL-8, and tumor necrosis factor-α in cartilage, as their increases have been reported to occur within 24 hours of stimulation. Stimulation with reIL-1β for 48 hours also resulted in a 2-fold increase in soluble sGAG concentrations in the supernatant. Increased sGAG concentrations from cartilage supernatant after 48 hours of stimulation with reIL-1β could result in a catabolic effect similar to reports that demonstrated increased soluble sGAG within the supernatant of reIL-1β-stimulated cartilage. Although there was no significant decrease in the visual GAG intensity scores from cartilage explants exposed to reIL-1β, the lowest score was noted in a section from the PBS-exposed, reIL-1β-stimulated group. Longer incubation times may be needed to visually detect the loss of GAG staining in articular cartilage due to the large amount of residual GAG, particularly in the deeper zones. Additionally, the release of sGAG from the product likely contributed to the overall sGAG concentration in the supernatant of cartilage explants exposed to equicenta™CTM, potentially reducing the contribution of sGAG from cartilage. Furthermore, semi-quantitative subjective grading of histological sections may be insufficiently sensitive to detect early GAG loss or its suppression.

Although not significantly increased, higher concentrations of sGAG were noted in the supernatants from cartilage explants exposed to equicenta™CTM. To better determine the source of the sGAG an additional experiment was conducted without the presence of explant tissues. Results demonstrated that equicenta™CTM incubated in media at 37 °C for 48 hours yielded a significant 10-fold increase in sGAGs compared to PBS in media. It is suspected that sGAGs released from the umbilical tissue allograft itself may have contributed to a portion of the total sGAG concentration amounts from the supernatant of cartilage explants. Polysulfated glycosaminoglycans (sGAGs), a therapy for osteoarthritis in horses, have been reported to upregulate sGAG production and may serve as a mechanism for increased sGAG production in vivo. Additional studies, including the radiolabeled sulfate incorporation assay, would be necessary to further elucidate potential mechanisms for the sGAG increase, but are beyond the scope of this study.

Study limitations include a small horse sample size, evaluation of the product at the single concentration that had safety data for clinical use, use of pre-exposure and assessment of single, although sentinel, biomarkers of inflammation and cartilage health. Similar to other studies, to increase sample size, multiple explants were obtained from three horses, yielding a final sample size of nine per outcome. Explants were evaluated using a single concentration of equicenta™CTM that had safety data for clinical use. PBS and equicenta™CTM was applied at a 1:5 dilution of product to culture media as an estimate of the concentration that may be present in a clinically treated joint, corresponding to a dose of 1.5 into 7.5 mL of synovial fluid. Higher concentrations of the product may have produced different results and remain to be studied. Explants in this study were also pre-exposed to PBS or equicenta™CTM for 48 hours prior to stimulation with reIL-1β, which allowed the evaluation of the product for its potential use as a protective therapeutic and to permit time for any tissue interactions to occur. It remains to be determined if application of equicenta™CTM at a time when inflammation is already established would produce different results. PGE2 was the pro-inflammatory mediator assessed in this study to evaluate the effects of equicenta™CTM on cartilage and synovial tissue in an ex vivo model of arthritis and evaluated the prostaglandin pathway of inflammation.

### 5. Conclusions:

Equicenta™CTM did not increase PGE2 concentrations in the supernatant from equine cartilage or synovium, indicating lack of stimulation of the inflammatory prostaglandin response from these tissues directly exposed to clinically-relevant amounts of umbilical-derived tissue allograft. These findings supported previous work demonstrating equicenta™CTM's biocompatibility in equine joints in vivo. In addition to the numerous previously reported extracellular matrix proteins in equicenta™CTM, this study demonstrated the natural release of sGAGs from the product over 48 hours at 37 °C. Polysulfated sGAG can provide protective benefits in horses with joint injury or osteoarthritis.

Those skilled in the art will recognize, or be able to ascertain using no more than routine experimentation, many equivalents to the specific embodiments of the method and compositions described herein. Such equivalents are intended to be encompassed by the following claims.

1. A composition comprising a Fillet-Derived Umbilical Tissue (FDUT).

2. The composition of claim 1, wherein the FDUT is derived from an umbilical cord.

3. The composition of claim 1, wherein the umbilical cord is a mammalian umbilical cord.

4. The composition of claim 3, wherein the mammal is selected from the group consisting of human, equine, bovine, porcine, ovine, caprine, canine, and feline.

5. The composition of claim 1, wherein the FDUT comprises a connective tissue portion.

6. The composition of claim 5, wherein the connective tissue portion comprises an outer layer of intervascular and an inner layer of perivascular connective tissue.

7. The composition of claim 1, wherein the FDUT is native to a fillet portion of an umbilical cord and has had the amnion and umbilical vessels removed.

8. The composition of claim 1, wherein the intervascular and perivascular connective tissue are retained following removal of the amnion and vessels.

9. The composition of claim 1, wherein the composition does not comprise tissue from a branch portion of an umbilical cord.

10. The composition of claim 1, wherein the FDUT is partially devascularized and is substantially free of an external amniotic membrane, containing about 0% to 2% by wet weight of residual amnion based on the starting tissue material.

11. The composition of claim 1, wherein the FDUT is substantially free of epithelial and endothelial cells.

12. (canceled)

13. The composition of claim 1, wherein the FDUT provides a structural matrix and/or suspension for tissue support and cell scaffolding migration, said suspension and/or matrix comprising cells and a profile of native growth factors and/or exosomes that are released as the matrix biodegrades in-vivo.

14. The composition of claim 1, wherein the FDUT comprises functional proteins, growth factors, and/or exosomes native to umbilical connective tissue.

15. The composition of claim 13, wherein the profile of functional proteins comprises one or more of metalloproteinases-2 (TIMP-2), tissue inhibitor of metalloproteinases-1 (TIMP-1), and alpha-2-macroglobulin (A2M).

16. (canceled)

17. The composition of claim 13, wherein the profile of native growth factors comprises a pro-regenerative payload including IGF-1, bFGF, and TGF-β1.

18. (canceled)

19. (canceled)

20. (canceled)

21. The composition of claim 1, wherein the FDUT is preserved by one or more methods selected from the group consisting of cryopreservation, lyophilization, vitrification, supercritical drying, and desiccation.

22-24. (canceled)

25. The composition of claim 1, further comprising a cryopreservation solution, wherein the cryopreservation solution optionally comprises non-cell permeating cryopreservatives.

26. (canceled)

27. (canceled)

28. A method of treating a condition in a mammal, the method comprising administering an effective amount of the composition of claim 1 to a target site in the mammal.

29-49. (canceled)

50. A method of treating an equine musculoskeletal condition comprising administering an effective amount of the composition of claim 1 to a target site selected from an articular joint, tendon, or ligament.

51. (canceled)

52. A method of rejuvenating and restoring atrophied or poor quality healed tissue defects comprising administering the composition of claim 1 to a subject in need thereof.

53-82. (canceled)
